(19) United States
(12) Patent Application Publication
(10) Pub. No.: US 2022/0364125 A1
(43) Pub. Date: Nov. 17, 2022

(54) GENOME ENGINEERING THE HUMAN IMMUNOGLOBULIN LOCUS TO EXPRESS RECOMBINANT BINDING DOMAIN MOLECULES

(71) Applicant: University of Southern California, Los Angeles, CA (US)

(72) Inventors: Paula Cannon, Los Angeles, CA (US); Geoffrey Rogers, Los Angeles, CA (US)

(21) Appl. No.: 17/815,711

(22) Filed: Jul. 28, 2022

Related U.S. Application Data
(63) Continuation-in-part of application No. PCT/US2021/015489, filed on Jan. 28, 2021.
(60) Provisional application No. 62/967,018, filed on Jan. 28, 2020.

(51) Int. Cl. C12N 15/90; C12N 15/10; C07K 16/10; A61K 35/17

(52) U.S. Cl. CPC C12N 15/907; C12N 15/102; C07K 16/1045; A61K 35/17; C12N 2310/20; C12N 2800/80; C07K 2317/622; C07K 2317/569; C07K 2317/76

(57) ABSTRACT

The disclosure describes a genome engineering strategy that allows for the production of secreted antibody fragments or non-immunoglobulin binding domains and the corresponding cell surface B cell receptor (BCR) from a human immunoglobulin (Ig) locus, and uses thereof.

### CROSS REFERENCE TO RELATED APPLICATIONS

This application is a continuation-in-part of International Application No. PCT/US2021/015489, filed Jan. 28, 2021, which application claims priority under 35 U.S.C. § 119 from Provisional Application Ser. No. 62/967,018, filed Jan. 28, 2020, the disclosures of which are incorporated herein by reference.

### STATEMENT OF GOVERNMENT SUPPORT

This invention was made with Government support under RO1 DE025167, awarded by the National Institutes of Health/National Institute of Dental and Craniofacial Research. The Government has certain rights in the invention.

### TECHNICAL FIELD

The disclosure describes a genome engineering strategy that allows for the production of secreted antibody fragments (e.g., single chain antibodies) and binding domains including non-immunoglobulin binding domains, as well as corresponding cell surface B cell receptor (BCR) from a human immunoglobulin (Ig) locus, and uses thereof.

### INCORPORATION BY REFERENCE OF SEQUENCE LISTING

Accompanying this filing is a Sequence Listing entitled, “00130-034US1.xml” created on Jan. 27, 2022 and having 56,136 bytes of data, machine formatted on IBM-PC, MS-Windows operating system using WIPO Standard ST.26 formatting. The sequence listing is hereby incorporated by reference in its entirety for all purposes.

### BACKGROUND

B cells naturally generate a vast repertoire of antibodies with different specificities through a complex process involving recombination and mutagenesis of common starting sequences in the immunoglobulin (Ig) locus. A specific antibody variant is displayed on the surface of a B cell in the form of a B cell receptor (BCR), and engagement of the BCR with a corresponding antigen leads to activation of that cell and the secretion of its antibody. The antibody repertoire in the body is available for the selection of extremely specific responses to, for example, infectious diseases. Moreover, B cells' responses also evolve over time, and generate antibody secreting descendants that are capable of surviving and producing antibodies for decades, as well as memory responses that can be recalled upon antigen re-encounter.

In addition, specific “pre-formed” antibodies with desirable properties can be used as therapies when injected as recombinant protein drugs. These antibody drugs are used for example to treat cancer, infectious diseases and autoimmune diseases. This approach provides both passive immunization, as well as allowing the use of antibodies with unique properties that do not efficiently form in nature. A good example of the latter case are so-called ‘broadly neutralizing’ antibodies (bnAbs) directed against HIV. bnAbs are rare antibodies that can inhibit many different strains of HIV but do not form easily during natural infections. In addition to delivery as recombinant proteins, antibody therapies are also being developed using gene therapy approaches, where the desired antibody is secreted from cells in the body such as muscle cells.

### SUMMARY

The disclosure provides compositions and method for genome engineering to edit the human immunoglobulin (Ig) locus, and thereby allow the expression of antibodies (e.g., therapeutic antibodies, antibody fragments and non-immunoglobulin binding domains) from the natural antibody locus, the Ig locus. In a particular embodiment, the disclosure provides for the use of single chain and/or single domain antibodies (sdAbs) and which provide the antibody functionality. The disclosure provides a genome engineering strategy that allows for the insertion of recombinant polynucleotide cassettes to create antibody, antibody fragments, antibody-like molecules and non-immunoglobulin binding domains within the Ig locus such that secreted antibody or antibody fragments are produced as well as the production of corresponding cell surface B cell receptor (BCR). Having both of these alternatively spliced forms of the sdAb is important since it means that an engineered B cell will be able to respond to the presence of the matched antigen, which in turn means that the B cell could be amplified in vivo, secrete the recombinant antibody or fragment thereof, develop memory, and be subject to ongoing affinity maturation to alter/improve the specificity of the antibody. Also provided herein, are studies demonstrating the effectiveness of a therapeutically relevant recombinant antibody or fragment thereof disclosed herein which recognized the Env protein from the human immunodeficiency virus (HIV). The methods and compositions of the disclosure provided for the expression of a functional anti-HIV recombinant antibody or fragment thereof from the engineered cells that inhibited HIV replication. Accordingly, by using a recombinant antibody or fragment thereof and the immunoglobulin editing approaches disclosed herein, a major technical challenge inherent in editing the Ig locus was overcome. Further, the immunoglobulin editing approaches of the disclosure are broadly applicable, and can be used as a platform technology for genome editing of B cells and their precursor cells to express specific antibodies (e.g., therapeutic antibodies) and antibody-like constructs.

In a particular embodiment, the disclosure provides a method for the production of recombinant antibodies or fragments thereof from an immunoglobulin locus, comprising: introducing a targeted DNA break in a constant region downstream of the CH1 exon of an immunoglobulin locus using a genome editing system; and inserting a promoter-driven expression construct that expresses an antigen-binding domain (e.g., a VHH domain) into the genome edited immunoglobulin locus, wherein the promoter-driven expression construct produces an mRNA that lacks the CH1 exon but comprises the Hinge, CH2, CH3 exons of the immunoglobulin locus. In a further embodiment, the immunoglobulin locus is a human immunoglobulin locus. In yet a further embodiment, the immunoglobulin locus is selected from the IGHG1, IGHG2, IGHG3, IGHG4, IGHD, IGHE, IGHM, IGHA1, and IGHA2. In another embodiment, the immunoglobulin locus is selected from the IGHG1, IGHG2, IGHG3, and IGHG4. In a certain embodiment, the immunoglobulin locus is IGHG1. In another embodiment, the genome editing system is selected from CRISPR/Cas9, CRISPR/Cpf1, Zinc finger nucleases, and transcription activator-like effector nucleases (TALEN). In yet another embodiment, the genome editing system is a S. pyogenes (sp) CRISPR/Cas9 genome editing system. In a certain embodiment, the spCas9 guide RNAs have the sequence of sg01, sg02, sg03, sg04, sg05, sg06, sg12, sg16, or sg17 presented in Table 2. In another embodiment, the genome editing system is a CRISPR/Cpf1 genome editing system. In a certain embodiment, the Cpf1 guide RNAs have the sequence of Cpf1-g1, Cpf1-g2, Cpf1-g3, or Cpf1-g4 presented in Table 3. In yet another embodiment, the gRNAs have the sequence of IgG4end-g1 to g5 (see Table 6). In yet another embodiment, the targeted DNA break in a constant region downstream of the CH1 exon is between the CH1 exon and Hinge exon of the immunoglobulin locus. In yet another embodiment, the targeted DNA break in a constant region downstream of the Hinge exon is between the Hinge exon and CH2 exon of the immunoglobulin locus. In yet another embodiment, the targeted DNA break is between the CH2 and CH3 exon of the immunoglobulin locus. In still another embodiment, the targeted break is in the intron between CH2 and CH3. The approach of targeting the intron between CH2 and CH3 has an advantage that it allows insertion of more than just an antigen-binding domain; specifically, the inserted sequence also can comprise the Hinge and CH2 domains of the antibody. This allows for the customization of additional parts of the constant region of the antibody. This can be useful to introduce mutations into the CH2 that enhance properties of the antibody such as ADCC. In still another embodiment, the targeted DNA break is downstream of the CH3 exon. FIG. 19 shows, for example, locations for DNA breaks and insertions of an antigen recognition cassette. In a certain embodiment, the promoter-driven expression construct is inserted into the immunoglobulin locus by homology-directed repair of sequence-specific DNA breaks generated by ZFNs, TALENs, or CRISPR/Cas. In another embodiment, the promoter-driven expression cassette is inserted into the immunoglobulin locus at the site of the sequence-specific DNA break by NHEJ-mediated ligation and end capture. In another embodiment, the promoter-driven expression construct comprises a B cell specific promoter. Examples of B cell specific promoters include, but are not limited to, EEK and MH. In yet another embodiment, a cell comprising the promoter-driven expression construct produces an mRNA that optionally further comprises the M1 and M2 exons of an immunoglobulin locus.

In another embodiment, the disclosure provides methods and composition for generating/editing IgG4. In one embodiment, a cassette comprising a binding domain is inserted downstream of CH2 or CH3 domains of IgG4. In a further embodiment, the gRNA sequences used in editing the IgG4 comprise the sequences in Table 6.

In a particular embodiment, the disclosure also provides a method to produce an engineered B cell or an engineered precursor B cell that expresses an antibody or fragment thereof, comprising: treating a B cell or a precursor B cell using a genome editing method described herein. In a further embodiment, the B cell or the precursor B cell is treated in vitro. In another embodiment, the precursor B cell is a hematopoietic stem cell or induced stem cell.

In a certain embodiment, the disclosure provides for an engineered B cell or an engineered precursor B cell that expresses a recombinant antibody or fragment thereof made by a method described herein.

In a particular embodiment, the disclosure also provides for a cell line comprising an engineered B cell or an engineered precursor B cell described herein. The engineered precursor B cell can be an embryonic stem cell, an induced pluripotent stem cell, or a hematopoietic stem cell. Methods of isolating embryonic stem cells are known in the art. Methods of generating induced pluripotent stem cells are known in the art (see, e.g., U.S. Pat. Nos. 9,862,930; 9,005,966; the disclosure of which are incorporated herein by reference).

In a certain embodiment, the disclosure provides for antibodies or fragments thereof isolated from an engineered B cell, from an engineered precursor B cell, or from a cell line disclosed herein.

In another embodiment, the disclosure further provides a method of treating a subject with a microbial or viral infection, comprising: isolating B cells or precursor B cells from the subject; treating the isolated B cells or precursor B cells from the subject using a method disclosed herein to produce engineered B cells that express an antibody or fragment thereof that recognize antigen(s) from an infectious microbe; administering the engineered B cells to the subject. In a further embodiment, the subject has a viral or bacterial infection. In yet a further embodiment, the viral infection is HIV, Hepatitis, Herpes simplex, Ebola, Dengue, influenza, and coronavirus. In another embodiment the disclosure provides methods of treating a second subject with a microbial or viral infection, comprising: isolating B cells or precursor B cells from a first subject; treating the isolated B cells or precursor B cells from the first subject using a method disclosed herein to produce engineered B cells that express an antibody or fragment thereof that recognize antigen(s) from an infectious microbe; administering the engineered B cells to the second subject. In a further embodiment, the second subject has a viral or bacterial infection. In yet a further embodiment, the viral infection is HIV, Hepatitis, Herpes simplex, Ebola, Dengue, influenza, and coronavirus. In still another embodiment, the disclosure provides a method of engineering B cells and precursor B cells in vivo comprising administering a vector system comprising a vector containing an antigen recognition cassette of the disclosure in combination with genome editing components to enable site-specific insertion of the cassette, for example by homology directed DNA repair at a sequence-specific DNA break created by, e.g., CRISPR/Cas, Talen, ZFN etc. In another embodiment, the promoter-driven expression cassette is inserted into the immunoglobulin locus at the site of the sequence-specific DNA break by NHEJ-mediated ligation and end capture.

In another embodiment, the disclosure also provides a method of treating a subject with cancer, comprising: isolating B cells or precursor B cells from the subject; treating the isolated B cells or precursor B cells from the subject using a method disclosed herein to produce engineered B cells that expresses an antibody or fragment thereof that recognize antigen(s) from the cancer cells; administering the engineered B cells to the subject. In yet another embodiment, the subject has a cancer selected from non-Hodgkin's lymphoma, acute lymphoblastic leukemia, B-cell lymphoma, mantle cell lymphoma, multiple myeloma, acute myeloid leukemia, colorectal cancer, breast cancer, lung cancer, ovarian cancer, and renal cancer.

In a particular embodiment, the disclosure also provides a method of treating a subject with an autoimmune disorder, comprising: isolating B cells or precursor B cells from the subject; treating the isolated B cells or precursor B cells from the subject using a method disclosed herein to produce engineered B cells that expresses an antibody or fragment thereof that can bind to and prevent activation of cytokines or receptors associated with an autoimmune disorder; or prevent aggregations or plaques associated with an autoimmune disorder; administering the engineered B cells to the subject. In yet another embodiment, the subject has autoimmune disorders selected from Alzheimer's disease, Celiac disease, Addison disease, Graves disease, dermatomyositis, multiple sclerosis, rheumatoid arthritis, psoriasis, and inflammatory bowel disease.

In another embodiment, the disclosure provides methods and compositions to generate CrossMab constructs that can be expressed from a recombinant cell. In one embodiment, the method comprises inserting a cassette comprising binding domains downstream of the CH1 exon.

### DESCRIPTION OF DRAWINGS

FIG. 1 presents the capabilities of B cells to be reprogrammed using genome engineering. Naïve B cells reprogrammed to express a pre-designed BCR through genome editing, for example an anti-HIV bnAb, could recapitulate the normal functions of a B cell. These include: (1) the generation of immunological memory—memory B cells generated through a primary vaccination can persist for prolonged periods in the body, remaining primed to respond to antigen re-exposure; (2) plasma cell differentiation—plasma cells that differentiate from a germinal center reaction can survive for decades in the body, continually secreting antibodies into the blood that provide constant surveillance and effector function against their target, whether that is a viral antigen or other molecule (cancer, inflammatory molecule, etc.) which is detrimental to the body; (3) affinity maturation—during a germinal center reaction after vaccination, engineered B cells could undergo somatic hypermutation, and through selective processes novel antibody sequences could emerge with greater specificity than the input antibody engineered into the cells. This process could be particularly valuable in the case of HIV or other hypermutagenic viral infections, or a cancer where the target antigen could mutate, as it may allow engineered B cells to continue to evolve their specificity to prevent mutations from escaping recognition by the engineered antibody.

FIG. 2A-D provides a comparison of conventional antibodies and examples of single chain antibodies and antibody-like molecules. (A) Conventional human antibodies comprise two light (L) and two heavy (H) chains, and their antigen-binding specificity is conferred by the combination of the variable (V) regions in both the light and heavy chains. The light chain consists of an antigen-binding variable domain (VL) and a constant domain (CL); both domains interact with the heavy chain. Similarly, the heavy chain has an antigen-binding variable domain (VH), but several constant region domains (i.e., CH1, Hinge, CH2, and CH3 in the case of IgG1, as shown). (B) In contrast, single chain antibodies, for example single-domain antibodies (sdAbs) originating in camelids consist of only a heavy chain, with an antigen-binding VHH domain and a constant region comprising Hinge, CH2 and CH3 domains, but which lacks the CH1 domain that is used for light chain pairing in the conventional H plus L antibodies. Similar molecules can also be generated using alternate antigen recognition domains such as single chain variable fragments (scFvs) in place of the VHH domain. (C) Antibody-like molecules can also be created in a single-chain format, by linking H chain components (e.g., Hinge, CH2, CH3) with a non-antibody derived protein domain such as a soluble receptor derivative, a therapeutic protein, or other protein domain to generate Fc-fusion proteins. (D) Single-chain antibodies are also amenable to tandem multiplexing, using flexible amino acid linkers to connect multiple functional domains. Illustrated here are: a bispecific antibody with 2 different VHH domains (VHH-1 and VHH-2), a bi-valent antibody with 2 tandem copies of the same VHH domain, and a hybrid tandem construct containing both a VHH domain and a receptor domain. Other combinations are also feasible, combining for example up to 4 tandem VHH domains.

FIG. 3 demonstrates HIV-specific broadly-neutralizing single domain antibodies (bn-sdAbs) that neutralize two different strains of HIV. The sdAbs comprised VHH domains which were recreated from the published protein sequences (J3, A14, B9, 3E3; McCoy et al. 2014 PLoS Pathog 10:e1004552) or nucleotide sequences (9, 28, A6; Koch et al. 2017 Sci Rep 7:8390) and the sequence for the Hinge-CH2-CH3 domains of human IgG1, then produced by transfection of 293T cells with plasmids containing expression cassettes for the indicated sdAbs and the resulting supernatants tested for anti-HIV activity using a GHOST cell assay (Cecilia et al. 1998 J Virol 72:6988-6996). All sdAbs were inhibitory against both strains of HIV tested, though some were more effective against JR-CSF (9, 28, A6) whereas other were more effective against NL4-3 (J3, A14, B9, and 3E3). The ACH1 supernatant is a negative control generated from cells transfected with plasmids expressing just the Hinge-CH2-CH3 domains of human IgG1 and lacking an anti-HIV binding domain. eCD4-Ig (eCD4) was included as a positive control secreted protein known to neutralize many strains of HIV.

FIG. 4A-B provides a schematic of genome editing at the IGHG1 locus within the intron preceding the hinge exon to create a single chain antibody. As an example, the use of a VHH domain is shown to create an sdAb, although other antibody or protein domains, including those described in FIG. 2, could be used in place of the VHH domain. (A) antibody fragments (e.g., sdAbs) can be created at the human Ig locus using genome engineering based, for example, on homology-directed repair (HDR) catalyzed by site-specific DNA double-stranded breaks produced by a targeted nuclease such as CRISPR/Cas9. The recombinant, e.g., sdAb VHH, cassette is provided using a homology donor template, which consists of a promoter (in these examples a B cell-specific EEK promoter), a functional domain (for example a VHH domain) and a splice donor, and is flanked by sequences with homology to the Ig locus (homology arms). Following HDR, the VHH cassette is inserted between the CH1 and Hinge exons of IgG1 in the human genome, as indicated. (B) After site-specific insertion of the cassette into the genome, the inserted promoter drives transcription, and the splice donor after the VHH exon splices the resulting RNA transcript with the downstream Hinge, CH2, and CH3 exons to produce an antibody fragment (e.g., sdAb-IgG1 antibody). Exclusion of the membrane exons M1 and M2 results in production of the secreted form of the antibody fragment, while their inclusion results instead in the transmembrane BCR.

FIG. 5 demonstrates the activity of spCas9 complexed with guide RNAs (gRNAs) at on- and off-target IgG genes. The activity of 10 spCas9 gRNAs (described in Table 2) targeting the desired intron of IgG1 were assessed in K562 cells at the on-target IGHG1 gene site, as well as at 4 major predicted off-target regions (IGHG2, IGHG3, IGHG4, and IGHGP) by Sanger sequencing (Hsiau et al., bioRxiV, 2019, DOI:10.1101/241082). On-target DSBs were generated by all guides, though the total detectable activity varied. Three guides (sg02, sg03, and sgCOR2) exhibited off-target activity at one or more of the homologous IgG genes, whereas the other 7 showed little to no off-target cutting as detected by this assay (limit of detection ˜2%).

FIG. 6A-C provides a schematic of examples of homology donor designs. To survey the ability of specific gRNAs to support site-specific genome editing subsequent to DSB formation, a series of matched homology donor plasmids can be created. These can contain, for example, GFP expression cassettes driven by the ubiquitous PGK promoter, to allow quantification of successful genome editing rates by flow cytometry for GFP expression. The GFP cassettes are flanked by homology arms, e.g., DNA sequences that match the DNA sequence flanking the selected gRNA target sequence and DNA break site. Homology arms can vary in size, and optimal lengths of the arms can be determined by comparing the function of different designs of homology donors. (A) spCas9 produces a DNA DSB between bp −3 and −4 from the PAM sequence, as illustrated with a sample gRNA target sequence (SEQ ID NO:1). (B) Location of a sample gRNA target sequence between the CH1 and Hinge (H) exons of IGHG1. (C) A series of four different homology donor designs are shown, with different left and right arm lengths (i.e., 500/500, 750/750, 1000/500, and 500/1000). The position of the PGK-GFP cassette insertion, between bp −3 and −4 of the gRNA target sequence is indicated in the first example (SEQ ID NO:2). Separating the gRNA target sequence and PAM on either side of the GFP cassette means that Cas9 will be unable to cut the homology donor, or the genomic DNA following successful homology-directed repair genome editing. A separate series of homology donors with the different arm lengths were generated for each gRNA to be tested, since the exact DNA break site varies, and thus the location of the GFP gene insertion cassette within the homologous sequence is also different for each gRNA.

FIG. 7 demonstrates genome editing at the IGHG1 locus in K562 cells using spCas9 complexed with gRNAs and matched plasmid homology donors. The gRNAs are described in Table 2. After 3 weeks, stable GFP expression, indicating site-specific genome editing, was measured by flow cytometry. The gRNA used was the most important source of variation in the final GFP levels; all homology arm designs for sg05 were superior to other gRNA/homology donor pairs. Data is from n=3 experiments.

FIG. 8A-D demonstrates genome editing at the IGHG1 locus using spCas9/gRNAs and adenovirus associated virus serotype 6 (AAV6) homology donors. (A) Provides a schematic of the AAV6 vector genomes in which homology donor cassettes were packaged into the vectors. (B) Stable GFP expression in K562 cells that were treated with spCas9 complexed with the indicated gRNAs and also transduced with matched AAV6 homology donor vectors and were measured after 3 weeks by flow cytometry. (C) The expected outcome of genome editing using these homology donors is shown, including a schematic of the design of the ‘in-out PCR’ assay used to confirm site-specific gene insertion. (D) In-out PCR demonstrates site-specific gene insertion in cells that received AAV6 homology donors and spCas9/gRNAs, but not in cells receiving AAV6 only, confirming that the PCR is amplifying DNA that is a result of site-specific gene insertion. Amplicons were resolved by agarose gel electrophoresis and are of the expected lengths for each gRNA (sg01: 1027 bp; sg04: 923 bp; sg05: 922 bp; sg12: 1171 bp; sg16: 931 bp).

FIG. 9 demonstrates that gene editing at the IGHG1 locus with spCas9/gRNAs and AAV6 homology donors is site-specific and precise. In-out PCR amplicons from FIG. 8 were subjected to Sanger sequencing in order to confirm that the expected site-specific insertions had occurred. Alignment of sequences to genomic DNA showed the PGK-GFP insert cassette precisely at the predicted spCas9 cleavage site for each gRNA (SEQ ID Nos: 3-7), as expected given the design of the homology donor constructs. Additionally, the clean traces indicate that the amplicon represents a homogeneous population of DNA products.

FIG. 10A-C demonstrates that genome editing at the IGHG1 locus produces HIV-specific bn-sdAbs in Raji cells. (A) Raji cells (a human B cell line) were nucleofected with RNPs comprising spCas9 and gRNA sg05 and the indicated plasmid homology donors, comprising either a PGK-GFP cassette (control) or the A6 or J3 VHH cassettes downstream of an EEK promoter. (B) A 10-fold increase in stable GFP expression after 2 weeks was observed in Raji cells receiving homology donor plasmids containing GFP expression cassettes plus sg05 RNPs compared to donor plasmid only, consistent with site-specific gene insertion stimulated by the targeted double-stranded DNA break (DSB). (C) Cells receiving Cas9 RNPs plus homology donor plasmids containing A6 or J3 VHH cassettes exhibited double-positive staining (gated) for both membrane-bound IgG expression and binding to soluble His-tagged HIV gp120.

FIG. 11A-B demonstrates genome editing at the IGHG1 locus produces HIV-specific bn-sdAbs in Ramos cells. (A) Increased stable GFP expression after 2 weeks was observed in Ramos cells (a human B cell line) receiving the GFP plasmid donor and Cas9 RNPs, consistent with site-specific gene insertion. (B) Ramos cells receiving Cas9 RNPs plus donor plasmids containing A6 or J3 VHH cassettes exhibited double-positive staining for both IgG expression and HIV gp120 binding.

FIG. 12A-C provides confirmation of site-specific genome editing in Raji cells. Raji cells from FIG. 10 were assayed at the DNA level for confirmation of site-specific genome editing. (A) Schematic of the anticipated genomic outcome following HDR for all 3 homology donors and including the location of primers for diagnostic in-out PCRs. As before, this PCR strategy will generate an amplicon after site-specific insertion of the GFP or VHH (A6 or J3) expression cassettes. (B) In-out PCR of gene edited Raji cells showed amplicons of the expected size for all 3 homology donors. (C) In-out PCR amplicons were subjected to Sanger sequencing to confirm that site-specific insertions had occurred. Alignment of sequences to genomic DNA showed insertion of the GFP or VHH cassettes precisely at the predicted spCas9 cleavage site, as expected given the design of the homology donor constructs (SEQ ID NO:8-9). Additionally, the clean traces indicate that the amplicon represents a homogeneous population of DNA products.

FIG. 13A-B demonstrates after enrichment, bn-sdAb (A6 or J3) but not GFP edited cells secrete human IgG. (A) The frequency of HIV-specific cells was measured by flow cytometry, based on ability to bind HIV gp120 protein, for Ramos and Raji cells. (B) Secreted antibodies were detected from both cell lines following engineering with the A6 or J3 VHH cassettes, but not from GFP-edited cells, consistent with these antibodies being produced as a consequence of site-specific genome editing as illustrated in FIG. 4.

FIG. 14A-B shows the anti-HIV activity of antibodies produced by engineered B cell lines (Ramos and Raji). (A) Effect of supernatants on HIV infection in GHOST cell assay for A6-containing supernatants. As a control, 293T cells were transfected with a plasmid expression cassette for the A6 sdAb. (B) Effect of supernatants on HIV infection for J3-containing supernatants. As a control, 293T cells were transfected with a plasmid expression cassette for the J3 sdAb.

FIG. 15 provides for the quantification of anti-HIV activity of bn-sdAbs produced by transfection of 293T cells and genome editing of Raji and Ramos cells. The relative efficiency of each antibody against the indicated strain of HIV was conserved regardless of whether it was produced in 293T cells by transfection or from genome edited B cells.

FIG. 16A-D demonstrates genome editing and in vitro differentiation of primary human B cells. (A) Timeline of B cell activation and gene editing. (B) Stable GFP expression in primary human B cells after genome editing with CCR5-specific ZFN mRNA and matched AAV6-CCR5-GFP homology donors, at several different AAV6 doses (MOIs). (C) Secretion of both IgM and IgG was detected, suggesting that cells had been successfully differentiated towards an antibody-secreting cell phenotype. (D) Stable GFP expression from primary human B cells electroporated with spCas9 RNPs targeting the CCR5 locus and matched AAV6-CCR5-GFP homology donor was measured after 8 days by flow cytometry.

FIG. 17 diagrams immunoglobulin locus rearrangement and antibody expression during B cell development, antigen encounter and memory development. In the germline configuration, there are 3 immunoglobulin loci, the heavy chain IgH locus and 2 distinct light chain loci, IgK and IgA. Each adopts a comparable configuration with a series of similar V segments, D segments (only in IgH) and J segments, followed by one or more constant regions. In the bone marrow, the Ig loci in developing B cells undergo sequential rearrangement at the DNA level. In IgH, the DNA of a randomly chosen D and J segment are brought into proximity and the intervening DNA is removed by the generation of double-stranded DNA breaks and ligated by NHEJ. Another step chooses a random V segment for similar rearrangement at the DNA level. During these rearrangements, NHEJ repair introduces indels that create additional variation at the sites of recombination known as junctional diversity (white). If this rearrangement in unsuccessful (i.e., out of frame) it can be attempted at the other allele. If recombination produces a functional product that can reach the cell surface by pairing with a surrogate light chain, rearrangement then occurs at either IgK or IgA. Following successful rearrangement of IgH and IgL, the B cell migrates to the spleen to finish maturing, after which it is known as a naïve B cell. Following antigen encounter, a B cell enters the germinal center to undergo additional evolution of the antibody response. Somatic hypermutation (yellow stars) is triggered by cytosine deamination of genomic DNA by the protein AID, which then recruits error-prone DNA repair pathways resulting in alteration of the coding sequence of the antibody gene. Antibodies that bind better to the antigen in the germinal center allow their host cell to proliferate, known as affinity maturation. Additionally, the local signaling milieu can trigger class switch recombination, whereby the heavy chain constant regions are rearranged at the DNA level. Antibody expression is driven by minimal promoter elements contained in the leader of each V segment that are dependent on the Ep enhancer. The VDJ segment that encodes for the VH domain is spliced to exons from the constant region gene that encodes for CH1, H, CH2, and CH3. Complex alternate splicing mechanism regulate the absence or addition of the transmembrane exons for secreted antibody or membrane BCR production, respectively.

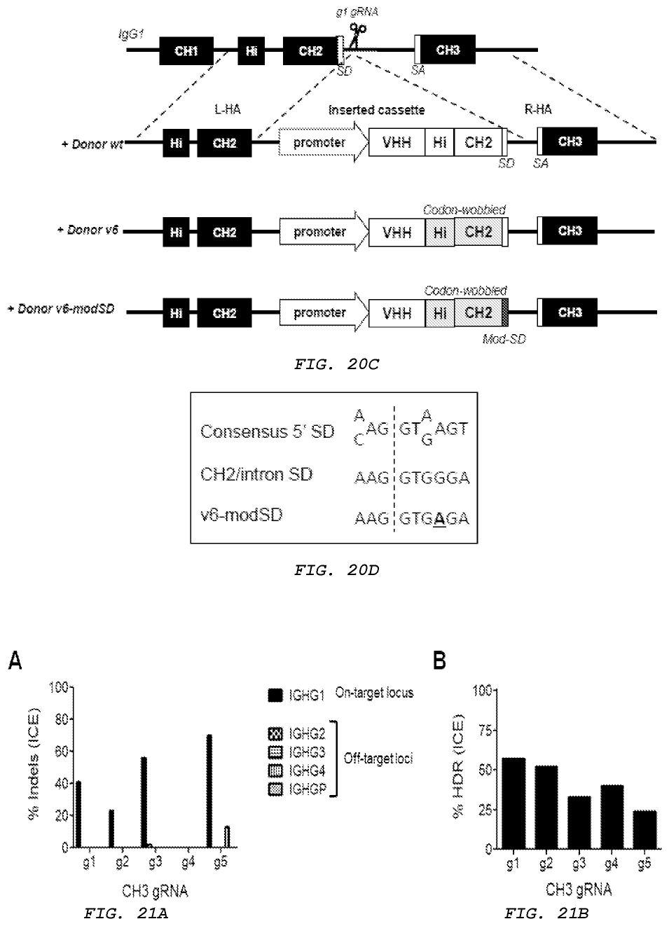

FIG. 18A-B demonstrates genome editing at the IGHG1 locus by inserting various alternate protein domains that bind to HIV gp120, as described in FIG. 2. Raji cells were genome edited using spCas9 RNPs comprising sg05, and corresponding plasmid homology donors, by nucleofection, as described in FIG. 10. Cell surface expression of the expected resulting single-chain constructs was detected by flow cytometry to detect IgG expression and binding to recombinant gp120. (A) PGT121 is a human anti-HIV bnAb and scFv cassettes were generated in both the heavy chain-light chain (HL) and light chain-heavy chain (LH) orientations using standard (G₄S)₃ linkers. CD4-mD1.22 is an engineered variant of domain 1 of CD4 that can bind to and neutralize HIV, but does not bind to MHC class II molecules. (B) A tandem bispecific sdAb was generated by inserting a tandem cassette of VHH-A6 and VHH-J3 joined by a (G₄S)₃ linker.

FIG. 19A-B provides a schematic of the strategy to express single (heavy) chain derived molecules, including single-domain antibodies and antibody-like molecules, by genome editing immunoglobulin heavy chain constant regions. (A) A simplified diagram of a germline human immunoglobulin heavy chain locus with V, D, and J genes, as well as the various downstream constant regions. Possible targets for genome editing include any of the Ig constant regions: IgM, IgD, IgG1-4, IgA1-2, or IgE. The most suitable target for a specific application will depend on the desired characteristics of the resulting molecule. (B) Within each constant region, any intronic region downstream of CH1 can be targeted for insertion of a functional expression cassette to generate a single (heavy) chain molecule, since omitting the CH1 exon renders the resulting molecule independent of a light chain. The IgG1 gene is shown here as an example, but this strategy applies to all constant region genes. By targeting different introns within a constant region, the size or functionality of the Fc region can be modulated. For example, the upstream exons can either be omitted entirely, or can be replaced by modified variants that are included in the cassette to be inserted. Further details describing the insertion of a promoter-VHH cassette downstream of CH1 are shown in FIG. 4, and an example showing insertion of a cassette downstream of CH2 is shown in FIG. 20.

FIG. 20A-D presents a schematic of genome editing at the IGHG1 locus by targeting the intron upstream of CH3. As an example, the use of a VHH domain is shown to create an sdAb, although other antibody or protein domains (e.g., other binding domains and related sequence), including those described in FIG. 2, could be used in place of the VHH domain. (A) Homology-directed repair (HDR), catalyzed by site-specific DNA double-stranded breaks produced by a targeted nuclease such as spCas9/gRNA promotes insertion of the indicated homology donor cassette in the intron upstream of CH3. In this example, the hinge and CH2 exons of the constant region are included in the inserted cassette, which therefore comprises a promoter (in these examples a B cell-specific EEK promoter), a functional domain (for example a VHH domain), the hinge and CH2 exons and a splice donor, and is flanked by sequences with homology to the Ig locus (homology arms). (B) Following HDR, the VHH cassette, hinge and modified CH2 sequences are inserted between the CH2 and CH3 exons of IgG1 in the human genome, as indicated. The inserted promoter drives transcription, and the splice donor after the inserted CH2 exon splices the resulting RNA transcript with the downstream genomic CH3 exon to produce the indicated single-chain antibody. Exclusion of the membrane exons M1 and M2 results in production of the secreted Ab, while their inclusion results instead in the transmembrane BCR. (C) Design of homology donor constructs to insert cassettes downstream of the CH2 exon in IgG1. Shown are the approximate location of the 750 bp left and right homology arms (HA) and the inserted cassettes, comprising in this example the EEK promoter and a J3 VHH antigen-binding domain fused to IgG1 hinge (Hi) and CH2 exons. Donors v6 and v6-modSD contain codon-wobbled Hinge-CH2 sequences to reduce homology with the endogenous Hinge-CH2 sequences. Donor v6-modSD also contains a modified splice donor (SD) at the junction of the CH2-downstream intron sequence. Additional modifications can be introduced into the CH2 sequence in the donor, for example to enhance ADCC activity. (D) Splice donor (SD) sequences, comparing the consensus sequence with the sequence at the junction of the native IgG1 CH2 exon and downstream intron. The SD in Donor v6-modSD was altered as indicated to better match the consensus.

FIG. 21A-B demonstrates genome editing at the intron upstream of CH3 in IGHG1 using spCas9 complexed with guide RNAs (gRNAs). (A) The activity of 5 spCas9 gRNAs (described in Table 4) targeting the intron upstream of the CH3 exon of IgG1 were assessed at the on-target IGHG1 gene site, as well as at 4 major predicted off-target regions (IGHG2, IGHG3, IGHG4, and IGHGP). Activity was measured by indel generation, which is one result after repair of DSBs, by Sanger sequencing (Hsiau et al. bioRxiv 2019 DOI: 10.1101/251082). On-target indels were observed for 4/5 guides. Moderate off-target activity was observed for CH3-g5 at IGHG4, and minor activity at IGHG2 for CH3-g3 was detected (limit of detection ˜2%). (B) Homology-directed repair (HDR) was measured using Sanger sequencing for all 5 gRNAs, following co-nucleofection of Cas9 RNPs and matched ssODN homology donors containing 40 bp homology arms on either side of the predicted Cas9 break site, to insert an XhoI restriction site. All 5 guides were able to support HDR (including CH3-g4 that did not exhibit detectable on-target indel formation), and CH3-g1 supported the highest HDR levels.

FIG. 22A-C shows evidence of somatic hypermutation occurring in a VHH-J3 sequence inserted at the IGHG1 locus by genome editing over time in Raji cells. (A) Mutagenesis of DNA in gene edited Raji B cells over time. Compared to the minimal mutagenesis in the input plasmid, increasing mutations were observed over time, particularly in CDR3, in the VHH-J3 sequence in the genome-edited Raji cells. In contrast, after 24 weeks, minimal mutagenesis was observed in the first 400 bp of a GFP sequence inserted into the same site in IGHG1 in Raji cells. (B) Graph showing the total percentage of the VHH-J3 sequence that was mutated (Total), the sum of the mutation frequencies for nucleotides within AID hotspot motifs (sequence: WRCH), and the sum of the mutation frequencies for cytosines within AID hotspots that are the target of AID activity. (C) Sequence logo plots of mutagenesis over time within CDR3. Arrows indicate locations of cytosines in AID hotspot motifs (SEQ ID NO:10).

FIG. 23A-E demonstrates the functional consequences of somatic hypermutation on VHH-J3 (SEQ ID NO:11). (A) Sequence logo of the population of translated sequences of CDR3 in VHH-J3, derived from the sequencing data in FIG. 22, demonstrates that somatic hypermutation can alter the coding sequence of the gene. (B) Protein sequences of NGS reads were classified based on the DNA sequence alterations observed after 24 weeks (ms: missense, reflecting the number of amino acid substitutions in the sequence). The majority of sequences at this point are expected to harbor changes to the CDR3 protein sequence. (C) Surface VHH-J3 expression in edited Raji cells was characterized over time by flow cytometry, showing that both the frequency of J3-expressing cells as well as the intensity of gp120 staining (MFI: median fluorescence intensity; a surrogate for affinity for HIV antigen) decreased over time. Note that the cells were cultured in the absence of any selection pressure to maintain or improve gp120 binding. (D) Total IgG secretion was quantified by ELISA from 500,000 engineered Raji cells after 2 days. The decline in total antibody secretion from an equal number of cells may reflect the impact of nonsense/frameshift mutations ablating protein translation in some cells, as also observed by surface staining. (E) The avidity of secreted VHH-J3 was quantified over time by gp120 ELISA. A dilution series containing normalized amounts of total IgG (quantified by ELISA) from each time point was used to measure absorbance at each point (left panel). The total absorbance sum was quantified (right panel), showing a significant decline in absorbance even at equal amounts of antibody. This suggests that, even among secreted antibody, somatic hypermutation caused a decline in the avidity of the antibody population and was functionally altering the antibodies. In an in vivo setting of a germinal center reaction, such somatic hypermutation would instead be expected to lead to affinity maturation rather than the decline in function we observed in vitro as a result of entropic mutagenesis in the absence of selective pressure.

FIG. 24A-J demonstrates genome editing, in vitro differentiation, and secretion of functional anti-HIV antibodies from primary human B cells engineered by insertion of the EEK/VHH-J3/splice donor cassette upstream of the hinge exon of IGHG1. B cells were transduced with AAV6 homology donors followed by electroporation with spCas9 RNPs containing sg05. (A) Diagram of AAV vector homology donor containing 750 bp homology arms (HA) flanking the target site of sg05, the B cell-specific EEK promoter, VHH-J3 sequence, and a splice donor. (B) Surface expression of VHH-J3 sdAb in untouched and genome edited cells after 8 days was measured by flow cytometry. (C) Primary B cells were subject to two different cell culture protocols: an expansion protocol using ImmunoCult™-ACF Human B Cell Expansion Supplement (Stem Cell Technologies) and a differentiation protocol adapted from Jourdan et al. (Blood 114: 5173-5181, 2009). The expansion protocol yielded robust (>200-fold) expansion over 11 days of culture, whereas minimal expansion was observed with the differentiation protocol. (D) In contrast, the differentiation protocol converted a significant portion of B cells into an antibody-secreting cell phenotype (CD20−CD27+CD38hi) relative to the expansion protocol. (E) ELISA was used to measure secretion of total IgG in the supernatant of cells treated with the indicated editing reagents and subject to the differentiation protocol. IgG concentrations were normalized by the number of viable cells and IgG secretion per cell increased over time in all populations, consistent with differentiation towards antibody-secreting phenotype. (F) RT-PCR of RNA from untouched or engineered cells at indicated days post-editing shows specific expression of VHH-J3 mRNA in engineered cells. While initially both the membrane and secreted splice isoforms are detected, as the cells are differentiated over time the membrane isoform is lost while the secreted form continues to be detected. This suggests that splicing of the chimeric antibody transgene is being regulated by the differentiation of the B cell, in the same way as occurs for an endogenous antibody. (G) HIV-specific human IgG detected by ELISA is only present in the supernatant from cells genome edited with both spCas9/gRNA and AAV6 homology donors (“genome edited”), with expression levels per cell tracking with the total IgG secretion per cell measured in panel (D). (H) Concentration-dependent neutralization of HIV infection was achieved using supernatants from genome edited cells (engineered supernatants), whereas no anti-HIV activity was present in supernatants from untouched cells or cells that received AAV6 only (controls). (I) IC50 values for HIV inhibition in supernatants from engineered B cells were calculated from HIV inhibition results shown in panel H. Shown are replicate experiments using engineered B cells from a single individual donor. The measured IC50 closely matches that previously determined for VHH-J3 produced by transient transfection of 293T cells. (J) Site-specific insertion of the VHH-J3 cassette was confirmed by in-out PCR to be only detected in the genomic DNA from cells that received both AAV6 and spCas9/gRNA.

FIG. 25 shows an example of a sequence (SEQ ID NO:12) of a VHH expression cassette suitable for insertion by genome editing at the intron between the CH1 and Hinge exons of human IGHG1. The sequence is annotated to show the following components: (1) a promoter, the EEK promoter (Luo et al. 2009 Blood 113:1422-1431), (2) one example of a DNA sequence that codes for the amino acids of VHH-J3 (McCoy et al. 2012 J Exp Med 209: 1091-1103) and (3) a splice donor (sd) sequence derived from the CH1 exon of IGHG1. The VHH-J3 sequence was reverse translated from the published amino acid sequence (McCoy et al. 2012 J Exp Med 209: 1091-1103), with the codons in the DNA sequence selected where possible to match the nearest human germline VH sequence (IGHV3-23D*01), as predicted by Ig BLAST (Ye et al. 2013 Nucleic Acids Res. 41: W34-W40). The complementarity determining regions (CDR1-3) in VHH-J3, as predicted by Ig BLAST, are underlined. Additionally, a leader sequence comprising a signal peptide from IGHV3-23D*01 was added in front of the VHH-J3 sequence. The italicized region downstream of the EEK promoter includes residual sequences from a multi-cloning site, and a Kozak sequence immediately preceding the ATG start codon of the IGHV3-23D*01 leader. Finally, the splice donor sequence was placed as indicated in order to promote correct splicing of the chimeric mRNA resulting from genome editing and thereby fuse the VHH-J3 domain with the downstream endogenous IGHG1 Hinge exon.

FIG. 26 shows the sequence (SEQ ID NO:13) of an example of a homology donor suitable for genome editing in order to insert a cassette at the intron between the CH1 and Hinge exons of human IGHG1. The donor sequence contains 500 bp (left and right) homology arms comprising sequences of the human IGHG1 gene on either side of the expected double-stranded DNA break point of the guide RNA IGHG1 Hinge-sg05 (predicted to be between base pairs −3 and −4 from the PAM sequence). The insertion cassette shown in this example contains VHH-J3 as an example, and is described in more detail in FIG. 25.

FIG. 27 shows an AAV homology donor genome (SEQ ID NO:14) suitable for genome editing and inserting a cassette at the intron between the CH1 and Hinge exons of human IGHG1. The AAV genome comprises AAV2 ITRs, 750 bp (left and right) homology arms suitable for use with guide RNA IGHG1 Hinge-sg05 and an expression cassette for VHH-J3 as an example. The homology arms can each independently be various lengths (e.g., 100 bp to 1000 bp). The expression cassette is described in more detail in FIG. 25.

FIG. 28A-F shows optimizing homology donors for editing and expression following insertion at the IgG1 CH2-CH3 intron. (A) A plasmid expression construct mimicking the final gene-edited configuration was used to compare expression of sdAbs containing the J3 VHH fused to either the wildtype (wt) IgG1 Hinge-CH2 sequence, or 6 different codon-wobbled sequences. Relative expression levels were measured by IgG1 ELISA of supernatants harvested from transiently transfected 293T cells. Construct v6 gave optimal expression, that was 2-fold higher than the wildtype sequence. (B) Plasmid homology donors were constructed containing either the wildtype or v6 Hinge-CH2 sequences, as shown in FIG. 20C, and electroporated into Raji cells together with Cas9 RNPs containing the CH3-g1 gRNA (see also Table 4). Correct gene-edited events were identified using a specific nested in-out PCR, and were only detected for the sample containing donor v6. (C) Expression of gp120-binding sdAbs on the surface of the edited Raji cells was evaluated using flow cytometry; however, no expression was detected, for either the wildtype or v6 donors. (D) Using the same construct and assay as in (A), it was found that modifying the native 5′ SD at the CH2-intron boundary enhanced expression from the plasmid mimicking the final gene-edited configuration using a donor containing the modified v6 sequence. (E) Raji cells were edited with CH3-g1 gRNA/Cas9 RNPs and AAV6 vectors containing the v6-modSD donor sequence cassette. gp120-binding sdAbs were detected on the cell surface by flow cytometry, indicating successful editing and expression with the v6-modSD donor. (F) Supernatants from the control or edited Raji cells were harvested and used in an HIV neutralization assay on Tzmb1 cells, confirming activity of secreted engineered sdAbs.

FIG. 29 shows edited B cells respond to antigen immunization. Human tonsil organoids were established as described in Wager et al., Nature Medicine, 2021, with the addition of less than 1% of control or edited B cells. The cultures were immunized with a mixture of gp120 protein and the Adju-Phos adjuvant. Twelve days later, cells were stained for expression of edited sdAbs (IgG+ cells and bind to gp120). Addition of B cells edited to express J3 sdAbs, using either the CH1-hinge intron insertion site (CH1-Hi) or the CH2-CH3 intron insertion site (CH2-CH3) editing strategies, resulted in a significant increase in gp120-specific B cells in response to the gp120 immunization protocol. A significantly lower background of gp120-responsive cells was also observed in the control (no edited B cells) population, and was expected, due to the activation of naturally gp120-reactive B cells in such cultures.

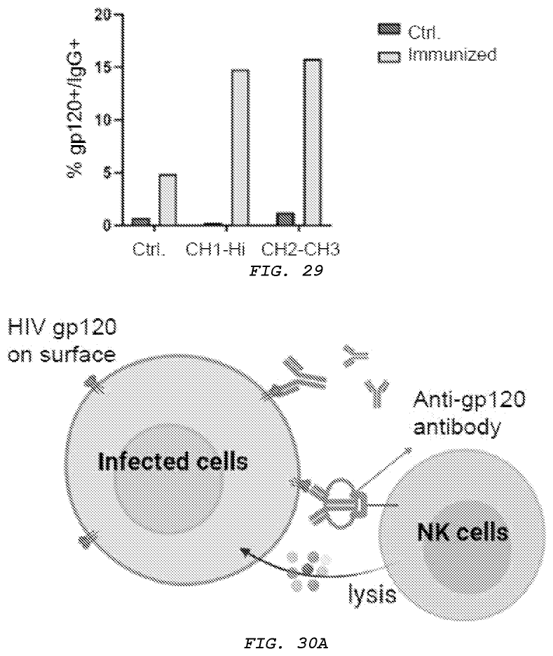

FIG. 30A-B shows the introduction of GASDALIE mutations into the CH2 sequence of the J3 sdAb enhances ADCC activity. (A) ADCC assay. NL4-3 strain HIV-infected CEM.NKR.CCR5 cells (target cells) were stained with CellTrace dye and mixed with anti-gp120 antibodies and NK92 CD16+NK cells, for 4 hours. ADCC activity was measured as % HIV-infected cells in the live target cell population. (B) ADCC activity was measured using a positive control broadly neutralizing anti-gp120 antibody, 3BNC117 (3B), with known ADCC activity against NL4-3, and a negative control antibody PGT121 (PG), which does not have ADCC activity against NL4-3. The J3 sdAbs evaluated contained the native IgG1 CH2 domain, or a modified CH2 sequence containing the GASDALIE mutations (J3++), which enhanced ADCC activity, or the negative control LALA mutations (J3−−), which ablated ADCC activity.

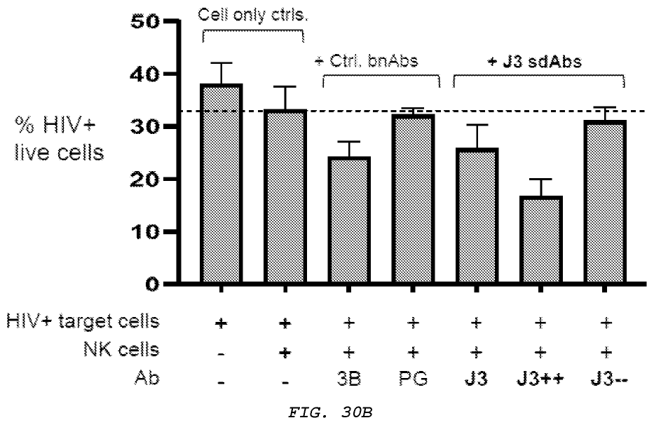

FIG. 31 shows the sequence (SEQ ID NO: 34) of an example of a homology donor suitable for genome editing to insert a sequence at the intron between the CH2 and CH3 exons of human IGHG1. The donor sequence contains 750 bp homology arms (left and right) comprising sequences of the human IGHG1 gene on either side of the expected double-stranded DNA point of the guide RNA IGHG1 CH3-g1. The insertion cassette shown in this example comprises the EEK promoter, VHH-J3, and a DNA sequence corresponding to the amino acid sequences of the IgG1 Hinge and CH2 sequences. The Hinge and CH2 sequences are codon wobbled compared to the native sequence to reduce homology and prevent interference with the intended homology-directed event. The splice donor (boxed) has been modified to enhance expression of the engineered sdAb. The insertion cassette is named donor v6-modSD and is also described in FIG. 1.

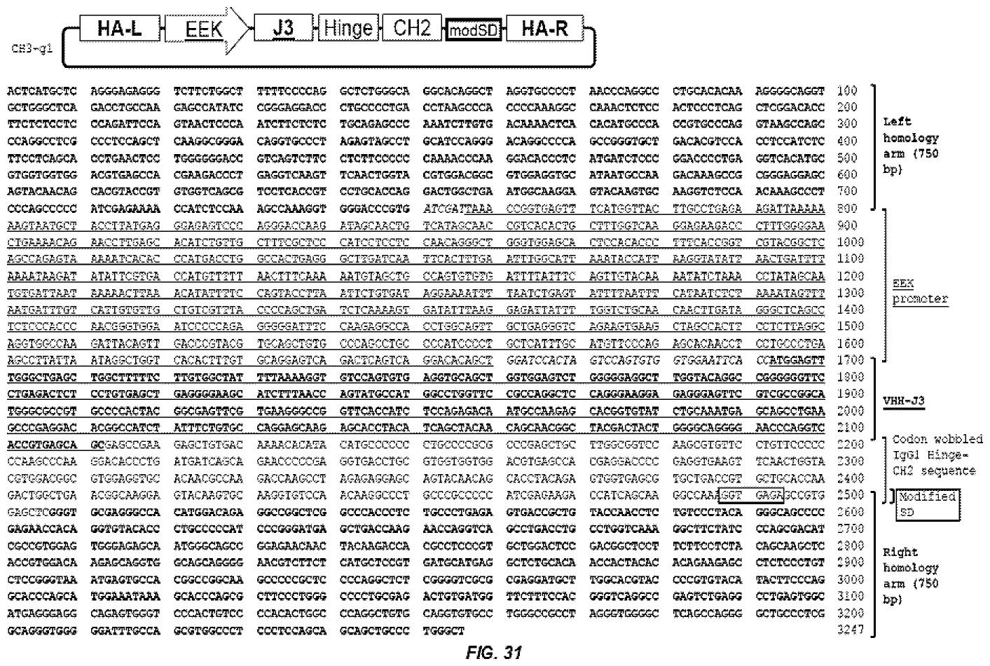

FIG. 32 shows alternate sequences comprising amino acid changes can also be introduced into the donor Hinge or CH2 sequences to alter the sdAb properties. In this example (SEQ ID NO: 35), the CH2 sequence has been further modified to introduce the GASDALIE (SEQ ID NO:36) mutations to enhance ADCC properties (italicized and underlined in CH2).

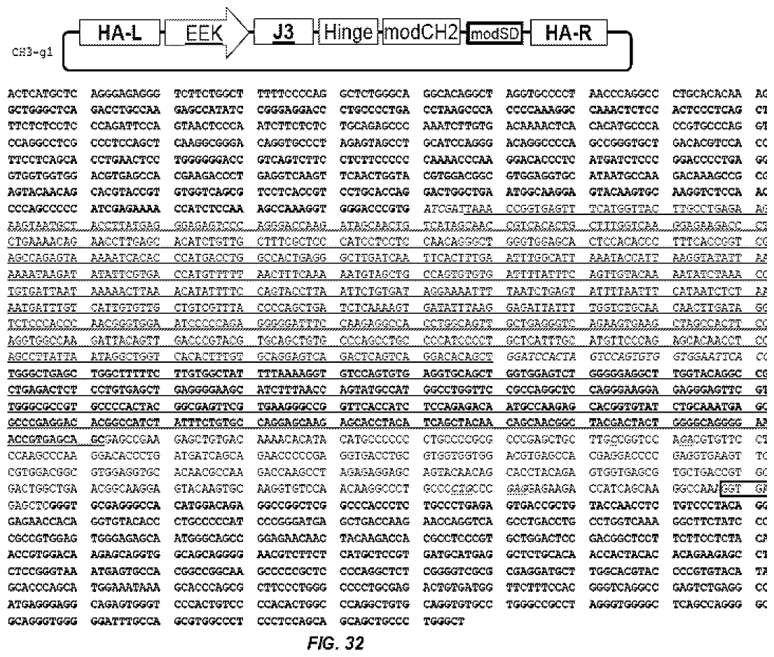

FIG. 33A-B shows editing IgG4 to replace the entire antibody sequence. (A) Design of homology donor construct to insert cassettes at the end of the CH3 exon in IgG4. This design allows replacement of the entire antibody sequence, including CH3. Shown are the approximate locations of left and right homology arms (HA) and the inserted cassette, which contains codon-wobbled Hinge-CH2-CH3 sequences to reduce homology with the endogenous Hinge-CH2-CH3 sequences. Additional modifications can be introduced into the constant region sequences in the donor, for example to enhance antibody half-life or ADCC activity. (B) Cutting efficiency (% indels) at on-site target for the indicated guide RNAs targeting the end of the CH3 exon in IgG4. Raji cells were electroporated with RNPs containing Cas9 protein complexed with each gRNA, and indels were measured by ICE assay. No off-target indels were detected at other IgG loci. Sequences are shown in Table 6. HDR editing efficiency for each indicated gRNA was measured using donor sequences comprising single-stranded oligonucleotide homology donors containing a 6 bp restriction site.

FIG. 34A-B shows CrossMab designs that prevent heterologous antibody chain pairings. (A) Expression of 2 different antibodies (endogenous and engineered) in the same cell has the potential for heterologous pairings between the H and L chains of the 2 different antibodies, as in in these two examples. (B) In contrast to normal H+L chain antibodies, CH1-CL CrossMabs swap the positions of the CH1 and CL domains, retaining the wanted H+L pairings while minimizing the potential for heterologous mispairings with conventional H and L chains in the same cell.

FIG. 35A-C provide examples of expression cassette designs to produce full-length H+L antibodies and CrossMab antibodies. In each case, site-specific insertion of the cassette produces a construct where splicing occurs from the splice donor at the C-terminal exon of the cassette to the downstream constant region gene, producing a full-length H+L chain antibody using a 2A ribosome skipping motif to separate the chains. (A) Using conventional antibody chain components. (B and C) Using a CH1-CL CrossMab design. Constructs A and B would be inserted downstream of the CH1 exon (as shown in FIG. 37), and construct C would be inserted downstream of the CH2 exon (as shown in FIG. 20). Constant region sequences (CL, CH1, Hi, CH2) are codon wobbled to reduce homology with the endogenous sequences, as previously described.

FIG. 36A-D show gene editing strategies to express full-length (H+L) CrossMab. (A) Homology donor design to direct CH1-CL CrossMab expression from IgG1, following repair of DNA break introduced by CH3-g1 gRNA and Cas9. The indicated cassette is inserted between CH2 and CH3 and comprises in this example the VL and VH domains from Rituximab, and using a 2A ribosome skipping motif to separate the chains. An HA tag was added to the C-terminal end of CH1 to facilitate antibody identification. (B) Flow cytometry showing surface expression of the CH1-CL CrossMab after genome editing. (C) In-out (junctional) PCR confirmed site-specific gene insertion in the edited cells. Editing was also confirmed by Sanger sequencing (not shown). (D) RT-PCR analysis of mRNA confirmed expression of the CH1-CL CrossMab construct only in edited cells, in both secreted (left) and membrane-anchored (right) splice isoforms.

FIG. 37 shows the specific sequence of the homology donor used to generate the example of the Rituximab CrossMab (SEQ ID NO:42).

### DETAILED DESCRIPTION

As used herein and in the appended claims, the singular forms “a,” “an,” and “the” include plural referents unless the context clearly dictates otherwise. Thus, for example, reference to “an immune cell” includes a plurality of such immune cells and reference to “the single chain antibody” includes reference to single chain antibodies and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless otherwise defined, all technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7. All headings and subheading provided herein are solely for ease of reading and should not be construed to limit the invention. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and specific examples are illustrative only and not intended to be limiting. All publications mentioned herein are incorporated herein by reference in full for the purpose of describing and disclosing the methodologies, which might be used in connection with the description herein. Moreover, with respect to any term that is presented in one or more publications that is similar to, or identical with, a term that has been expressly defined in this disclosure, the definition of the term as expressly provided in this disclosure will control in all respects.

It should be understood that this invention is not limited to the particular methodology, protocols, and reagents, etc., described herein and as such may vary. The terminology used herein is for the purpose of describing particular embodiments only and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to describe the present invention, in connection with percentages means±1%.

The term “adeno-associated virus” or “AAV” as used herein refers to a member of the class of viruses associated with this name and belonging to the genus dependoparvovirus, family Parvoviridae. Multiple serotypes of this virus are known to be suitable for gene delivery; all known serotypes can infect cells from various tissue types. At least 11, sequentially numbered, are disclosed in the prior art. Non-limiting exemplary serotypes useful for the purposes disclosed herein include any of the 11 serotypes, e.g., AAV2 and AAV6. The term “lentivirus” as used herein refers to a member of the class of viruses associated with this name and belonging to the genus lentivirus, family Retroviridae. While some lentiviruses are known to cause diseases, other lentivirus are known to be suitable for gene delivery. See, e.g., Tomás et al. (2013) Biochemistry, Genetics and Molecular Biology: “Gene Therapy—Tools and Potential Applications,” ISBN 978-953-51-1014-9, DOI: 10.5772/52534.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity. The “class” of an antibody refers to the type of constant domain or constant region possessed by its heavy chain. There are five major classes of antibodies: IgA, IgD, IgE, IgG and IgM, and several of these can be further divided into subclasses (isotypes), e.g., IgG₁, IgG₂, IgG₃, IgG₄, IgA₁, and IgA₂. The heavy chain constant domains that correspond to the different classes of immunoglobulins are called a, δ, ε, γ, and μ, respectively. The light chain of an antibody can be assigned to one of two types, called kappa (κ) and lambda (λ), based on the amino acid sequence of its constant domain.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, Fab′h, Fv fragments, scFv antibody fragments, disulfide-linked Fvs (sdFv), a Fd fragment consisting of the VH and CH1 domains, linear antibodies, single domain antibodies such as sdAb (either vL or vH), camelid vHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide mini bodies).

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the disclosure includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

Non-limiting examples of target antigens include: antigens associated with infectious agents including, but are not limited to proteins, glycoproteins (e.g., surface or coat proteins of bacteria or viruses), mixtures of proteins (e.g., bacterial cell lysate), other detectable compounds associated with an infectious agent or particles (e.g., virus-like particles or viral coat proteins, bacterial surface antigens, etc.); CD3, CD5, CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors, a glycosylated CD43 epitope expressed on non-hematopoietic cancers, Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ES0-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p⁵³ mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of lmprinted Sites), Squamous Cell Carcinoma Antigen Recognized By T Cells 3 (SART3); Paired box protein Pax-5 (PAX5); proacrosin binding protein sp32 (OY-TESl); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl), MPL, Biotin, c-MYC epitope Tag, CD34, LAMP1 TROP2, GFRalpha4, CDH17, CDH6, NYBR1, CDH19, CD200R, Slea (CA19.9; Sialyl Lewis Antigen); Fucosyl-GM1, PTK7, gpNMB, CDH1-CD324, DLL3, CD276/B7H3, IL11Ra, IL13Ra2, CD179b-IGLl1, TCRgamma-delta, NKG2D, CD32 (FCGR2A), Tn ag, Timl-/HVCR1, CSF2RA (GM-CSFR-alpha), TGFbetaR2, Lews Ag, TCR-beta1 chain, TCR-beta2 chain, TCR-gamma chain, TCR-delta chain, FITC, Leutenizing hormone receptor (LHR), Follicle stimulating hormone receptor (FSHR), Gonadotropin Hormone receptor (CGHR or GR), CCR4, GD3, SLAMF6, SLAMF4, HIV1 envelope glycoprotein, HTLV1-Tax, CMV pp65, EBV-EBNA3c, KSHV K8.1, KSHV-gH, influenza A hemagglutinin (HA), GAD, PDL1, Guanylyl cyclase C (GCC), auto antibody to desmoglein 3 (Dsg3), auto antibody to desmoglein 1 (Dsg1), HLA, HLA-A, HLA-A2, HLA-B, HLA-C, HLA-DP, HLA-DM, HLA-DOA, HLA-DOB, HLA-DQ, HLA-DR, HLA-G, IgE, CD99, Ras G12V, Tissue Factor 1 (TF1), AFP, GPRC5D, Claudin18.2 (CLD18A2 or CLDN18A.2)), P-glycoprotein, STEAP1, Liv1, Nectin-4, Cripto, gpA33, BST1/CD157, low conductance chloride channel, and the antigen recognized by TNT antibody. In a particular embodiment, the first VHH fragment has specificity to a tumor antigen. In a particular embodiment, the tumor antigen is selected from CEA, EGFR, Her2, EpCAM, CD20, CD30, CD33, CD47, CD52, CD133, CEA, gpA33, Mucins, TAG-72, CIX, PSMA, folate-binding protein, GD2, GD3, GM2, VEGF, VEGFR, Integrin, o/β3, α5β1, ERBB2, ERBB3, MET, IGF1 R, EPHA3, TRAILR1, TRAILR2, RANKL, FAP and Tenascin.

As used herein “affinity” is meant to describe a measure of binding strength. Affinity, in some instances, depends on the closeness of stereochemical fit between a binding agent and its target (e.g., between an antibody and antigen including epitopes specific for the binding domain), on the size of the area of contact between them, and on the distribution of charged and hydrophobic groups. Affinity generally refers to the “ability” of the binding agent to bind its target. There are numerous ways used in the art to measure “affinity”. For example, methods for calculating the affinity of an antibody for an antigen are known in the art, including use of binding experiments to calculate affinity. Binding affinity may be determined using various techniques known in the art, for example, surface plasmon resonance, bio-layer interferometry, dual polarization interferometry, static light scattering, dynamic light scattering, isothermal titration calorimetry, ELISA, analytical ultracentrifugation, and flow cytometry. An exemplary method for determining binding affinity employs surface plasmon resonance. Surface plasmon resonance is an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.).

As used herein an “antigen recognition cassette” comprises a polynucleotide encoding a binding domain that binds to a desired target (e.g., an antigen) linked to a splice donor sequence and driven by a regulatory element such as a promoter.

As used herein, the term “binding domain” refers to a domain or portion of a larger molecule that has a binding specificity for a second molecule and binds to that second molecule with an affinity higher than a non-specific domain. Binding domains are present in antibody and antibody fragments as well as on certain receptors and other molecules (e.g., non-immunoglobulin binding scaffolds). Typically a molecule that has a binding domain is a protein, e.g., an immunoglobulin chain or fragment thereof, comprising at least one domain, e.g., immunoglobulin variable domain sequence that can bind to a target with affinity higher than a non-specific domain. The term encompasses antibodies and antibody fragments. In another embodiment, an antibody molecule is a multispecific antibody molecule, e.g., it comprises a plurality of immunoglobulin variable domain sequences (a plurality of binding domains), wherein a first immunoglobulin variable domain sequence of the plurality has binding specificity for a first epitope and a second immunoglobulin variable domain sequence of the plurality has binding specificity for a second epitope. In another embodiment, a multispecific antibody molecule is a bispecific antibody molecule. A bispecific antibody has specificity for two antigens. A bispecific antibody molecule is characterized by a first immunoglobulin variable domain sequence which has binding specificity for a first epitope and a second immunoglobulin variable domain sequence that has binding specificity for a second epitope.

“Cancer” and “cancerous” refer to or describe the physiological condition in mammals that is typically characterized by unregulated cell growth. Examples of cancer include, but are not limited to B-cell lymphomas (Hodgkin's lymphomas and/or non-Hodgkins lymphomas), T cell lymphomas, myeloma, myelodysplastic syndrome, skin cancer, brain tumor, breast cancer, colon cancer, rectal cancer, esophageal cancer, anal cancer, cancer of unknown primary site, endocrine cancer, testicular cancer, lung cancer, hepatocellular cancer, gastric cancer, pancreatic cancer, cervical cancer, ovarian cancer, liver cancer, bladder cancer, cancer of the urinary tract, cancer of reproductive organs thyroid cancer, renal cancer, carcinoma, melanoma, head and neck cancer, brain cancer (e.g., glioblastoma multiforme), prostate cancer, including but not limited to androgen-dependent prostate cancer and androgen-independent prostate cancer, and leukemia. Other cancer and cell proliferative disorders will be readily recognized in the art. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

The term “Cas9” refers to a CRISPR-associated, RNA-guided endonuclease such as Streptococcus pyogenes Cas9 (spCas9) and orthologs and biological equivalents thereof. Biological equivalents of Cas9 include but are not limited to C2c1 from Alicyclobacillus acideterrestris and Cpf1 (which performs cutting functions analogous to Cas9) from various bacterial species including Acidaminococcus spp. and Francisella novicida U112. Cas9 may refer to an endonuclease that causes double stranded breaks in DNA, a nickase variant such as a RuvC or HNH mutant that causes a single stranded break in DNA, as well as other variations such as deadCas-9 or dCas9, which lack endonuclease activity. Cas9 may also refer to “split-Cas9” in which CAs9 is split into two halves—C-Cas9 and N-Cas9—and fused with a two intein moieties. See, e.g., U.S. Pat. No. 9,074,199 B1; Zetsche et al. (2015) Nat Biotechnol. 33(2):139-42; Wright et al. (2015) PNAS 112(10) 2984-89. Non-limiting examples of commercially available sources of SpCas9 comprising plasmids can be found under the following AddGene reference numbers:
42230: PX330; SpCas9 and single guide RNA
48138: PX458; SpCas9-2A-EGFP and single guide RNA
62988: PX459; SpCas9-2A-Puro and single guide RNA
48873: PX460; SpCas9n (D10A nickase) and single guide RNA
48140: PX461; SpCas9n-2A-EGFP (D10A nickase) and single guide RNA
62987: PX462; SpCas9n-2A-Puro (D10A nickase) and single guide RNA
48137: PX165; SpCas9

As used herein, the term “complementary” when used in reference to a polynucleotide is intended to mean a polynucleotide that includes a nucleotide sequence capable of selectively annealing to an identifying region of a target polynucleotide under certain conditions. As used herein, the term “substantially complementary” and grammatical equivalents is intended to mean a polynucleotide that includes a nucleotide sequence capable of specifically annealing to an identifying region of a target polynucleotide under certain conditions. Annealing refers to the nucleotide base-pairing interaction of one nucleic acid with another nucleic acid that results in the formation of a duplex, triplex, or other higher-ordered structure. The primary interaction is typically nucleotide base specific, e.g., A:T, A:U, and G:C, by Watson-Crick and Hoogsteen-type hydrogen bonding. In certain embodiments, base-stacking and hydrophobic interactions can also contribute to duplex stability. Conditions under which a polynucleotide anneals to complementary or substantially complementary regions of target nucleic acids are well known in the art, e.g., as described in Nucleic Acid Hybridization, A Practical Approach, Hames and Higgins, eds., IRL Press, Washington, D.C. (1985) and Wetmur and Davidson, Mol. Biol. 31:349 (1968). Annealing conditions will depend upon the particular application, and can be routinely determined by persons skilled in the art, without undue experimentation.

As used herein, the term “CRISPR” refers to Clustered Regularly Interspaced Short Palindromic Repeats (CRISPR). CRISPR may also refer to a technique or system of sequence-specific genetic manipulation relying on the CRISPR pathway. A CRISPR recombinant expression system can be programmed to cleave a target polynucleotide using a CRISPR endonuclease and a guide RNA. A CRISPR system can be used to cause double stranded or single stranded breaks in a target polynucleotide. A CRISPR system can also be used to recruit proteins or label a target polynucleotide. In some aspects, CRISPR-mediated gene editing utilizes the pathways of nonhomologous end-joining (NHEJ) or homologous recombination to perform the edits. These applications of CRISPR technology are known and widely practiced in the art. See, e.g., U.S. Pat. No. 8,697,359 and Hsu et al. (2014) Cell 156(6): 1262-1278. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). In addition to expression of the Cas9 nuclease, the CRISPR-Cas9 system uses an RNA molecule to recruit and direct the nuclease activity to target polynucleotide sequence of interest. These guide RNAs (gRNAs) take one of two forms: (i) a synthetic or expressed trans-activating CRISPR RNA (tracrRNA) plus a CRISPR RNA (crRNA) designed to cleave the gene target site of interest and (ii) a synthetic or expressed single guide RNA (sgRNA) that consists of both the crRNA and tracrRNA as a single construct. The crRNA and the tracrRNA form a complex which acts as the guide RNA for the Cas9 enzyme. The scaffolding ability of tracrRNA along with crRNA specificity can be combined into a single synthetic gRNA which simplifies guiding of gene alterations to a one component system which can increase efficiencies.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

The terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, the term “functional” may be used to modify any molecule, biological, or cellular material to intend that it accomplishes a particular, specified effect.

The term “gRNA” or “guide RNA” as used herein refers to the guide RNA sequences used to target specific genes for correction employing the CRISPR technique. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRIPSPR RNA (tracrRNA); or a polynucleotide comprising CRISPR RNA (crRNA) and trans-activating CRIPSPR RNA (tracrRNA). In some aspects, a gRNA is synthetic (Kelley, M. et al. (2016) J of Biotechnology 233 (2016) 74-83). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing). CRISPR/Cas9 strategies can employ a vector to transfect the mammalian cell. The guide RNA (gRNA) can be designed for each application as this is the sequence that Cas9 uses to identify and directly bind to the target DNA in a cell. Multiple crRNAs and the tracrRNA can be packaged together to form a single-guide RNA (sgRNA). The sgRNA can be joined together with the Cas9 gene and made into a vector in order to be transfected into cells. The disclosure provides gRNAs comprising SEQ ID Nos: 15-33, wherein T is replaced with U.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

The term “non-immune binding scaffolds” or “non-immune synthetic binding molecules” refer to molecules that have antigen binding domains, but differ in structure to that of an antibody and can be generated either from nucleic acids, as in the case of aptamers, or from non-immunoglobulin protein scaffolds/peptide aptamers, into which hypervariable loops are inserted to form the antigen binding domain. Constraining the hypervariable binding loop at both ends within the protein scaffold improves the binding affinity and specificity of the non-immunoglobulin binding domains to levels comparable to or exceeding that of a natural antibody. One advantage of these molecules compared to use of the typical antibody structure is that they have a smaller size.

As used herein, the term “operably linked” refers to the relationship between a first reference nucleotide sequence (e.g., a gene or coding sequence) and a second nucleotide sequence (e.g., a regulatory element) that allows the second nucleotide sequence to affect one or more properties associated with the first reference nucleotide sequence (e.g., a transcription rate). In the context of the disclosure, a regulatory element is operably linked to a coding sequence (e.g., a binding domain coding sequence) when the regulatory element is positioned within a vector such that it exerts an effect (e.g., a promotive or tissue-selective effect) on transcription of the coding sequence.

The term “ortholog” is used in reference of another gene or protein and intends a homolog of said gene or protein that evolved from the same ancestral source. Orthologs may or may not retain the same function as the gene or protein to which they are orthologous. Non-limiting examples of Cas9 orthologs include S. aureus Cas9 (“saCas9”), S. thermophiles Cas9, L. pneumophilia Cas9, N. lactamica Cas9, N. meningitides Cas9, B. longum Cas9, A. muciniphila Cas9, and O. laneus Cas9.

The term “polynucleotide”, “nucleic acid”, or “recombinant nucleic acid” refers to polymers of nucleotides such as deoxyribonucleic acid (DNA), and, where appropriate, ribonucleic acid (RNA).

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors. Non-limiting exemplary promoters include CMV promoter and U6 promoter. Generally, promoter elements are located 5′ of the translation start site of a coding sequence or gene. However, in certain embodiments, a promoter element may be located within an intron sequence, or 3′ of the coding sequence. In some embodiments, a promoter useful for a genetic engineering is derived from a native gene of the target protein (e.g., a Factor VIII promoter). In some embodiments, a promoter is specific for expression in a particular cell or tissue of the target organism (e.g., a liver-specific promoter). In yet other embodiments, one of a plurality of well characterized promoter elements is used. Non-limiting examples of well-characterized promoter elements include the CMV early promoter, the R-actin promoter, and the methyl CpG binding protein 2 (MeCP2) promoter. In some embodiments, the promoter is a constitutive promoter, which drives substantially constant expression of an operably linked coding sequence. In other embodiments, the promoter is an inducible promoter, which drives expression of an operably linked coding sequence in response to a particular stimulus (e.g., exposure to a particular treatment or agent). For a review of designing promoters for AAV-mediated gene therapy, see Gray et al. (Human Gene Therapy 22:1143-53 (2011)), the contents of which are expressly incorporated by reference in their entirety for all purposes.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the term “regulatory elements” refers to nucleotide sequences, such as promoters, enhancers, terminators, polyadenylation sequences, IRESs, introns, etc., that provide for the expression of a coding sequence in a cell.

The term “scFv” refers to a protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “subject” is intended to include living organisms that can be modified by the methods and compositions of the disclosure.

“TALEN” refers to an enzyme that can cleave specific sequences in a DNA molecule. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011)).

The term “therapeutic effect” refers to a biological effect which can be manifested by various means, including but not limited to, e.g., decrease in tumor volume, a decrease in the number of cancer cells, a decrease in the number of metastases, an increase in life expectancy, decrease in cancer cell proliferation, decrease in cancer cell survival, decrease in the titer of the infectious agent, a decrease in colony counts of the infectious agent, amelioration of various physiological symptoms associated with a disease condition.

“Treatment” and “treating,” as used herein refer to both therapeutic treatment and prophylactic or preventative measures, wherein the object is to prevent or slow down (lessen) the targeted pathologic condition, prevent the pathologic condition, pursue or obtain beneficial results, or lower the chances of the individual developing the condition even if the treatment is ultimately unsuccessful. Those in need of treatment include those already with the condition as well as those prone to have the condition or those in whom the condition is to be prevented.

As used herein, “zinc-finger nucleases” or “ZFNs” refer to artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms.

Antibodies are naturally generated in developing B cells through a complex process involving recombination and mutagenesis of common starting sequences present in the immunoglobulin locus (Ig) locus. This process results in a vast repertoire of antibodies with different specificities, poised to respond to antigens present, for example, on foreign infectious agents. Once created by this process, a specific antibody variant will be displayed on the surface of a B cell in the form of a B cell receptor (BCR). Engagement of the BCR with a corresponding antigen leads to activation of that specific B cell, resulting in expansion, maturation and the secretion of its specific antibody. The antibody repertoire in the body is thus available for the selection and amplification of extremely specific responses. Additionally, B cell responses evolve over time, and generate antibody-secreting descendants that are capable of surviving and producing antibodies for decades, as well as memory responses that can be recalled upon antigen re-encounter.

Antibodies are naturally generated in developing B cells through a complex process involving recombination and mutagenesis of common starting sequences present in the immunoglobulin locus (Ig) locus. This process results in a vast repertoire of antibodies with different specificities, poised to respond to antigens present, for example, on foreign infectious agents. Once created by this process, a specific antibody variant will be displayed on the surface of a B cell in the form of a B cell receptor (BCR). Engagement of the BCR with a corresponding antigen leads to activation of that specific B cell, resulting in expansion, maturation and the secretion of its specific antibody. The antibody repertoire in the body is thus available for the selection and amplification of extremely specific responses. Additionally, B cell responses evolve over time, and generate antibody-secreting descendants that are capable of surviving and producing antibodies for decades, as well as memory responses that can be recalled upon antigen re-encounter.

In addition to this natural process that selects antibodies that recognize a specific antigen, “pre-formed” antibodies with desirable properties can be used as recombinant protein drugs, for example to treat cancer, infectious diseases, and autoimmune diseases. This approach can provide passive immunization, as well as allowing the use of antibodies with properties that may not efficiently form in nature. An example of the latter case are the so-called ‘broadly neutralizing’ antibodies (bnAbs) directed against the human immunodeficiency virus (HIV). bnAbs are rare antibodies that can inhibit many different strains of HIV but are often highly evolved and do not form easily during natural infections or in response to vaccinations. However, their ability to broadly recognize many different strains of HIV means that they are desirable for use as both a prevention strategy and a therapy.

In addition to the delivery of recombinant antibody proteins, antibody therapies are also being developed based on gene therapy approaches. Here, the desired antibody gene can be delivered as a self-contained expression cassette using, for example, AAV vectors. The engineered cells then produce and secrete the therapeutic antibody.

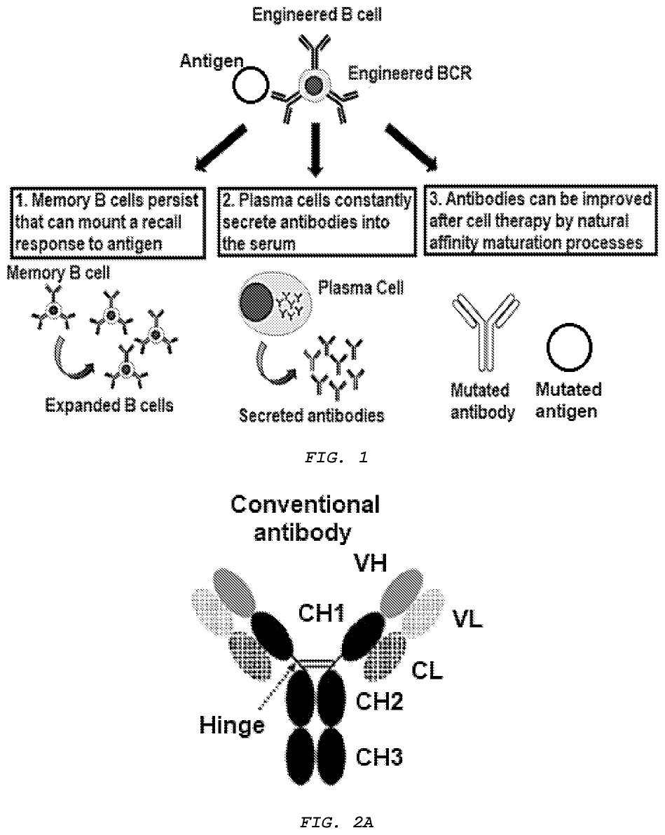

By inserting an antigen recognition cassette at the natural Ig locus, two important and highly desirable features of the immune response are preserved: (1) the ability to respond to the presence of an antigen, resulting in continuous production of the antibody without the need for constant re-infusions of expensive recombinant antibodies and (2) the ability for the antibody to mutate through defined cellular processes and potentially evolve alongside the disease, to further prevent the development of resistance to the therapy (see FIG. 1). These properties are currently not possible when secreting an antibody from a non-natural cell, such as muscle, or when the antibody is expressed from a non-Ig locus.

Provided herein is an innovative genome engineering strategy that provides for the production of antibody fragments (e.g., single chain, single domain antibodies and the like) and non-immunoglobulin binding molecules from a specific locus (e.g., human Ig locus) of an immune cell (e.g., human B cell) or B cell precursors (e.g., hematopoietic stem cells, induced stem cells, embryonic stem cells and the like). The genome engineering techniques, methods and compositions described herein can be performed on autologous cells to a subject in need of treatment as well as allogeneic cells. The method can be performed ex vivo or in vivo.

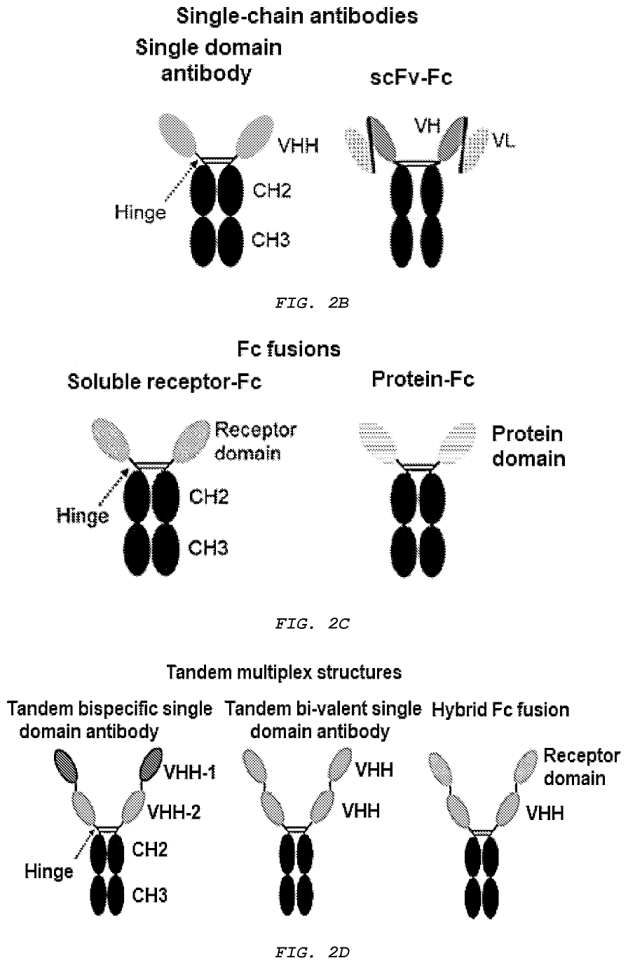

For example, the disclosure shows that sdAbs can be generated from engineered B cells. sdAbs can be recombinantly produced and are a unique type of antibody produced by camelids that are of a much simpler design than standard human antibodies. sdAbs comprise only the equivalent of a heavy chain rather than the normal combination of heavy and light chains (e.g., see FIG. 2). The sdAb heavy chain comprises an antigen-binding VHH domain and a constant region which comprises Hinge, CH2 and CH3 domains. sdAbs, however, lack the CH1 domain that is used for light chain pairing in conventional two chain antibodies. In this way, sdAbs differ from the H chain of conventional antibodies because they are able to be expressed despite the lack of an L chain partner. Further, camelid VHH domains are homologous to the VH3 family of human heavy chain variable region (VH) segments, and are capable of forming functional sdAb antibodies when grafted onto human IgG H chain scaffolds lacking the CH1 domain. In this way “humanized” sdAbs can be generated and have been used as antibody drugs against HIV, influenza virus, rotavirus, MERS coronavirus, and breast cancer. Moreover, additional humanization of the framework regions can Attomey docket No. 00130-034US1 further enhance the homology of VHH sequences to human antibodies to levels comparable to currently marketed humanized monoclonal antibodies. The first product based on VHH technology was approved by the FDA in February of 2019.

sdAbs do not contain the CH1 domain of the heavy chain. In addition to being required for H chain+L chain pairing, the CH1 domain also regulates antibody secretion, adopting a disordered structure that prevents secretion of free H chain unless it is paired with an L chain. Thus, sdAbs are incompatible with the CH1 exon and cannot be produced by using the strategies described above.

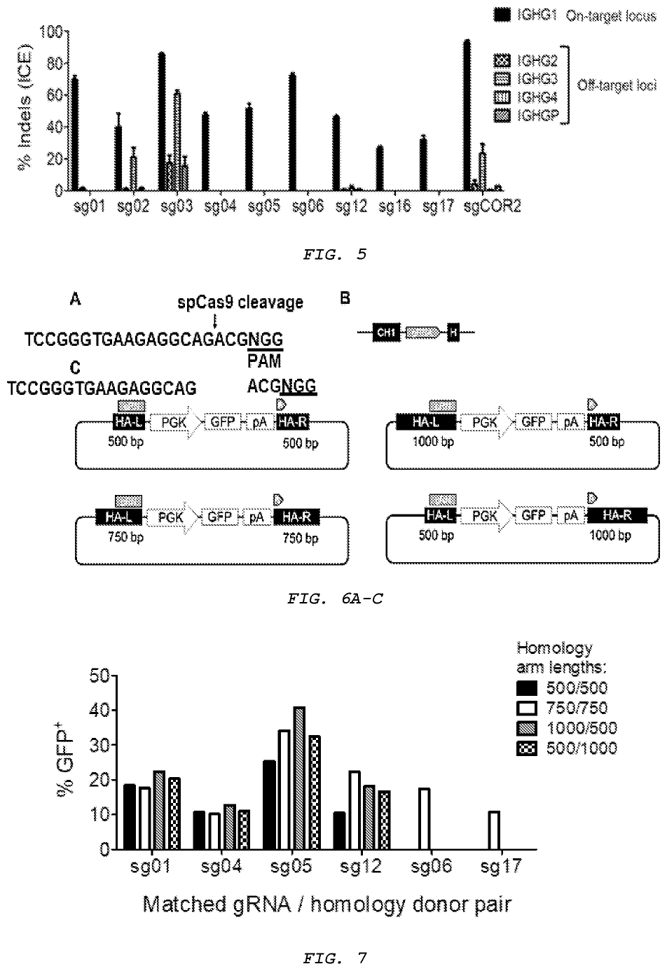

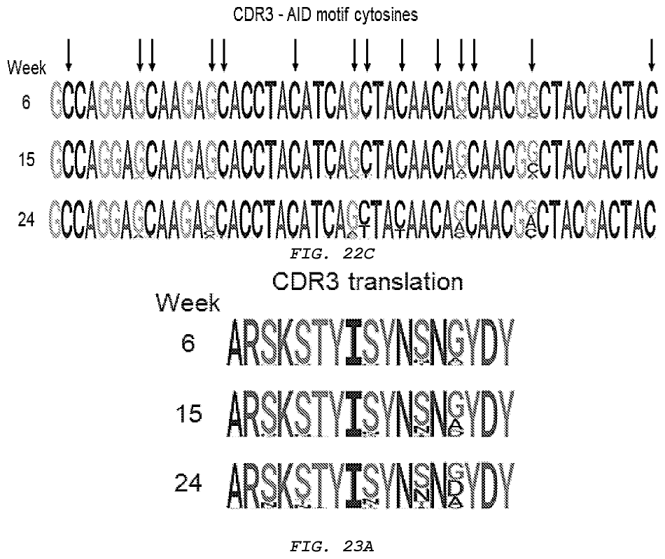

In a particular embodiment, an antigen-binding VHH domain is inserted into an Ig constant region gene downstream of the CH1 exon, with gene expression driven by an internal promoter (see FIG. 4; see also FIG. 19 for other sites of insertion). While the ability of the inserted antibody to undergo class-switch recombination is improbable, alternative splicing of downstream exons will still drive production of both the secreted antibody and the transmembrane B cell receptor (BCR) necessary for antigen-specific B cell function. It should be noted that the studies presented herein have performed gene editing at the IGHG1 locus (i.e., IgG1), but clearly the genome engineering strategies of the disclosure can be applied other loci as well. The IGHG1 locus was chosen for the studies, as it is a prevalent subclass of IgG and possesses effector functions, such as the ability to trigger ADCC, which are important in anti-HIV applications. For example, the genome engineering strategies of the disclosure can be also be applied to generate antibody fragments (e.g., sdAbs, scFv etc.) and non-immunoglobulin binding domain from other IgG subclasses (IgG2-4) or from other antibody classes (IgM, IgD, IgA, or IgE), thereby producing antibody fragments (e.g., sdAbs, scFv etc.) and non-immunoglobulin binding domain with different effector functions.

In addition, the use of antibody fragments (e.g., sdAbs, scFv etc.) and non-immunoglobulin binding domain of the disclosure could support the creation of multiplex antibody-like constructs that simultaneously recognize different antigen targets (e.g., see FIG. 2D). These could include different sites on a virus such as HIV, which would reduce the ability of the virus to evolve resistance to a single antibody, or multiple antigens expressed on a cancer cell, similarly reducing the likelihood of escape mutations developing. Such multiplexed antibody fragments (e.g., sdAbs, scFv etc.) and non-immunoglobulin binding domain can be generated using the genome engineering strategies presented herein. In a particular embodiment, the disclosure provides for a tandem multi-specific sdAbs. A tandem bispecific sdAbs comprises 2, 3, 4 or more antigen-binding domains linked in tandem, where each antigen-binding domain binds to a different antigen. Examples of making such tandem multi-specific antibodies are described in Alvarez-Cienfuegos et al., (“Intramolecular trimerization, a novel strategy for making multispecific antibodies with controlled orientation of the antigen binding domains” Scientific Reports 6: 28643 (2016))

In a particular embodiment, genome editing technologies (e.g., CRISPR/Cas9) are used to introduce an antigen recognition cassette into an immunoglobulin (Ig) locus within an immune cell (e.g., a B cell, or a B cell precursor for example a hematopoietic stem cell (HSC) or induced pluripotent stem cell). The approaches described herein have the added advantage that a natural antibody producing cell type (e.g., a B cell) can be used as to produce the antibody fragments (e.g., sdAbs, scFv etc.) and non-immunoglobulin binding domain of the disclosure. Additionally, by editing the natural Ig locus, two important and highly desirable features of the immune response are preserved: (1) the potential for ongoing evolution of the antibody alongside the disease, to enhance the affinity of the antibody for its antigen and to further prevent the development of resistance to the therapy; and (2) the ability to respond to antigen, resulting in continuous production of the antibodies without the need for constant reinfusions of expensive recombinant antibodies. These properties are currently not possible when secreting an antibody from a non-natural cell, such as muscle cell, and when the antibody is expressed from a non-Ig locus.

In a certain embodiment, single domain antibodies (sdAbs) are produced by the genome editing strategies presented herein. sdAbs are a unique type of antibody produced by camels/llamas that are of a much simpler design than standard antibodies, comprising only one protein chain rather than the normal combination of heavy and light chains.

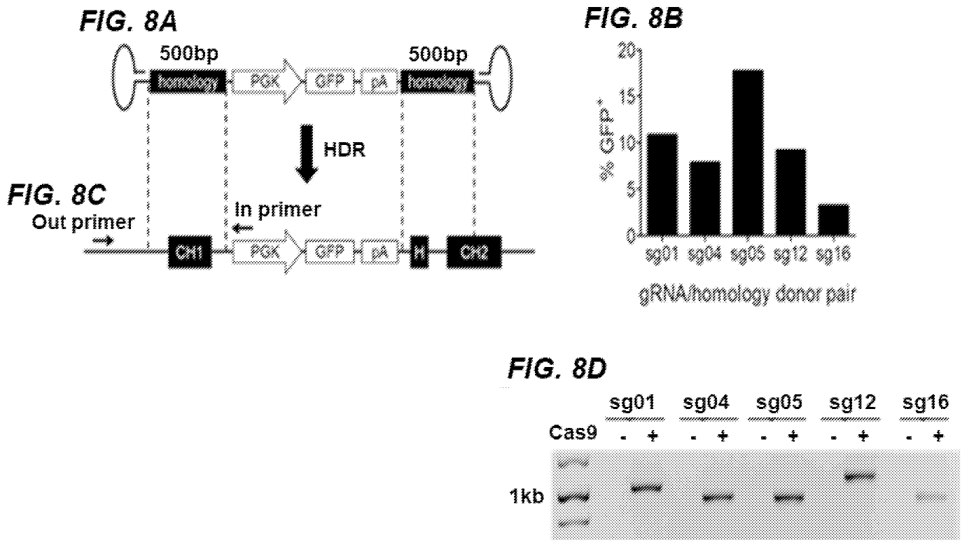

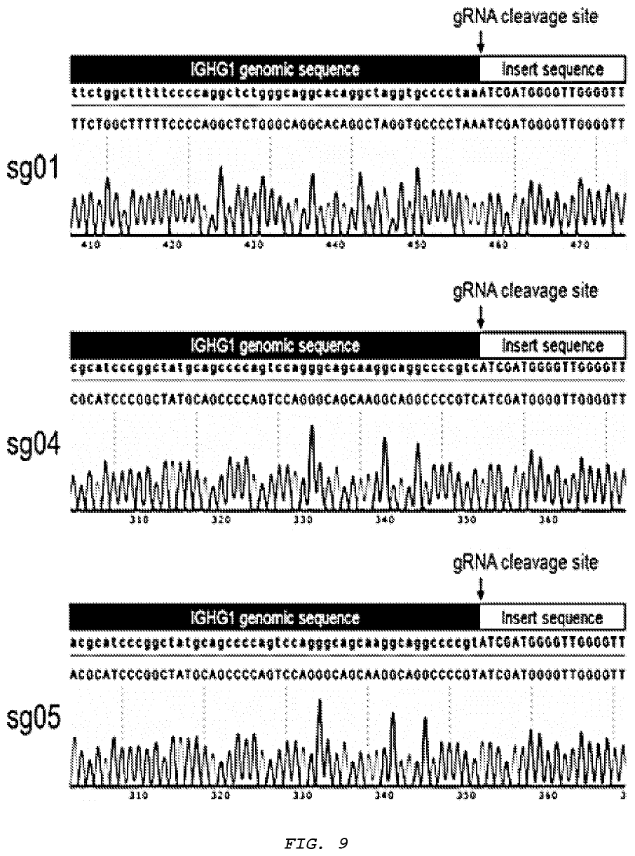

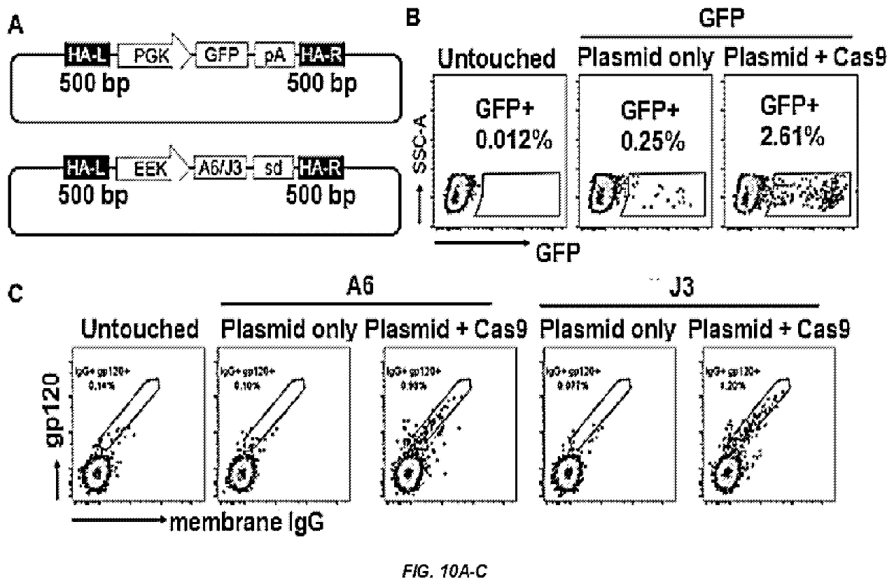

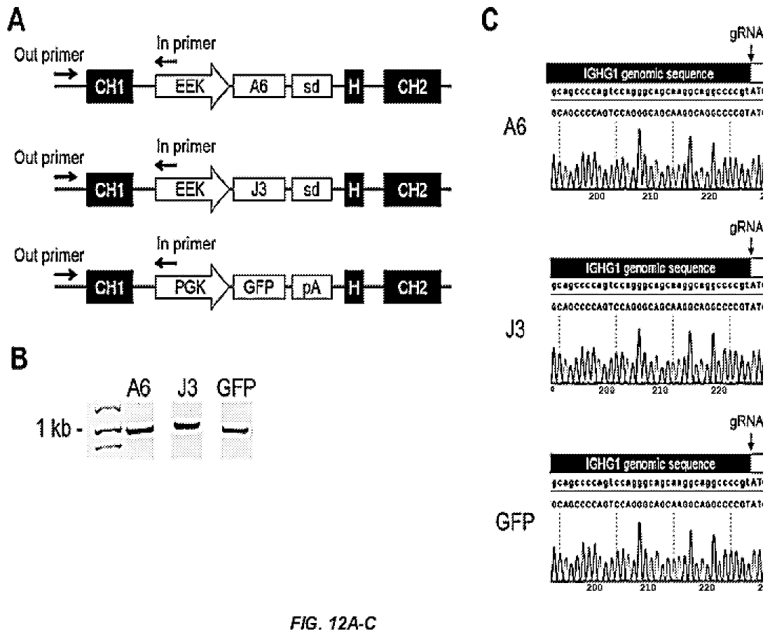

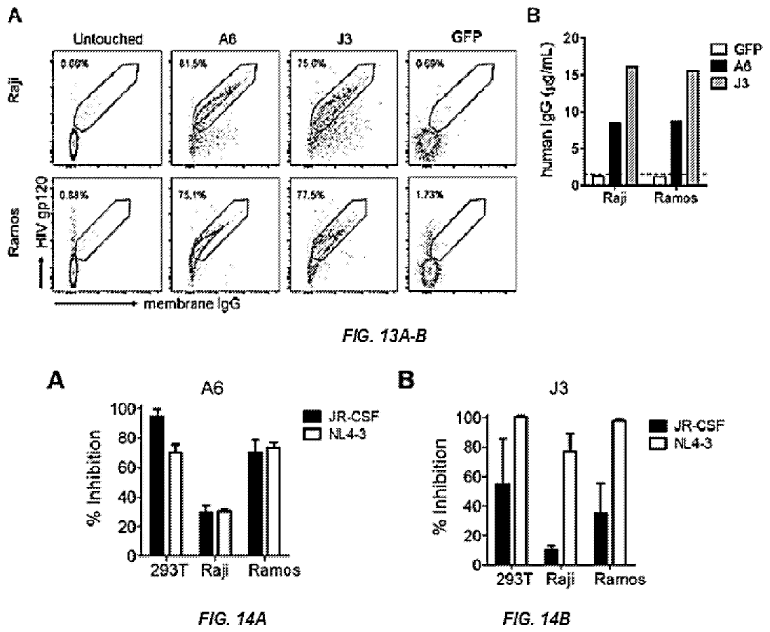

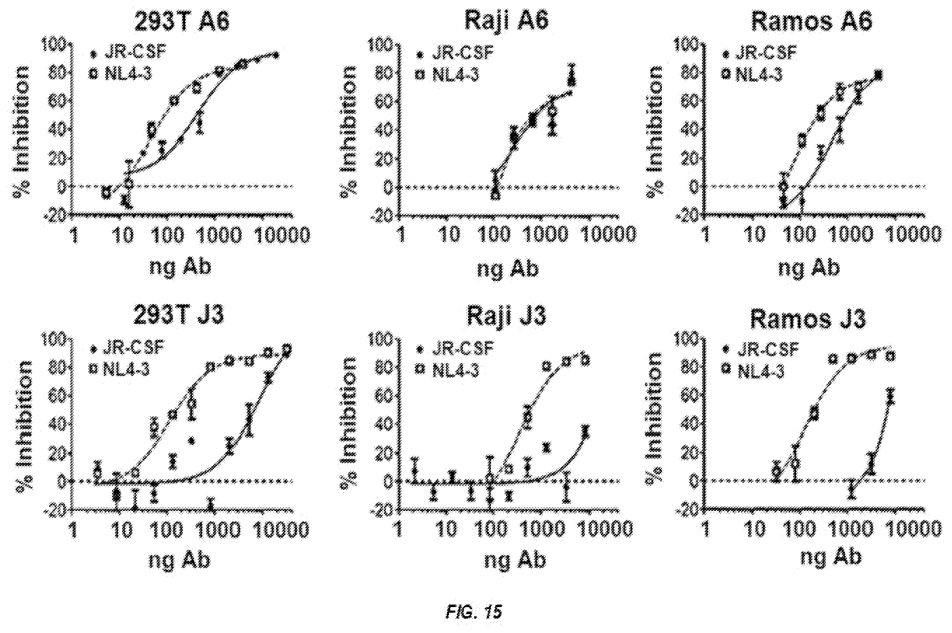

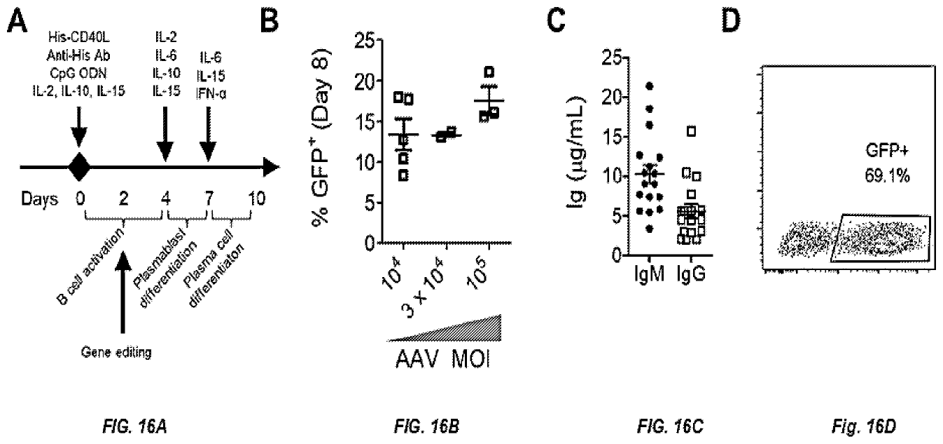

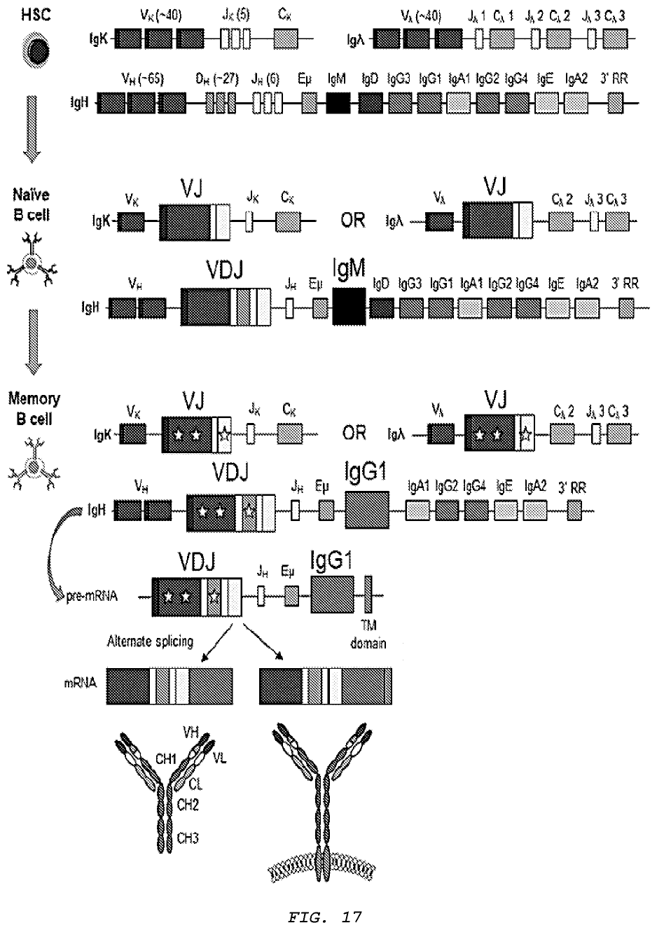

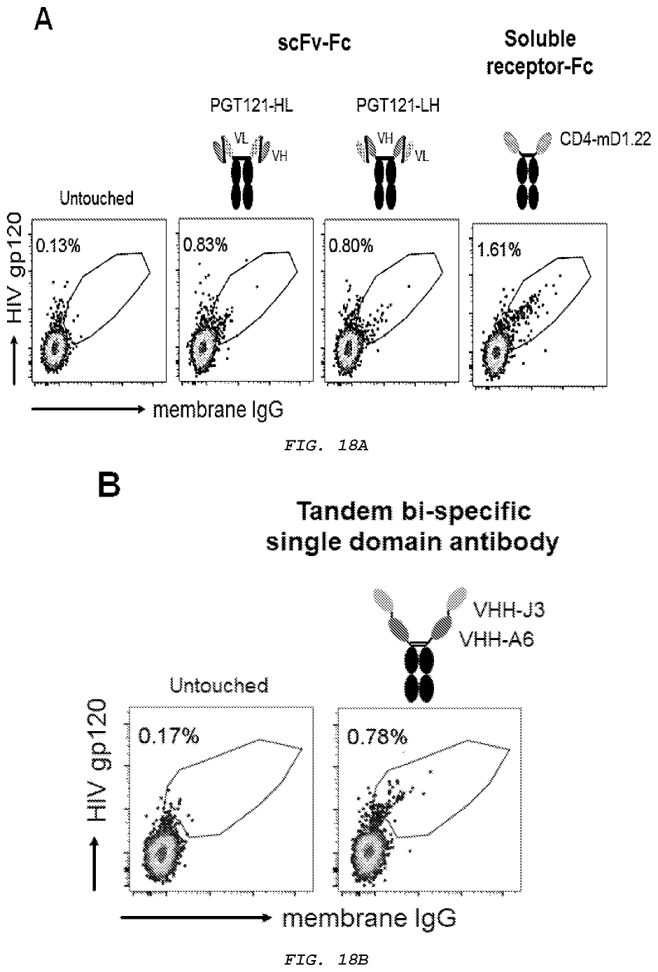

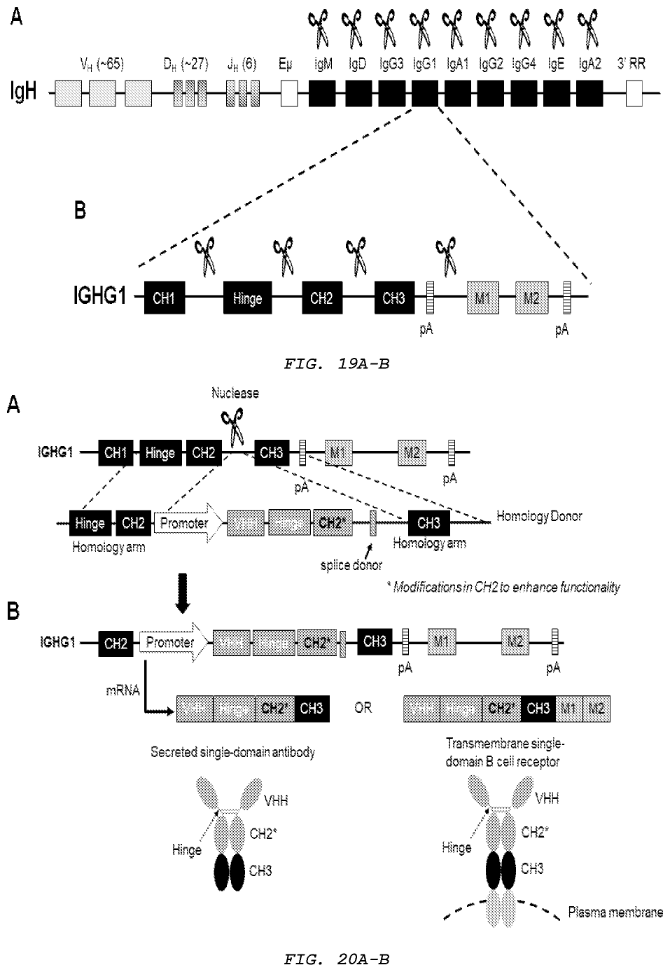

As described in the studies presented herein, guide RNAs were designed to introduce a DNA break into the human IgG1 locus at a specific site, but which had no detectable off-target activity at homologous IgG sequences (e.g., see FIG. 5). A series of homology donor cassette were evaluated in different vector systems. For example, a plasmid vector was used in K562 cells, while an AAV6 vector was used with B cells or K562 cells (e.g., see FIGS. 6-8, and 23). Confirmation of site-specific genome editing was determined by using in-out PCR and Sanger sequencing analyses (e.g., see FIGS. 8-9, and 12). In the examples, the sdAbs produced by the genome editing approaches described herein retained functionality as they were: (1) able to be expressed on the cell surface of B cells and bind anti-IgG antibodies and the HIV Env gp120 protein (e.g., see FIGS. 10-11); (2) be secreted as antibodies into cell culture supernatants (e.g., see FIG. 13); and (3) neutralize both X4 and R5-tropic strains of HIV with a similar profile as when the sdAbs were produced from a non-integrated plasmid expression cassette (e.g., see FIGS. 14-15 and Table 5). Additionally, the disclosure provides methods to engineer primary human B cells, so that the primary B cells can be differentiated in vitro; and to detect secretion of both IgM and class-switched IgG antibodies during B cell differentiation (e.g., see FIG. 16).

Accordingly, the genome engineering strategies described herein can be used to produce recombinant Ig antibody fragments (e.g., sdAbs, scFv etc.) and non-immunoglobulin binding domain expressed from a human Ig locus. Further, both secreted antibody fragments (e.g., sdAbs) and B cell receptors (BCRs) can be produced using the genome engineering strategies disclosed herein. One advantage of the genome engineering strategies of the disclosure is that ‘engineered’ B cells can be produced, which can continually produce desired antibody fragments (e.g., sdAbs, scFv etc.) and non-immunoglobulin binding domains in vivo. In contrast, current antibody therapies are administered as recombinant proteins that must be isolated from cells or microorganisms. To produce the recombinant proteins at scale requires use of expensive reactor systems which must provide a sterile environment to propagate cells, and further the recombinant proteins have to be isolated from the cells at a high purity, requiring the use of expensive purification equipment. It is not overly surprising that some of these recombinant protein therapies cost upwards of $100,000/dose. As a protein, these therapies have a limited half-life, requiring frequent re-administration if prolonged activity of the treatment is required.

The approach of engineering H+L chain antibodies within the Ig constant region domains has the advantage that monoclonal antibodies can be produced with defined Ig isotypes, thereby controlling functionalities. For instance, IgG1 or IgG3 editing could produce antibodies with desired effector functions such as ADCC, IgG4 editing could minimize such effector functions to generate primarily binding antibodies, and IgA editing could enhance functionality at mucosal surfaces such as the gut or lungs. This approach also bypasses the need to force class-switching in cells after editing, either ex vivo through cytokine treatments or in vivo through vaccine/adjuvant design or route of administration, both of which are relatively poorly understood in human B cells. Editing with a defined isotype would also be expected to improve the lot-to-lot consistency of a cell therapy and be advantageous from a clinical manufacturing perspective.

Modifications of the H+L design to minimize erroneous cross-pairing (e.g., CrossMab) also provides advantages of safety and reduces complexity of editing. In this embodiment, additional modification or knockout of the endogenous H and/or L chain loci are not required. Because the CrossMab design uses replacement of the CH1 domain, this method uses gene insertion downstream of the CH1 exon and is possible using the editing approach described for the native Ig locus, e.g., by inserting into constant regions downstream of the CH1 exon.

Co-expression of an endogenous and engineered antibody could also present additional functional opportunities. For instance, by editing antigen-specific B cells, it would be possible to have dual-functional B cells able to target either 2 different antigens, or 2 different targets on the same antigen. The former could provide opportunities to, for example, compensate for immunodeficiencies by expressing natural pathogen-specific antibodies alongside the therapeutic antibody. The latter could be useful in the case of mutagenic viral infections such as HIV or SARS-CoV-2, by targeting multiple epitopes to prevent escape mutagenesis from the therapy.

In a further embodiment, these approaches could be combined with the previously detailed modifications of the Fc stalk to enhance or modulate antibody effector functions, such as ADCC, or half-life, and the like.

Antigen-specific B cells following antigen encounter can survive for decades in vivo, remaining primed for expansion upon antigen re-encounter as well as continuing to produce protective antibodies from long-lived plasma cells. Accordingly, using the genome engineering strategies of the disclosure can provide for an ‘engineered’ B-cell with a synthetic immunoglobulin locus, but which retains normal functionality and effector functions, and further provides a prolonged therapeutic or prophylactic benefit which could last for the lifetime of the patient. As the ‘engineered’ B cells would be antigen specific, the therapy should be capable of self-tuning, boosting itself as needed without complex monitoring of patients or medical interventions needed to maintain activity within a therapeutic window. Moreover, B cells can naturally evolve antibody specificity over time through a process known as affinity maturation. By performing the genome engineering strategies of the disclosure at the endogenous immunoglobulin locus, it is expected that ‘engineered’ B cells will also be capable of applying these natural processes to the synthetic gene introduced through gene editing. Further, ‘engineered’ B cells can travel to relevant sites of infection or disease in the body to secrete functional antibodies. As such, ‘engineered’ B cells can access sites normally protected from parts of the immune system (such as B cell follicles in HIV infection); can achieve therapeutic efficacy at much lower doses than systemic delivery of recombinant proteins; avoid potential side effects (e.g., systemic immunosuppression in autoimmunity) or off-target effects (e.g., damaging or killing healthy cells throughout the body with anti-cancer antibodies whose target might be weakly expressed on other cells).

The genome engineering strategies described herein can be used to produce antibody fragments (e.g., sdAbs) and non-immunoglobulin binding domains from a human Ig locus. A normal human antibody is generated from two separate genes, a heavy and a light chain, which must then associate within the cell after protein synthesis prior to secretion. Thus, replicating full specificity of an antibody within a B cell would require introduction of both of these sequences into a cell. Since the heavy and light chains are located on different chromosomes, engineering fully natural antibody specificity would require editing at both of these loci. Performing sequential manipulations of the two loci would greatly increase the cost and complexity of the procedure. Some investigators have begun to explore gene editing of the immunoglobulin locus using synthetic transgenic constructs that express both the heavy and light chain from a single site in the genome. These rely on endogenous enhancer activity to drive a minimal antibody promoter element, as well as a 2A ribosome skipping motif to get protein translation of both the light chain and the heavy chain. These motifs are rarely 100% effective, and having weak activity could result in single chain antibodies with greatly reduced functionality or even introduce toxicity to the producer cell.

In contrast, the engineered antibody fragments (e.g., sdAbs, scFv etc.) and non-immunoglobulin binding domains of the disclosure contain all of their specificity within a single sequence, and do not require engineering at multiple loci. Furthermore, the nature of antibody fragments (e.g., single domain antibodies) and non-immunoglobulin binding domains allows editing at an alternative site in the IgH locus, with desirable properties (more consistent homology than other strategies). Further, the genome editing strategies to produce such antibody fragments (e.g., sdAbs, scFv etc.) and non-immunoglobulin binding domains described herein avoid safety issues seen with other genome editing procedures at the immunoglobulin locus. For example, investigators have recently began to show that, with gene editing tools based on double-stranded break generation, performing simultaneous manipulations at 2 or more locations can greatly increase genotoxic risks of DNA rearrangements such as inversions or translocations that could lead to cell death, dysfunction, or generation of cells that could be more likely to become cancerous in the future.

The genome editing strategies of the disclosure can be used to produce, for example, sdAbs, which contain multiple antigen recognition domains. Single domain antibodies are particularly amenable for engineering of constructs containing multiple antigen recognition domains. This approach has been previously demonstrated for recombinant proteins with single domain antibodies against influenza. Combining multiple recognition domains in a sdAb can increase sdAb efficacy in a variety of ways, including, but not limited to, increasing sdAb avidity so that it is more likely to bind to the target; making the sdAb more resistant to mutations by the infectious agent or tumor to avoid immune detection; providing for multiple effector functions, including but not limited to engagement of NK cell-mediated killing with an anti-CD16 domain, or recruiting T cell effector functions through an anti-CD3 domain.

The antibody fragments (e.g., sdAbs, scFv etc.) and non-immunoglobulin binding domains of the disclosure have reduced immunoreactivity than other protein-based therapies. Recombinant antibodies, even when fully humanized, come with the risk of anti-drug antibodies developing that are directed against the idiotype, and that can both prevent therapeutic efficacy and lead to adverse reactions. Current strategies to achieve long-term expression of antibodies against infectious diseases such as HIV through gene therapy (AAV vectors delivering antibody genes to muscle cells) have been hampered by extremely high rates of host antibodies directed against the therapeutic antibody. This is likely due to the known immunogenic nature of muscle-directed gene transfer with adeno-associated viral vectors that has been employed in non-human primates and in humans for this approach. Within the host, anti-idiotypic antibodies do not frequently prevent antibody function, suggesting that B cells have intrinsic tolerogenic mechanisms to prevent these deleterious immune reactivities. Additionally, a number of studies have used retroviral-based gene transfer in murine B cells to express proteins or peptides fused to antibody sequences and shown that these modified cells can induce active immune tolerance to the foreign protein by serving as tolerogenic antigen-presenting cells. It is postulated herein, that similar mechanisms will function for the ‘engineered’ B cells of the disclosure, allowing long-term production of therapeutic single domain antibodies without adverse immune reactions by the host.

In a particular embodiment, antibody fragments (e.g., sdAbs, scFv etc.) and non-immunoglobulin binding domains made by a method the disclosure can be used to treat a disease cause by an infectious agent by binding to antigens associated with the infectious agent. In a further embodiment, the infectious agent is a virus, a bacterium, a fungus, a parasitic helminth, or a parasitic protozoan. Examples of viruses include, but are not limited to those in the following virus families: Retroviridae (for example, human immunodeficiency virus (HIV), human T-cell leukemia viruses; Picornaviridae (for example, poliovirus, hepatitis A virus, enteroviruses, human coxsackie viruses, rhinoviruses, echoviruses, foot-and-mouth disease virus); Caliciviridae (such as strains that cause gastroenteritis, including Norwalk virus); Togaviridae (for example, alphaviruses (including chikungunya virus, equine encephalitis viruses, Simliki Forest virus, Sindbis virus, Ross River virus, rubella viruses); Flaviridae (for example, hepatitis C virus, equine non-primate hepaci virus (NPHV), dengue viruses, yellow fever viruses, West Nile virus, Zika virus, St. Louis encephalitis virus, Japanese encephalitis virus, Powassan virus and other encephalitis viruses); Coronaviridae (for example, coronaviruses, severe acute respiratory syndrome (SARS) virus, Middle East respiratory syndrome (MERS) virus; Rhabdoviridae (for example, vesicular stomatitis viruses, rabies viruses); Filoviridae (for example, Ebola virus, Marburg virus); Paramyxoviridae (for example, parainfluenza viruses, mumps virus, measles virus, respiratory syncytial virus); Orthomyxoviridae (for example, influenza viruses); Bunyaviridae (for example, Hantaan viruses, Sin Nombre virus, Rift Valley fever virus, bunya viruses, phleboviruses and Nairo viruses); Arenaviridae (such as Lassa fever virus and other hemorrhagic fever viruses, Machupo virus, Junin virus); Reoviridae (e.g., reoviruses, orbiviurses, rotaviruses); Birnaviridae; Hepadnaviridae (hepatitis B virus); Parvoviridae (parvoviruses); Papovaviridae (papilloma viruses, polyoma viruses, BK-virus); Adenoviridae (adenoviruses); Herpesviridae (herpes simplex virus (HSV)-1 and HSV-2; cytomegalovirus; Epstein-Barr virus; varicella zoster virus; Kaposi's sarcoma herpesvirus (KSHV); and other herpes viruses, including HSV-6); Poxviridae (variola viruses, vaccinia viruses, pox viruses); and Iridoviridae (such as African swine fever virus); Astroviridae; and unclassified viruses (for example, the etiological agents of spongiform encephalopathies, the agent of delta hepatitis (thought to be a defective satellite of hepatitis B virus). In some examples, the viral pathogen is HIV, HCV, EBV, HTLV-1, KSHV, or Ebola virus.

Examples of bacterial pathogens include, but are not limited to: Helicobacter pylori, Escherichia coli, Vibrio cholerae, Borelia burgdorferi, Legionella pneumophilia, Mycobacteria sps (such as. M. tuberculosis, M. avium, M. intracellular, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Bordetella pertussis, Shigella flexnerii, Shigella dysenteriae and Actinomyces israelii.

Examples of fungal pathogens include, but are not limited to: Cryptococcus neoformans, Histoplasma capsulatum, Coccidioides immitis, Blastomyces dermatitidis, Chlamydia trachomatis and Candida albicans.

Other pathogens (such as parasitic pathogens) include, but are not limited to: Plasmodium falciparum, Plasmodium vivax, Trypanosoma cruzi and Toxoplasma gondii. (Plasmodium species), amoebiasis (Entamoeba species), giardiasis (Giardia lamblia), toxoplasmosis (Toxoplasma gondii), cryptosporidiosis (Cryptosporidium species), trichomoniasis (Trichomonas vaginalis), Chagas disease (Trypanosoma cruzi), Leishmaniasis (Leishmania species), sleeping sickness (Trypanosoma brucei), amoebic dysentery (Entamoeba histolytica), acanthamoeba eeratitis (Acanthamoeba species), and primary amoebic meningoencephalitis (Naegleria fowleri)

Examples of helminth pathogens include Strongyloides stercoralis (causes strongyloidiasis); Onchocerca volvulus (causes river blindness/Robles disease); Loa (filarial nematode that causes Loa filariasis); and Wuchereria bancrofti (roundworm that causes lymphatic filariasis).

Antigens and antigenic epitopes associated with the various microbial and viral agents above are known. Moreover, antibody binding domains and scFv sequences targeting a vast number of biological targets are known in the art (see, e.g., WO2018/102795, which is incorporated herein by reference).

In one non-limiting example, sdAbs of the disclosure can be used to treat an HIV infection by binding to antigens associated with the Env protein from HIV. Similarly, antibodies developed against spike proteins of SARS-Cov2 can be used as a molecule from which recombinant binding domains can be obtained, cloned and used in an antigen recognition cassette of the disclosure. Such cassettes can then be used in the engineering of B cells for administering to a subject to allow for long term persistent response to SARS-Cov2 infection.

In another embodiment, antibody fragments (e.g., sdAbs, scFv etc.) and non-immunoglobulin binding domains made by a method the disclosure can be used to treat a subject with a cancer by binding to antigens associated with the cancer. Examples of cancer antigens can be found throughout herein. Examples of cancers that can be treated by sdAbs of the disclosure include, but are not limited to, non-Hodgkin's lymphoma, acute lymphoblastic leukemia, B-cell lymphoma, mantle cell lymphoma, multiple myeloma, acute myeloid leukemia, colorectal cancer, breast cancer, lung cancer, ovarian cancer, and renal cancer.

In another embodiment, sdAbs or other antibody fragments made by a method the disclosure can be used to treat a subject with an autoimmune disorder by binding to and preventing activation of cytokines or receptors associated with an autoimmune disorder, or prevent aggregations or plaques associated with an autoimmune disorder. Examples of autoimmune disorders that can be treated by the compositions and methods of the disclosure include, but are not limited to, Alzheimer's disease, Celiac disease, Addison disease, Graves disease, dermatomyositis, multiple sclerosis, rheumatoid arthritis, psoriasis, and inflammatory bowel disease.

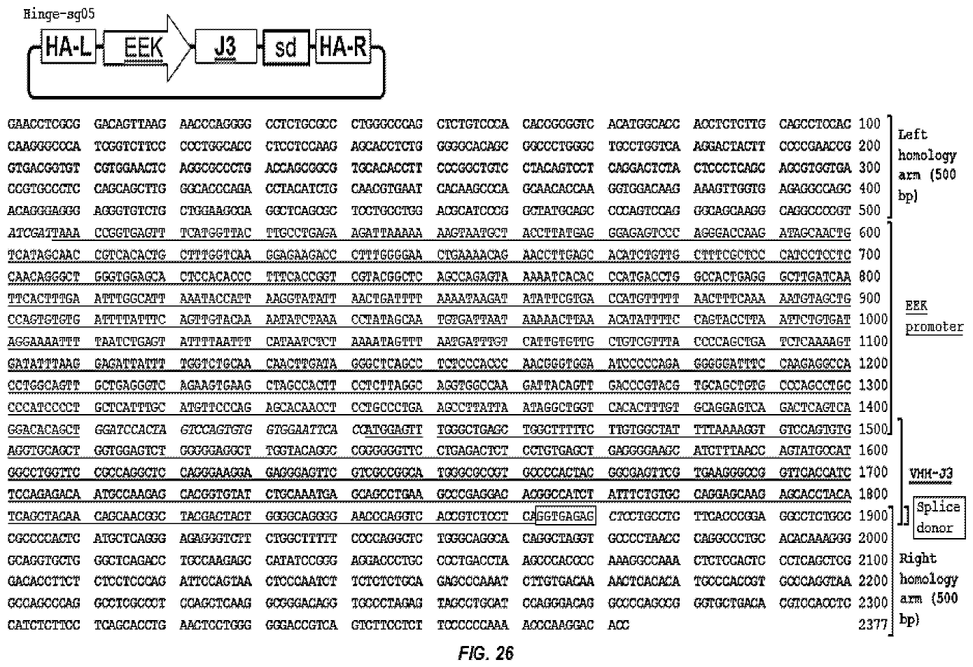

The disclosure provides an antigen recognition cassette comprising the general structure: --(promoter)-(binding domain)-(splice donor)--. The promoter can be any promoter that can function to elicit expression of an operably linked coding sequence. Various promoters are known in the art. As mentioned above, the promoter can be tissue specific, constitutive, or inducible. The binding domain comprises a nucleic acid sequence encoding a binding domain polypeptide. As described above, the binding domain polypeptide can be an antibody fragment, a receptor domain, an artificial polypeptide having affinity for a particular antigen or cognate. In some embodiments, the cassette can comprise the hinge and CH2 coding sequences of an Ig locus. The splice donor domain comprises a nucleic acid sequence that can interact with a splice acceptor domain. An exemplary antigen recognition cassette is provided in FIG. 25 (see also SEQ ID NO:12). As depicted in FIG. 25, the promoter is identified as beginning at basepair (bp) 1 to about 904. In one embodiment, the promoter sequence can be at least 80%, 90%, 95%, 98% or 99% identical to a sequence from 1-904 of SEQ ID NO:12 and which is capable of driving transcription of an operably linked coding sequence. As depicted in FIG. 25, the binding domain is identified as beginning at about bp 937 to about 1323 (with CDRs 1, 2, and 3 identified). In another embodiment, the binding domain can be at least 80%, 90%, 95%, 98% or 99% identical to a sequence from 937-1323 of SEQ ID NO:12. It should be noted that with respect to a particular binding domain and its specific affinity against a particular target the CDRs are typically more conserved and that any variation in sequence is more tolerable in areas outside the CDR sequences. As depicted in FIG. 25, the splice donor comprises bp 1457 to 1464 of SEQ ID NO:12. The antigen recognition cassette can also comprise additional components such as hinge domains and/or all or a portion of a constant heavy chain domain.

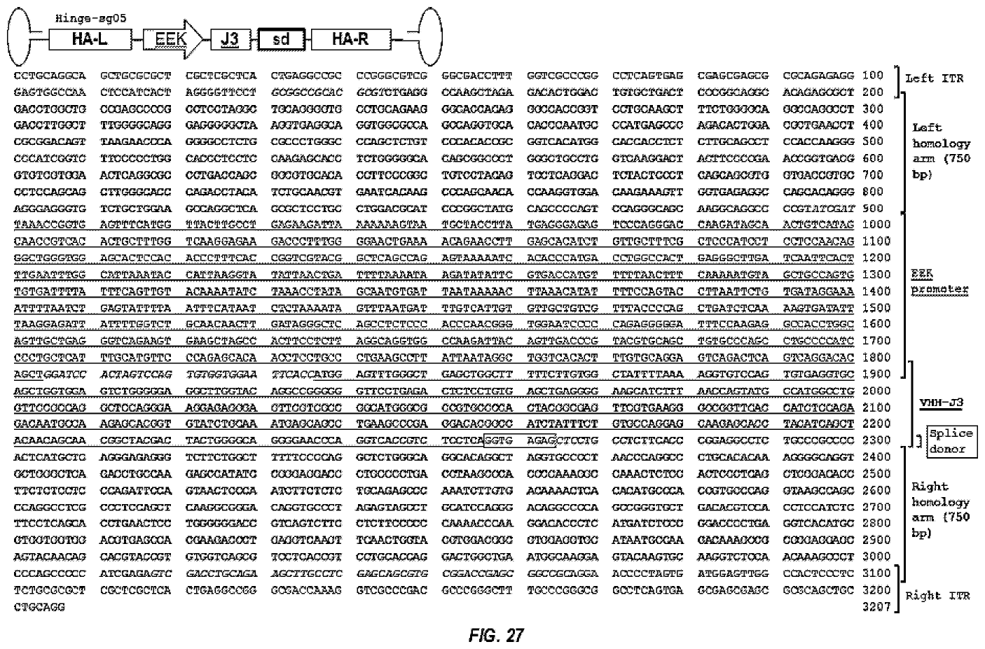

In some embodiments, the antigen recognition cassette is flanked by homology regions that have sequence homology to a site for insertion. In certain embodiments, the homology region has homology to an Ig region of a mammalian cell's genome. In some embodiments, that homology region is 25-750 bp long (e.g., 25, 50, 100, 200, 250, 300, 350, 400, 450, 500, 750 bp or longer). In some instances the homology region can be 500-1000 bp long. In certain embodiments, the homology region is 5′ to the promoter of the antigen recognition cassette and 3′ to the splice donor domain of the antigen recognition cassette. In other embodiments, the homology arms can be of different lengths. An exemplary construct is provided in FIG. 26 (see also SEQ ID NO:13).

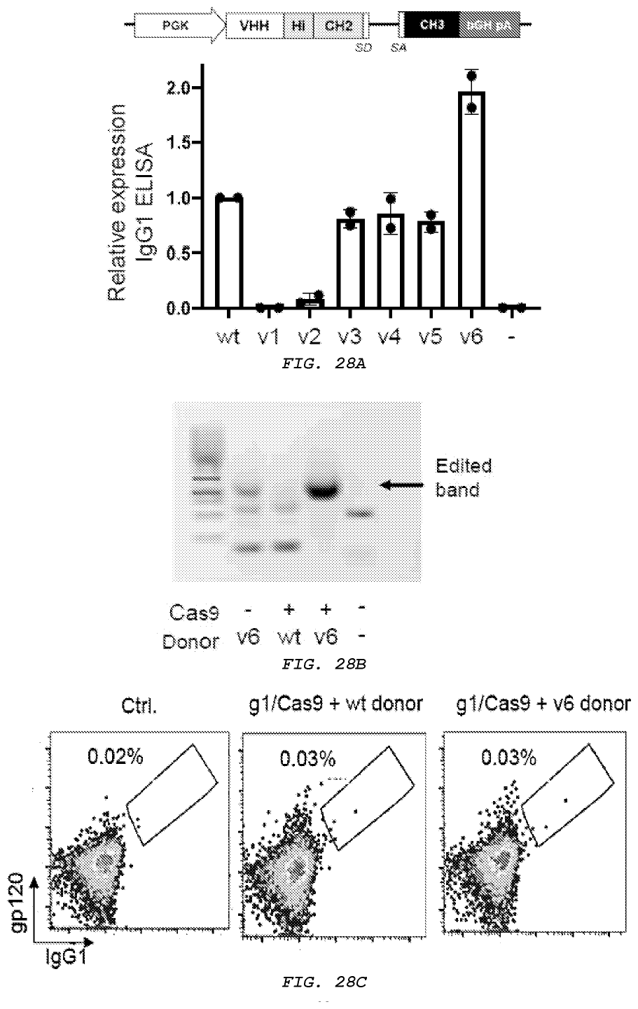

In still another embodiment, the disclosure provides a construct comprising an antigen recognition cassette with homology arms. In some embodiment, the construct is present in an AAV backbone. In one embodiment, the homology arms of a recognition cassette construct are flanked by ITRs of an AAV vector. An exemplary vector construct is provided in FIG. 27 (see also SEQ ID NO:14).

As will be readily apparent the polynucleotide constructs of the disclosure are modular in design comprising a promoter module, a binding domain module, a splice donor module, a homology module, and/or a vector module. One of skill in the art will readily recognize that the modules can be varied without undue experimentation. For example, the promoter module can be any number of different promoter types/sequences as are well known in the art. Moreover, the binding domain module can be any number of binding domain module sequences (see, e.g., WO2018/102795 at Table 5, listing VL, VH, VHH and other binding domains and CDRs and related sequences, which are incorporated herein by reference). The Homology module (Homology arms) can be any sequence that is designed to have homology to the site where the cassette is to be inserted. Typically, the homology arms will have homology to an Ig locus in a mammalian cell.

In one embodiment, the disclosure provides an ex vivo method of generating engineered B cells. The method comprises isolating B cells from a subject, contacting the isolated B cells with a vector comprising an antigen recognition cassette of the disclosure such that the antigen recognition cassette integrates into the B cell genome in an Ig locus, and culturing the cells. The cultured cells may be “banked” or stored for administration to a patient or subject to be treated. The patient of subject may be autologous with the cells or allogeneic. Methods of isolating B cells are known. For example, B-cells can be isolated by two main approaches: 1) Negative selection—in which B-cells remain “untouched” in their native state; this is advantageous as it is likely that B-cells remain functionally unaltered by this process or 2) Positive selection—in which B-cells are labelled and actively removed from the sample by FACS, MACS, RosetteSep or antibody panning. One or more isolation techniques may be utilized in order to provide an isolated B cell population with sufficient purity, viability and yield.

In another embodiment, the disclosure provides an ex vivo method of generating engineered precursor B cells. The method comprises isolating precursor B cells including, but not limited to, embryonic stem cells, hematopoietic cells or parenchymal cells that are induced to become stem cells, from a subject, contacting the isolated precursor B cells with a vector comprising an antigen recognition cassette of the disclosure such that the antigen recognition cassette integrates into the precursor B cell genome in an Ig locus, and culturing the cells. The cultured cells may be “banked” or stored for administration to a patient or subject to be treated. The patient of subject may be autologous with the cells or allogeneic.

The following examples are intended to illustrate but not limit the disclosure. While they are typical of those that might be used, other procedures known to those skilled in the art may alternatively be used.

### EXAMPLES

HIV-specific bn-sdAbs neutralize HIV. Camelid VHH domains previously reported to have broadly neutralizing activity against HIV (described in Table 1) were fused to the Hinge-CH2-CH3 domains of human IgG1 to create sdAbs.

TABLE 1
Summary of previously described VHH
domains and sdAbs with anti-HIV activity

| | | | Published | | |
|---|---|---|---|---|---|
| | | % neutralization | median | | Epitope on |
| sdAb | | of HIV strains | IC50 | | HIV Env |
| VHH | Origin | tested | (μg/mL) | Format | protein |
| 9 | Dromedary | 48% (10/21) | 0.18 | VHH-Fc | CD4bs/CD4i |
| 28 | Dromedary | 62% (13/21) | 0.277 | VHH-Fc | CD4bs/CD4i |
| A6 | Dromedary | 76% (16/21) | 0.224 | VHH-Fc | CD4bs/ |
| | | | | | CD4i/V2 |
| J3 | Llama | 98% (57/58) | 0.93 | VHH | CD4bs |
| A14 | Llama | 74% (45/61) | 0.53 | VHH | CD4bs |
| B9 | Llama | 77% (47/61) | 0.85 | VHH | CD4bs |
| 3E3 | Llama | 76% (58/71) | 0.82 | VHH | CD4bs |

CD4bs - CD4 binding site; CD4i - CD4-induced epitopes; V2 - the V2 apex

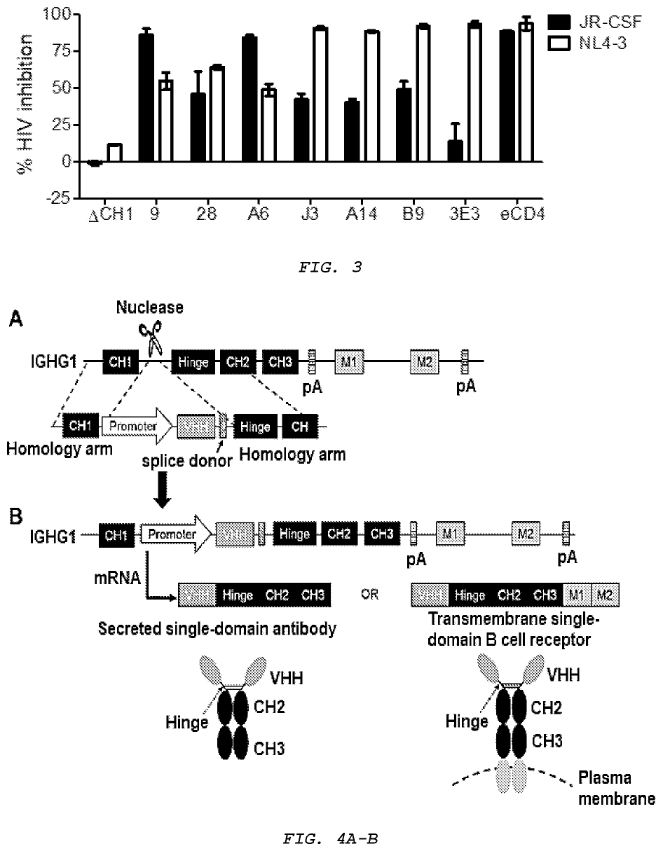

The antibodies were produced in 293T cells by calcium phosphate transfection of plasmids containing the sdAb sequences downstream of a CMV promoter, and the presence of HIV binding antibodies secreted into the culture supernatants was confirmed using an ELISA for binding to the HIV Env gp120 subunit. Antibody-containing supernatants were incubated with 2 different strains of HIV (R5-tropic JR-CSF and X4-tropic NL4-3), and HIV neutralization capabilities were determined using the GHOST cell assay as described in Cecilia et al., (Neutralization profiles of primary human immunodeficiency virus type 1 isolates in the context of coreceptor usage. J Virol 72: 6988-6996 (1998)). All sdAbs were inhibitory against both strains of HIV tested (see FIG. 3), though some were more effective against JR-CSF (9, 28, A6) whereas other were more effective against NL4-3 (J3, A14, B9, and 3E3). The ACH1 supernatant is a negative control generated from cells transfected with plasmids expressing just the Hinge-CH2-CH3 domains of human IgG1 and lacking an anti-HIV VHH domain. eCD4-Ig (eCD4) was included as a positive control secreted protein known to neutralize many strains of HIV.

Activity of spCas9 gRNAs at on- and off-target IgG genes. The activity of 10 spCas9 gRNAs (described in Table 2) and 4 Cpf1 gRNAs (described in Table 3) targeting an intron between the hinge and CH2 exons of IgG1 were assessed at the on-target IGHG1 gene, 4 major off-target regions (IGHG2, IGHG3, IGHG4, and IGHGP). As well, spCas9 gRNAs targeting the IGHG1 intron preceding the CH3 exon were assessed for on- and off-target activity (Table 4). These off-target loci comprise 3 genes and a pseudogene that are all >96% homologous to IGHG1 and thus have a high possibility of off-target activity.

TABLE 2
Summary of on and off-target cutting efficiency
of tested spCas9 guide RNAs targeting the
human IGHG1 intron preceding the Hinge exon.

| | | | Off- |
|---|---|---|---|
| | Genomic | Cutting | target |
| | sequence | effi- | (IGG) |
| | targeted | ciency, | cutting |
| Guide | by gRNA (5′-3′)* | ICE | detected |
| identity | (SEQ ID NO:) | (%) | by ICE |
| sg01 | AGGCTAGGTGCCCCTAACCC (15) | 70 | +/− |
| sg02 | TAGCCGGGATGCGTCCAGGC (16) | 40 | + |
| sg03 | TGCATAGCCGGGATGCGTCC (17) | 86 | +++ |
| sg04 | CTCCGGGTGAAGAGGCAGAC (18) | 47 | − |
| sg05 | TCCGGGTGAAGAGGCAGACG (19) | 51 | − |
| sg06 | ACCCAGGCCCTGCACACAAA (20) | 72 | − |
| sg12 | GATTGGGAGTTACTGGAATC (21) | 46 | +/− |
| sg16 | GCAGAGGCCTCCGGGTGAAG (22) | 20 | − |
| sg17 | GCCCCGTCTGCCTCTTCACC (23) | 32 | − |
| sgCOR2 | CCGTCTGCCTCTTCACCCGG (24) | 93 | + |

IGG: IGHG2, IGHG3, IGHG4, or IGHGP
*Shown are: the genomic sequences. As an example and for clarification, the sg01 gRNA would include the RNA sequence 5′AGGCUAGGUGCCCCUAACCC 3′

TABLE 3
Summary of on and off-target cutting efficiency
tested Cpf1 guide RNAs targeting the human
IGHG1 intron preceding the Hinge exon.

| | | | Off- |
|---|---|---|---|
| | | | target |
| | Genomic | | (IGG) |
| | sequence by | Cutting | cutting, |
| | targeted | effi- | measur- |
| Guide | gRNA (5′-3′) | ciency, | able |
| identity | (SEQ ID NO:) | ICE (%) | by ICE |
| Cpf1-gl | TCCCCAGGCTCTGGGCAGGCA (25) | 0 | NA |
| Cpf1-g2 | CCCCAGGCTCTGGGCAGGCAC (26) | 0 | NA |
| Cpf1-g3 | CCCAGGCTCTGGGCAGGCACA (27) | 70 | — |
| Cpf1-g4 | TGTGCAGGGCCTGGGTTAGGG (28) | 2 | NA |

TABLE 4
Summary of on and off-target indel generation
for indicated spCas9 guide RNAs targeting
the human IGHG1 intron preceding the
CH3 exon.

| | | | Off-target |
|---|---|---|---|
| | Sequence targeted | indel | (IGG) |
| | by gRNA | effi- | indels |
| Guide | gRNA (5′-3′) | ciency, | detected |
| identity | (SEQ ID NO:) | ICE (%) | by ICE |
| CH3-g1 | ATGTGGCCCTCGCACCCCAC (29) | 41 | − |
| CH3-g2 | AAGCCAAAGGTGGGACCCGT (30) | 23 | − |
| CH3-g3 | AGCCAAAGGTGGGACCCGTG (31) | 56 | +/− |
| CH3-g4 | GTGGGACCCGTGGGGTGCGA (32) | 0 | − |
| CH3-g5 | CATGTGGCCCTCGCACCCCA (33) | 70 | + |

Briefly, gRNAs were synthesized in vitro, complexed with recombinant spCas9 protein, and nucleofected into K562 cells. After 5 days, genomic DNA was isolated, and PCR and Sanger sequencing analyses were performed for all 5 loci. The presence of DNA double-stranded breaks (DSBs) was inferred by observing indels, which were quantified by ICE as described in Hsiau et al., (Inference of CRISPR Edits from Sanger Trace Data. bioRxiv: 251082 (2019)). On-target DSBs were generated by all guides, though the total detectable activity varied. Three spCas9 guides (sg02, sg03, and sgCOR2) exhibited significant off-target activity at one or more of the homologous IgG genes, implying lack of suitability for this application, whereas the other 7 targeting the same intron showed little to no off-target cutting as detected by this assay (limit of detection ˜2%) (see FIG. 5).

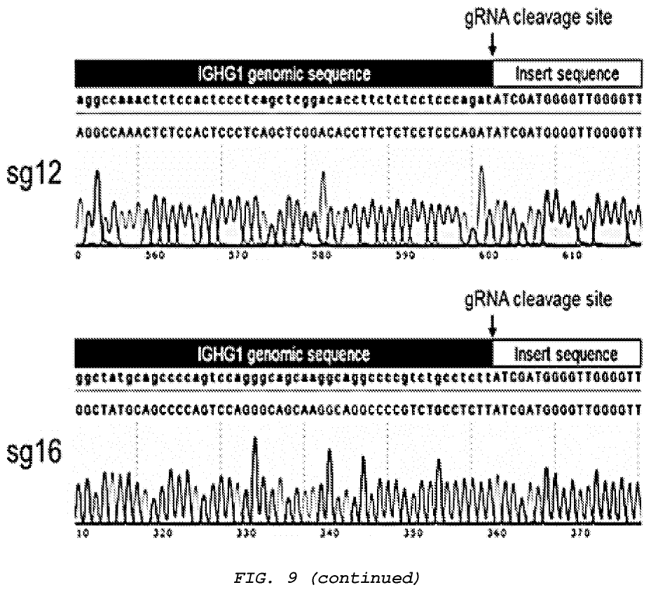

Genome editing at the IGHG1 locus using spCas9 RNPs and matched plasmid homology donors. K562 cells were nucleofected with spCas9 RNPs containing the indicated guide RNAs, in combination with a series of matched plasmid homology donors with different lengths of homology arms, as indicated. A unique series of homology donors were paired with each guide, since the exact DNA break site and thus preferred location for gene insertion is different for each gRNA. After 3 weeks, stable GFP expression, indicating site-specific genome editing, was measured by flow cytometry. The gRNA used was the most important source of variation in the final GFP levels; all homology arm designs for sg05 were superior to other gRNA/homology donor pairs (see FIG. 7).

Genome editing at the IGHG1 locus using spCas9 and AAV6 homology donors. Homology donor cassettes were packaged into AAV6 vectors using standard methods to produce AAV vectors (triple transfection and iodixanol gradient centrifugation) (see FIG. 8A). K562 cells were transduced with AAV6 vectors, and then nucleofected with matched spCas9 RNPs. Stable GFP expression was measured after 3 weeks by flow cytometry. Similar to the experiments described above, using the plasmid homology donors, guide sg05 produced the highest rates of gene insertion (See FIG. 8B). The expected outcome of genome editing using these homology donors is shown in FIG. 8C, including a schematic of the design of the ‘in-out PCR’ assay used to confirm site-specific gene insertion. One primer is located in the genome outside of the homology arms, and another primer is found within the GFP insertion cassette. A band will be produced only if site-specific gene insertion has occurred. In-out PCR provided for site-specific gene insertion in cells that received AAV6 homology donors and spCas9, but not in cells receiving AAV6 only, confirming that the amplified DNA results from site-specific gene insertion (see FIG. 8D).

Genome editing at the IGHG1 locus produces HIV-specific bn-sdAbs in Raji cells and Ramos cells. Raji cells and Ramos cells (human B cell lines) were nucleofected with RNPs comprising spCas9 and gRNA sg05, together with matched plasmid homology donors, designed to insert expression cassettes for either PGK-GFP-pA, or the bn-sdAbs A6 or J3 (Table 1) plus a splice donor (sd). Expression of bn-sdAbs is driven by the B cell-specific EEK promoter. A 10-fold increase in stable GFP expression after 2 weeks was observed in Raji cells receiving donor plasmids plus sg05 RNPs compared to donor plasmid only, consistent with site-specific gene insertion stimulated by the targeted DSB (see FIG. 10B). Raji cells which were edited with the two sdAb homology donors were stained for cell surface human IgG1 and binding by HIV Env gp120 (see FIG. 10C). Cells receiving Cas9 RNPs plus plasmid donor exhibited double-positive cells (gated) staining for both IgG1 and HIV gp120. The frequency of this edited population was similar to that observed with the GFP donor cassette. In contrast, no clear population of double-positive cells was observed in untreated cells, or in cells receiving plasmid donors only. Together, these results support that double-positive cells were observed as a result of site-specific genome editing with the bn-sdAb homology donors. Moreover, the direct correlation between the fluorescence signal in both channels supports that the same surface protein is responsible for binding both the anti-IgG antibody and the HIV Env gp120 protein.

Increased stable GFP expression after 2 weeks was observed in Ramos cells receiving the GFP plasmid donor and Cas9 RNPs, consistent with site-specific gene insertion (see FIG. 11A). Ramos cells edited with sdAb homology donors were stained for cell surface human IgG1 and HIV env gp120. Ramos cells receiving Cas9 RNPs plus donor plasmid exhibited double-positive cells staining for both IgG and HIV gp120 (see FIG. 11B). The frequency of this edited population was similar to that observed with the GFP donor cassette. In contrast, no clear population of double-positive cells was observed in untreated cells or in cells receiving plasmid only. Together, these results support that double-positive cells were observed as a result of site-specific genome editing by the bn-sdAb homology donors. Moreover, the direct correlation between the fluorescence signal in both channels supports that the same surface protein is responsible for binding both the anti-IgG antibody and the HIV Env gp120 protein.

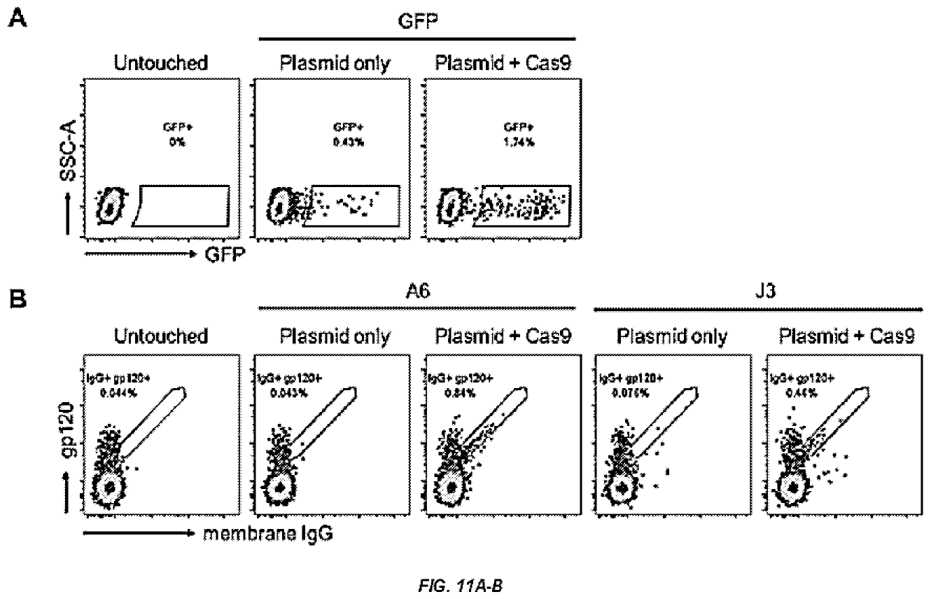

After enrichment, bn-sdAb but not GFP edited cells secrete human IgG. Engineered Raji and Ramos cells were FACS sorted for surface human IgG1 (A6 and J3 edited cells) or for GFP (GFP edited cells) and expanded in culture (see FIG. 13). The frequency of HIV-specific cells was measured by flow cytometry. A significant increase in the frequency of double-positive (IgG⁺ gp120⁺) cells compared to the pre-sort frequency (˜0.5-1.2% in FIGS. 9-10) was observed, suggesting effective enrichment for the sdAb genome edited cells (see FIG. 13A). The concentration of human IgG in the supernatant of engineered, enriched Raji and Ramos cells was quantified by ELISA. Secreted antibodies were detected from both cell lines following engineering with the bn-sdAbs A6 or J3, but not from GFP-edited cells (see FIG. 13B). The results suggest that these antibodies are produced as a consequence of site-specific genome editing as illustrated in FIG. 2.

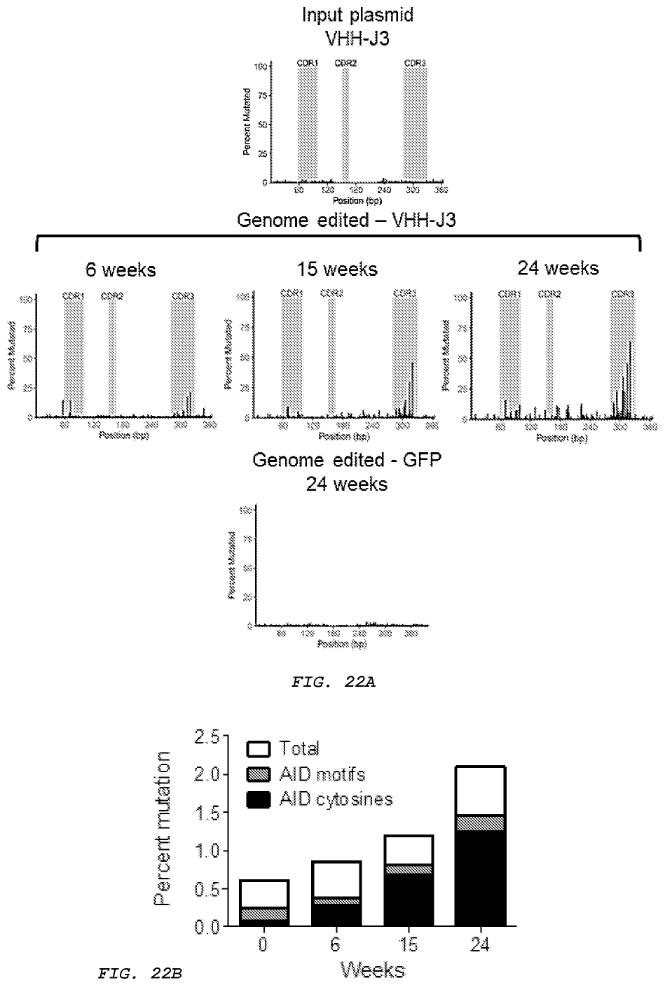

Using similar methodology, additional antigen recognition cassettes were inserted at the CH1-Hinge intron. The IGHG1 locus was edited by inserting various alternate protein domains that bind to HIV gp120. PGT121 is a human anti-HIV bnAb and scFv cassettes were generated in both the heavy chain-light chain (HL) and light chain-heavy chain (LH) orientations using standard (G₄S)₃ linkers. CD4-mD1.22 is an engineered variant of domain 1 of CD4 that can bind to and neutralize HIV, but does not bind to MHC class II molecules. Raji cells were genome edited using spCas9 RNPs comprising sg05 (Table 2), and corresponding plasmid homology donors, by nucleofection. Cell surface expression of the expected resulting single-chain constructs was detected by flow cytometry to detect IgG expression and binding to recombinant gp120 (FIG. 18A). In addition, a tandem bispecific sdAb was generated by inserting a tandem cassette of VHH-A6 and VHH-J3 joined by a (G₄S)₃ linker (FIG. 18B).

Anti-HIV activity of antibodies produced by engineered B cell lines. Supernatants from engineered, enriched Raji and Ramos cells were diluted and mixed with 2 different strains of HIV (R5-tropic JR-CSF and X4-tropic NL4-3), and HIV neutralization capability was assayed using the GHOST cell assay. Supernatants from transiently transfected 293T cells receiving expression plasmids for the same bn-sdAbs were included as a positive control. FIG. 14A presents the effect of supernatants on HIV infection for A6-containing supernatants. FIG. 14B presents the effect of supernatants on HIV infection for J3-containing supernatants. Inhibition of both strains of HIV was observed with all 3 supernatants, confirming that the sdAb antibodies produced by the genome engineered B cells possess anti-HIV activity. Note that the relative efficiency of each antibody against the two different viruses was conserved across antibody sources. That is, A6 antibodies harvested from the supernatants of transfected 293T cells or edited Raji and Ramos cells was similarly effective against both JR-CSF and NL4-3 viruses, whereas J3 antibodies were more effective against NL4-3 than JR-CSF in all supernatants tested.

Quantification of anti-HIV activity of bn-sdAbs produced by transfection and genome editing. TZM-bl cells were used to assay the activity of A6- and J3-containing supernatants from transfected 293T cells or gene edited Raji or Ramos cells against 2 strains of HIV (JR-CSF or NL4-3). The relative efficiency of each antibody against either strain of HIV was conserved regardless of whether it was produced in 293T cells by transfection or from genome edited B cells (see FIG. 16). Consistent with the results in FIG. 14 using a different reporter cell line (GHOST cells) to measure HIV infection, the anti-HIV activity of A6 was somewhat similar against both strains of HIV, whereas J3 was significantly more effective against NL4-3 than JR-CSF. IC-50 values were also calculated and are reported in Table 5. These were highly similar for each antibody across the 3 cell sources, demonstrating that bn-sdAbs produced by genome editing have similar specific activity to recombinant antibodies generated by transfection of 293T cells.

TABLE 5
Antibody neutralization efficiency from
supernatants of 293T cells transiently transfected with an sdAb
expression cassette and engineered human B cell lines.

| | A6, IC50 | (ng/mL) | J3, IC50 | (ng/mL) |
|---|---|---|---|---|
| Cell type/treatment | JR-CSF | NL4-3 | JR-CSF | NL4-3 |
| 293T/transfection | 436.8 | 45.3 | 8274 | 113.5 |
| Raji/genome editing | 265.7 | 67.8 | >8000 | 367.9 |
| Ramos/genome editing | 487.7 | 63.7 | >7800 | 125.0 |

Genome editing and in vitro differentiation of primary human B cells. Genome editing was performed at the CCR5 locus in primary human B cells using site-specific zinc finger nucleases (ZFN) or spCas9/gRNA targeting the CCR5 locus, combined with matched AAV6 CCR5-GFP homology donors (see FIG. 16). The B cell activation and differentiation protocol was adapted from Jourdan et al., (An in vitro model of differentiation of memory B cells into plasmablasts and plasma cells including detailed phenotypic and molecular characterization. Blood 114: 5173-5181 (2009)). Briefly, B cells were activated for 2 days, then transduced with AAV6 vectors packaging CCR5-GFP homology donor genomes and electroporated with in vitro transcribed CCR5 ZFN mRNA or CCR5 gRNA/Cas9 RNPs. After 2 more days of activation, a different mix of cytokines are applied to cause cells to adopt a plasmablast phenotype, followed by a third mix on day 7. Ten days after cell isolation/thawing, cells were assessed for site-specific genome editing by flow cytometry. As shown in FIG. 16B, stable GFP expression in primary human B cells was observed after genome editing with CCR5-specific ZFN mRNA and AAV6-GFP homology donors, at several different AAV6 doses (MOIs). Secretion of human antibodies was assessed by genome edited primary human B cells after 10 days of differentiation, by specific ELISA. Both IgM and IgG were detected, suggesting that cells had been successfully differentiated towards an antibody-secreting cell phenotype (see FIG. 16C). Primary human B cells were electroporated with spCas9/gRNA RNPs targeting the CCR5 locus and transduced with a matched AAV6 homology donor at a MOI of 105 encoding GFP. Stable GFP expression was measured after 8 days by flow cytometry (see FIG. 16D). Together, these results demonstrate methods to perform genome editing using primary human B cells and, through in vitro treatments, to measure their ability to secrete antibodies following genome editing, and during differentiation.

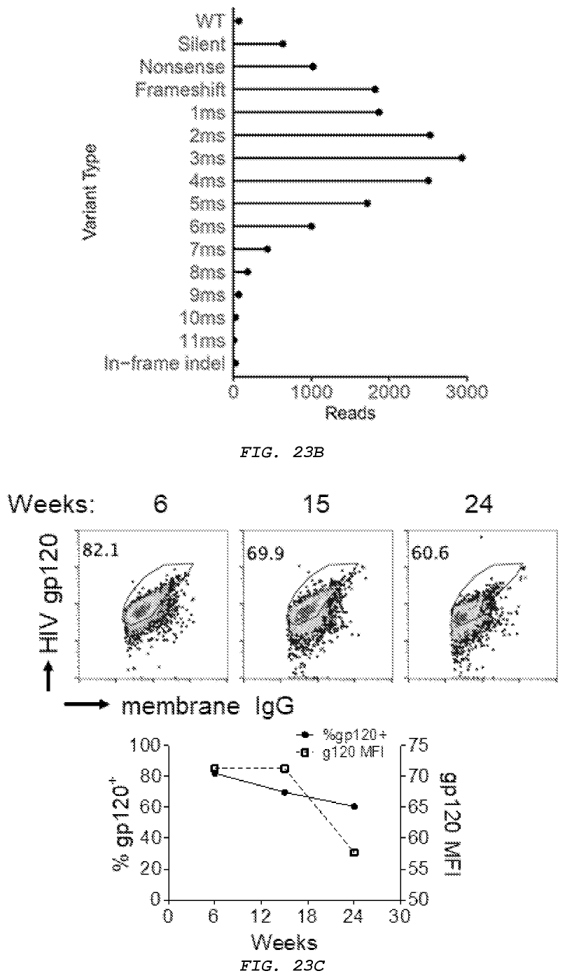

Different insertion sites can produce antibody-like molecules using the methods and compositions of the disclosure. FIG. 20 presents a schematic of genome editing at the IGHG1 locus by targeting the intron upstream of CH3. As an example, the use of a VHH domain is shown to create an sdAb, although other antibody or protein domains (e.g., other binding domains and related sequence), including those described in FIG. 2, could be used in place of the VHH domain. Homology-directed repair (HDR), catalyzed by site-specific DNA double-stranded breaks produced by a targeted nuclease such as spCas9/gRNA promotes insertion of the indicated homology donor cassette in the intron upstream of CH3. In this example, the hinge and CH2 exons of the constant region are included in the inserted cassette, which comprises a promoter (in these examples a B cell-specific EEK promoter), a functional domain (for example a VHH domain), the hinge and CH2 exons and a splice donor, and is flanked by sequences with homology to the Ig locus (homology arms). In this design, the hinge and CH2 sequence can be modified, for example, by codon wobbling to reduce homology to the endogenous hinge and CH2 sequences and the CH2 sequence can be further modified to include mutations that enhance antibody-dependent cell-mediated cytotoxicity (ADCC), antibody-dependent cellular phagocytosis (ADCP), complement activation, or half-life in circulation. For example, the CH2 sequence can be designed to include the ‘GASDALIE mutations’ (G236A, S239D, A330L, I332E), which have been reported to enhance ADCC (Smith et al., Proc Natl Acad Sci USA 109:6181-6186, 2012). Alternatively, the CH2 domain could be replaced with a linked anti-CD16 nanobody or scFv as another mechanism to create a single-chain molecule that triggers ADCC. Finally, the Hinge and CH2 domains could be omitted, to generate a minibody containing only the VHH (or other functional domain) fused to CH3, which is still capable of dimerization and can access some epitopes due to its smaller size. Following HDR, the VHH cassette, hinge and modified CH2 sequences are inserted between the CH2 and CH3 exons of IgG1 in the human genome, as indicated in FIG. 20B. The inserted promoter drives transcription, and the splice donor after the inserted CH2 exon splices the resulting RNA transcript with the downstream genomic CH3 exon to produce the indicated single-chain antibody. Exclusion of the membrane exons M1 and M2 results in production of the secreted Ab, while their inclusion results instead in the transmembrane BCR.

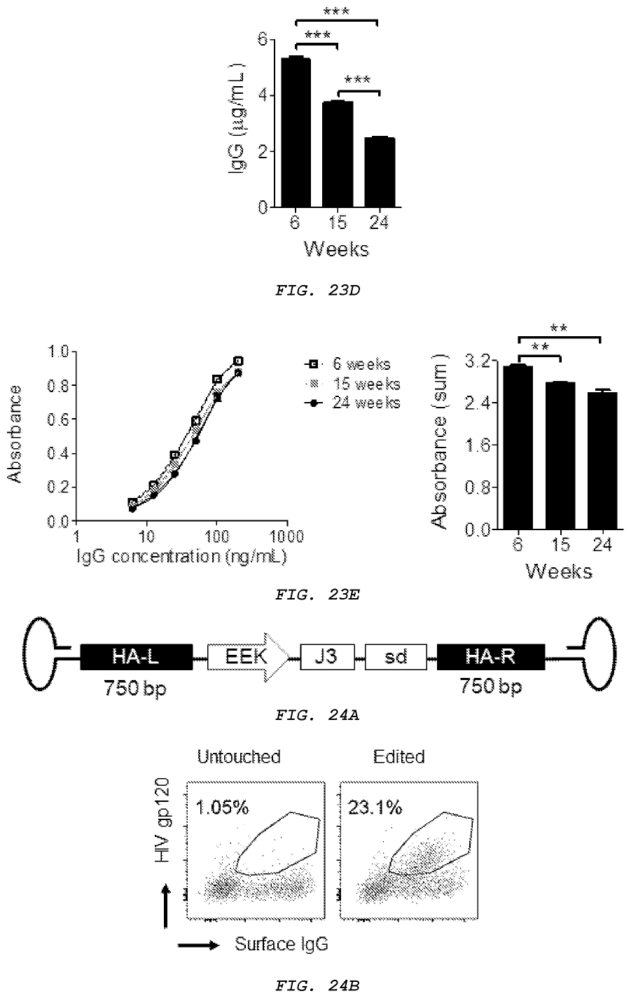

Also demonstrated is editing at the intron upstream of CH3 in IGHG1 using spCas9 complexed with guide RNAs (gRNAs) (FIG. 21). The activity of 5 spCas9 gRNAs (described in Table 4) targeting the intron upstream of the CH3 exon of IgG1 were assessed at the on-target IGHG1 gene site, as well as at 4 major predicted off-target regions (IGHG2, IGHG3, IGHG4, and IGHGP). Activity was measured by indel generation, which is one result after repair of DSBs, by Sanger sequencing (Hsiau et al. bioRxiv 2019 DOI: 10.1101/251082). On-target indels were observed for 4/5 guides. Moderate off-target activity was observed for CH3-g5 at IGHG4, and minor activity at IGHG2 for g3 was detected (limit of detection ˜2%). Homology-directed repair (HDR) was measured using Sanger sequencing for all 5 gRNAs (Table 4), following co-nucleofection of Cas9 RNPs and matched ssODN homology donors containing 40 bp homology arms on either side of the predicted Cas9 break site, to insert an XhoI restriction site. All 5 guides were able to support HDR (including CH3-g4 that did not exhibit detectable on-target indel formation), and CH3-g1 supported the highest HDR levels.

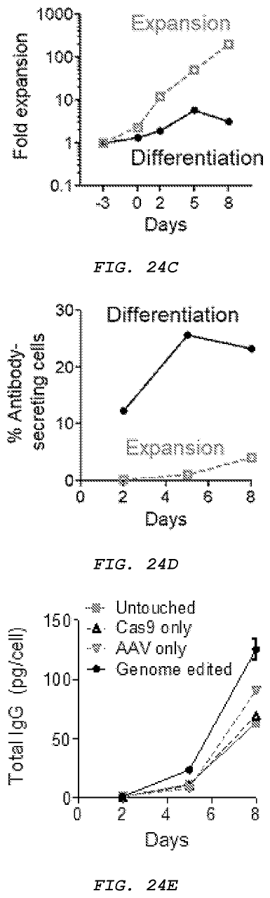

Moreover, the data shows evidence of somatic hypermutation. FIG. 22 shows somatic hypermutation occurring in a VHH-J3 sequence inserted at the IGHG1 locus by genome editing over time in Raji cells. VHH-J3 sequences specifically inserted at the IGHG1 locus were amplified by in-out PCR, and a nested PCR strategy was used to add partial Illumina adapters. The input plasmid was directly amplified using the internal primer pair. Following Illumina next-generation sequencing (NGS), the mutagenesis frequency (% of reads at each position that are not the original nucleotide) was quantified over the length of the sequence. Compared to the minimal mutagenesis in the input plasmid, increasing mutations were observed over time, particularly in CDR3, in the VHH-J3 sequence in the genome-edited Raji cells. In contrast, after 24 weeks, minimal mutagenesis was observed in the first 400 bp of a GFP sequence inserted into the same site in IGHG1 in Raji cells. The observations that total mutagenesis increased over time and with enrichment of the frequency of mutations within AID hotspots is consistent with ongoing somatic hypermutation. Within the motifs, mutagenesis was strongly localized at cytosines within the hotspot motifs, as would be expected for AID mutagenesis.

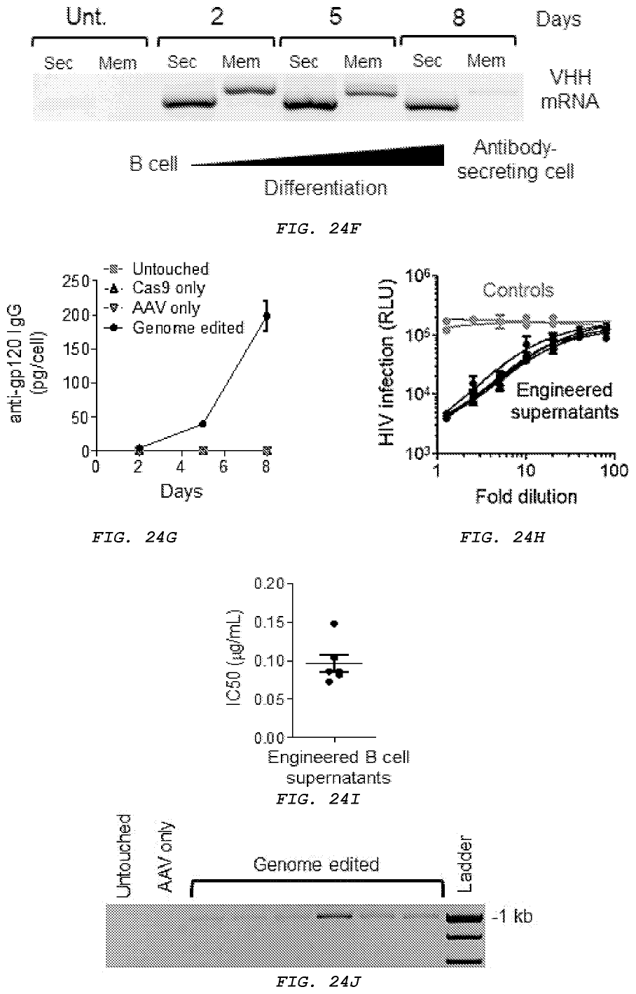

Further, FIG. 23 demonstrates that somatic hypermutation can alter the coding sequence of the gene. Protein sequences of NGS reads were classified based on the DNA sequence alterations observed after 24 weeks (ms: missense, reflecting the number of amino acid substitutions in the sequence). The majority of sequences at this point are expected to harbor changes to the CDR3 protein sequence. Surface VHH-J3 expression in edited Raji cells was characterized over time by flow cytometry, showing that both the frequency of J3-expressing cells as well as the intensity of gp120 staining (MFI: median fluorescence intensity; a surrogate for affinity for HIV antigen) decreased over time. Note that the cells were cultured in the absence of any selection pressure to maintain or improve gp120 binding. Total IgG secretion was quantified by ELISA from 500,000 engineered Raji cells after 2 days. The decline in total antibody secretion from an equal number of cells may reflect the impact of nonsense/frameshift mutations ablating protein translation in some cells, as observed by surface staining. The avidity of secreted VHH-J3 was quantified over time by gp120 ELISA. A dilution series containing normalized amounts of total IgG (quantified by ELISA) from each time point was used to measure absorbance at each point. The total absorbance sum was quantified showing a significant decline in absorbance even at equal amounts of antibody. This suggests that, even among secreted antibody, somatic hypermutation caused a decline in the avidity of the antibody population and was functionally altering the antibodies. In an in vivo setting of a germinal center reaction, such somatic hypermutation would instead be expected to lead to affinity maturation rather than the decline in function that was observed in vitro as a result of entropic mutagenesis in the absence of selective pressure.

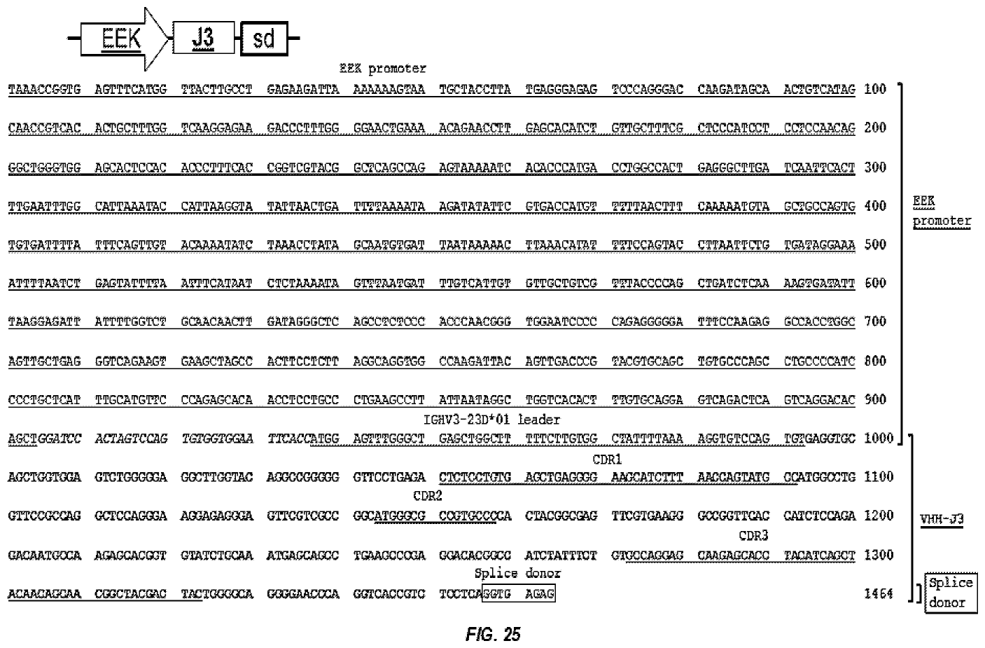

Experiments were also performed to look at in vitro differentiation, and secretion of functional anti-HIV antibodies from primary human B cells engineered by insertion of the EEK/VHH-J3/splice donor cassette upstream of the hinge exon of IGHG1. B cells were transduced with AAV6 homology donors followed by electroporation with spCas9 RNPs containing sg05 (Table 2). Surface expression of VHH-J3 sdAb in untouched and genome edited cells after 8 days was measured by flow cytometry (FIG. 24B). In addition, primary B cells were subject to two different cell culture protocols: an expansion protocol using ImmunoCult™-ACF Human B Cell Expansion Supplement (Stem Cell Technologies) and a differentiation protocol adapted from Jourdan et al. (Blood 114: 5173-5181, 2009). The expansion protocol yielded robust (>200-fold) expansion over 11 days of culture, whereas minimal expansion was observed with the differentiation protocol (FIG. 24C). The differentiation protocol converted a significant portion of B cells into an antibody-secreting cell phenotype (CD20-CD27+CD38hi) relative to the expansion protocol. ELISA was used to measure secretion of total IgG in the supernatant of cells treated with the indicated editing reagents and subject to the differentiation protocol. IgG concentrations were normalized by the number of viable cells and IgG secretion per cell increased over time in all populations, consistent with differentiation towards antibody-secreting phenotype. RT-PCR of RNA from untouched or engineered cells at indicated days post-editing shows specific expression of VHH-J3 mRNA in engineered cells. While initially both the membrane and secreted splice isoforms are detected, as the cells are differentiated over time the membrane isoform is lost while the secreted form continues to be detected. This suggests that splicing of the chimeric antibody transgene is being regulated by the differentiation of the B cell, in the same way as occurs for an endogenous antibody. HIV-specific human IgG detected by ELISA was present in the supernatant from cells genome edited with both spCas9/gRNA and AAV6 homology donors (“genome edited”), with expression levels per cell tracking with the total IgG secretion per cell measured in panel (FIGS. 24D and G). A concentration-dependent neutralization of HIV infection was achieved using supernatants from genome edited cells (engineered supernatants), whereas no anti-HIV activity was present in supernatants from untouched cells or cells that received AAV6 only (controls). IC₅₀ values for HIV inhibition in supernatants from engineered B cells were calculated from HIV inhibition results. The measured IC₅₀ closely matches that previously determined for VHH-J3 produced by transient transfection of 293T cells. Site-specific insertion of the VHH-J3 cassette was confirmed by in-out PCR to be only detected in the genomic DNA from cells that received both AAV6 and spCas9/gRNA.

Editing at an alternate location in IgG1, in the intron between CH2 and CH3. This approach is described above and with reference to FIG. 20, with data describing the gRNAs one would use to achieve such editing shown in FIG. 21. This editing approach has the advantage that it allows insertion of more than just an antigen-binding domain; specifically, the inserted sequence also comprises the Hinge and CH2 domains of the antibody. This thereby allows for the customization of additional part(s) of the constant region of the antibody. This is useful in order to incorporate mutations into CH2 that can enhance properties of the antibody.

The disclosure describes how the homology donor cassette is optimized to achieve the desired edits.

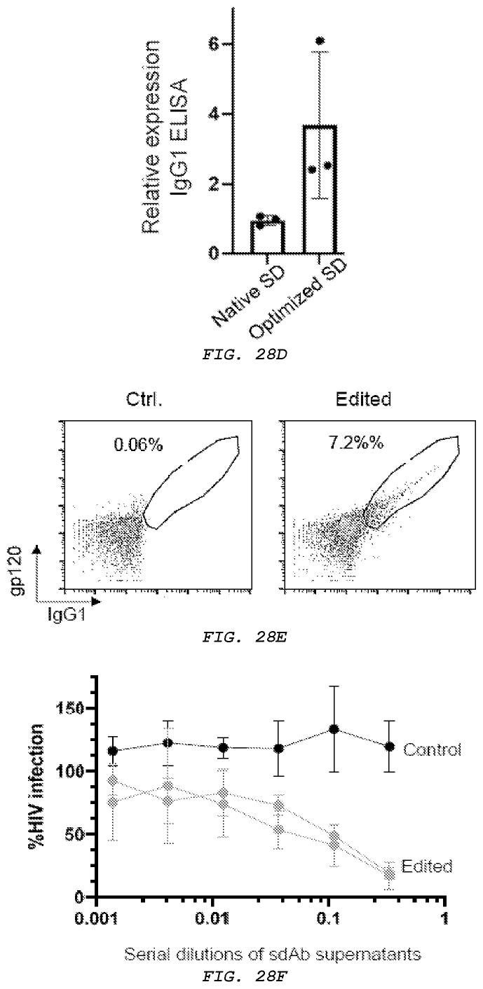

Specifically, as already disclosed above, it is useful to reduce homology between the Hinge-CH2 sequences in the cassette to be inserted and the endogenous Hinge and CH2 domain sequences. Retaining such homology was hypothesized to present alternate or competing stretches of sequence homology between the donor and the endogenous Ig locus, beyond the ‘homology arms’ in the constructs that are designed to direct the desired insertion events. To do this, six different codon wobbled sequences were constructed. Design ‘v6’ was chosen as it also resulted in the highest levels of antibody expression when examined as the final anticipated sequence of the recombinant protein (FIG. 28A). Gene insertion was detected after editing with the ‘v6’ but not wild-type donor, confirming the importance of codon wobbling for this approach (FIG. 28B).

The splice donor was also optimized to support expression of an sdAb after editing, since the original sequence did not support expression after site-specific insertion (FIG. 28C-D). The new SD sequence is shown in FIG. 20D. Successful gene expression, and the production of functional anti-HIV sdAbs after editing, is shown in FIG. 28E-F.

In FIG. 19, this editing approach could be done at additional locations. Specifically, FIG. 19A shows that editing could be achieved at the constant regions of immunoglobulin genes other than IgG1, including for example, IgG4.

FIG. 19B also shows that editing can also be achieved at different locations within the constant region of a chosen Ig gene, including towards the end of the antibody exons. In this way, one could thereby replace the entire Fc region of the antibody. This would enable substitution of the endogenous sequences with a customized antibody sequence, for example, with alternate CH3 sequences or mutations in CH3 that could enhance the half-life of the antibody. It also supports the replacement of the entire constant region sequences with non-antibody sequences.

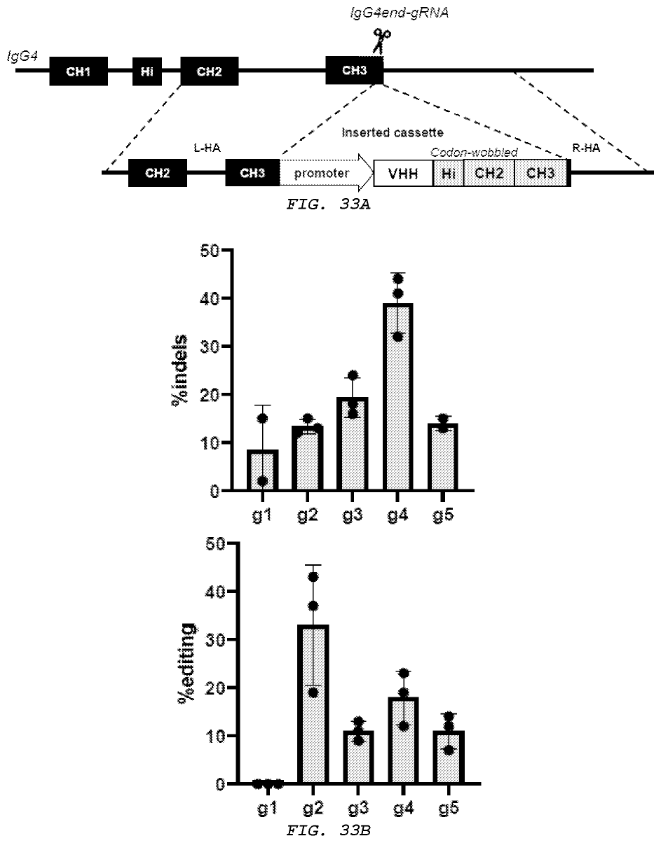

FIG. 33 shows an editing approach, including the design of the homology donor and gRNAs, to allow insertion of a complete sdAb sequence at IgG4.

Table 6 shows the sequence of the IgG4-end series of gRNAs that can be used.

TABLE 6
Sequence of IgG4-end guide RNAs, targeting the
3′end of the CH3 exon in IgG4

| | | | | Off- |
|---|---|---|---|---|
| | | | | target |
| | | | indel | (IGG) |
| | Sequence | SEQ | effi- | indels |
| Guide | targeted by gRNA | ID | ciency, | detected |
| Identity | (5′-3′) | NO: | ICE (%) | by ICE |
| IgG4end- | TCTCTGGGTAAATGAGTGCC | 37 | 8.5 | — |
| g1 | | | | |
| IgG4end- | AAGAGCCTCTCCCTGTCTCT | 38 | 12.5 | — |
| g2 | | | | |
| IgG4end- | CGTGGACAAGAGCAGGTGGC | 39 | 16.3 | — |
| g3 | | | | |
| IgG4end- | GACAAGAGCAGGTGGCAGGA | 40 | 32.3 | — |
| g4 | | | | |
| IgG4end- | GGACAAGAGCAGGTGGCAGG | 41 | 24.0 | — |
| g5 | | | | |

Editing to create full length (H plus L chain) antibodies, including CrossMab designs.

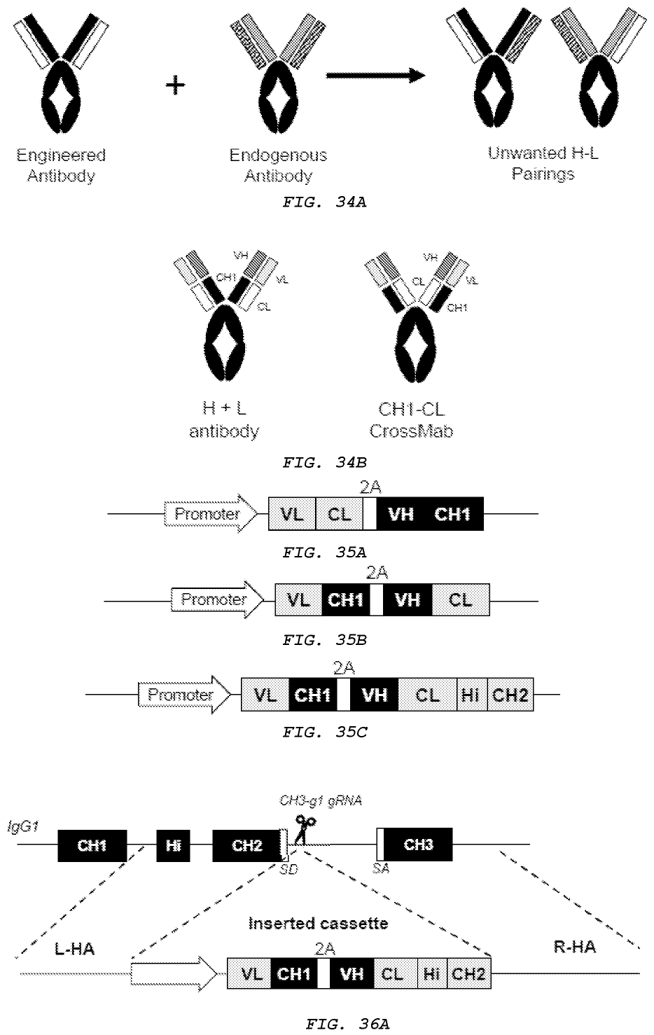

The H chain editing strategies described herein support the insertion of antigen-binding domains derived from cassettes comprising a complete Light chain plus a partial Heavy chain. In a specific modification, the constructs use a CrossMab design, wherein the positions of the CL and CH1 domains are reversed and are present on the alternate antibody chains (FIG. 34). Such designs preserve formation of the introduced and desired antibody cassette while preventing pairing of the introduced antibody chains with endogenous H or L chains expressed in a B cell, thereby reducing the potential for formation of heterologous antibody chain combinations with unwanted specificities, for example self-reactive antibodies.

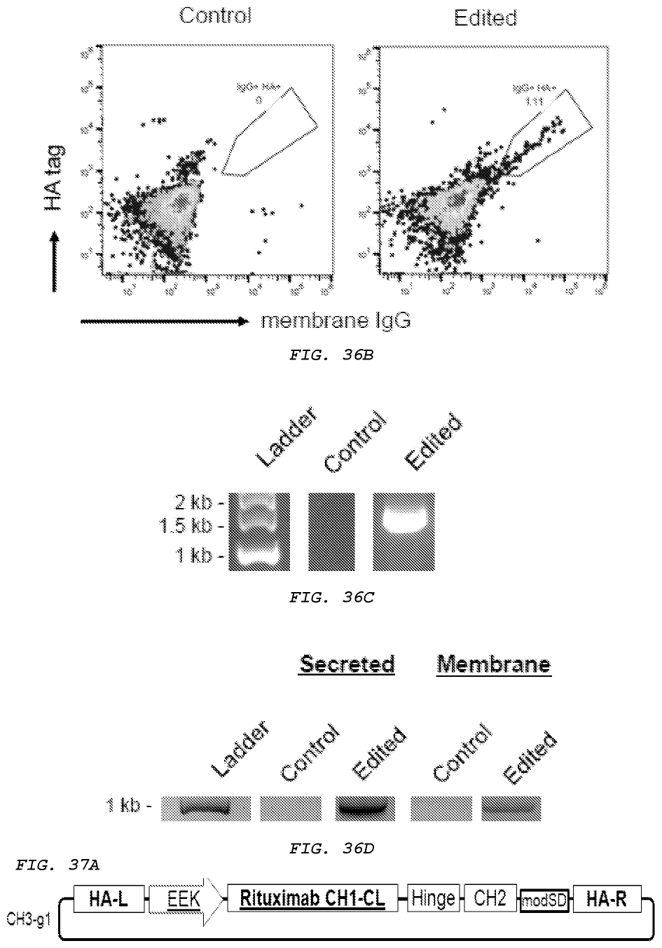

FIG. 35 shows examples of various cassettes that can be introduced into IgH, containing different extant of Heavy chain components, as would be selected based on the insertion site in IgH to be used.

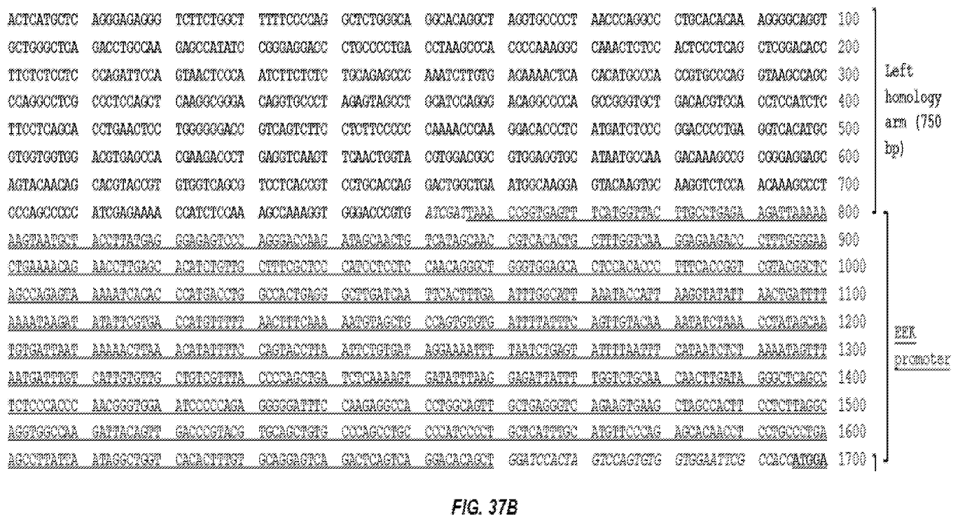

FIG. 36 shows an example of such an approach, editing the IgG1 locus to express an H plus L chain CrossMab antibody with an antigen-binding domain derived from the monoclonal antibody, Rituximab. In this example, the inserted cassette comprises a promoter (EEK) followed by the antibody domains VL-CL-2A-VH-CH1 (FIG. 36A). Following site-specific gene editing using the CH3 g1 gRNA, as H+L Crossmab antibody was expressed (FIG. 36B-D).

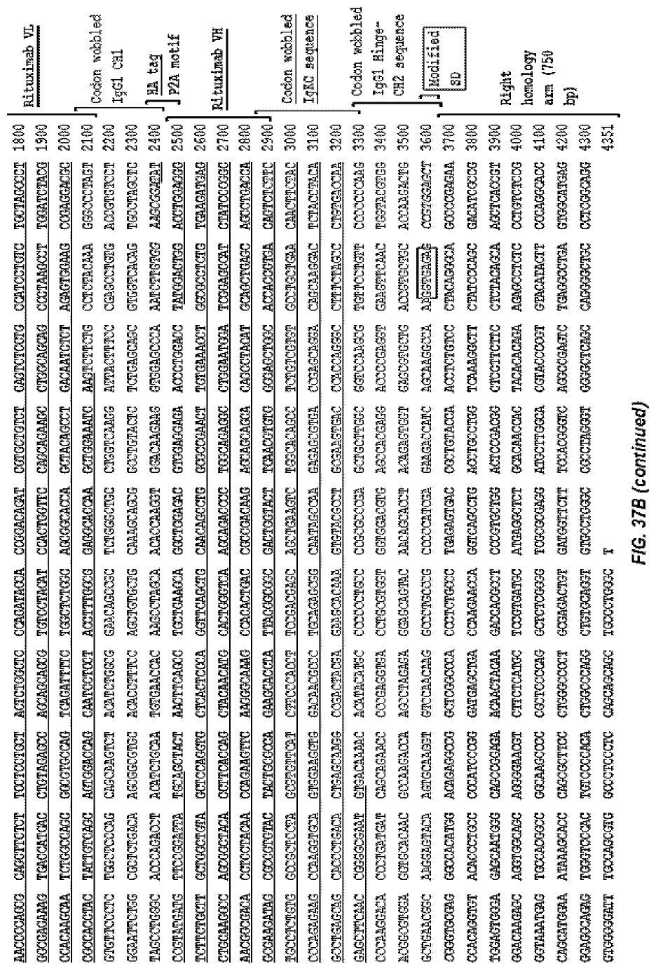

FIG. 37 shows the specific sequence of the homology donor used to generate the example of the Rituximab CrossMab (SEQ ID NO:42).

It will be understood that various modifications may be made without departing from the spirit and scope of this disclosure. Accordingly, other embodiments are within the scope of the following claims.

What is claimed is:

1. A method for the production of antibody fragments or non-immunoglobulin binding domains from an immunoglobulin locus, comprising:
introducing a targeted DNA break in an immunoglobulin locus using a genome editing system; and
inserting a promoter-driven expression construct, that expresses an antigen-binding domain, into the genome edited immunoglobulin locus, wherein the promoter-driven expression construct produces an mRNA encoding an antibody fragment or non-immunoglobulin binding domain.

2. The method of claim 1, wherein the immunoglobulin locus is a human immunoglobulin locus.

3. The method of claim 1, wherein the immunoglobulin locus is selected from the IGHG1, IGHG2, IGHG3, IGHG4, IGHD, IGHE, IGHM, IGHA1, and IGHA2.

4. The method of claim 3, wherein the immunoglobulin locus is selected from the IGHG1, IGHG2, IGHG3, and IGHG4.

5. The method of claim 4, wherein the immunoglobulin locus is IGHG1.

6. The method of claim 1, wherein the genome editing system is selected from CRISPR/Cas9, CRISPR/Cpf1, Zinc finger nucleases (ZFN), and transcription activator-like effector nucleases (TALEN).

7. The method of claim 6, wherein the genome editing system is a CRISPR/Cas9 genome editing system.

8. The method of claim 7, wherein the spCas9 guide RNAs target a polynucleotide having the sequence of sg01, sg02, sg03, sg04, sg05, sg06, sg12, sg16, or sg17 presented in Table 2.

9. The method of claim 6, wherein the genome editing system is a CRISPR/Cpf1 genome editing system.

10. The method of claim 9, wherein the Cpf1 guide RNAs target a polynucleotide having the sequence of Cpf1-g1, Cpf1-g2, Cpf1-g3, or Cpf1-g4 presented in Table 3.

11. The method of claim 6, wherein the genome editing system comprises a guide RNA (gRNA) that targets a sequence as set forth in Table 2, 3, 4 or 6.

12. The method of claim 1, wherein the targeted DNA break (i) is in a constant region downstream of a CH1 exon, (ii) is between the CH1 exon and Hinge exon, (ii) is in an intron between CH2 and CH3 region, and/or (iv) is downstream of a CH2 exon, of the immunoglobulin locus.

13. The method of claim 1, wherein the promoter-driven expression construct is inserted into the genome edited immunoglobulin locus by homology-directed repair.

14. The method of claim 1, wherein the promoter-driven expression construct comprises a B cell specific promoter.

15. The method of claim 14, wherein the B cell specific promoter is an EEK promoter or an MH promoter.

16. The method of claim 1, wherein the promoter-driven expression construct produces an mRNA that further comprises an M1 and an M2 exons of an immunoglobulin locus.

17. A method to produce an engineered B cell or an engineered precursor B cell that expresses an antibody fragment or non-immunoglobulin binding domain, comprising:
treating a B cell or a precursor B cell using the method of claim 1.

18. The method of claim 17, wherein the B cell or the precursor B cell is engineered ex vivo, in vitro or in vivo.

19. An engineered B cell or an engineered precursor B cell that expresses an antibody fragment or non-immunoglobulin binding domain made by the method of claim 17.

20. A cell line comprising the engineered B cell or an engineered precursor B cell of claim 19.

21. The cell line of claim 20, wherein the engineered precursor B cell comprises an embryonic stem cell, a hematopoietic stem cell or an induced pluripotent stem cell.

22. An antibody fragment or non-immunoglobulin binding domain isolated from the engineered B cell or an engineered precursor B cell of claim 19.

23. A method of treating a subject with a microbial or viral infection, comprising:
obtaining isolated B cells or precursor B cells;
treating the isolated B cells or precursor B cells with the method of claim 1 to produce engineered B cells or engineered precursor B cells that express an antibody fragment or non-immunoglobulin binding domain that recognize antigen(s) from the infectious microbe or virus;
administering the engineered B cells or engineered precursor B cells to the subject.

24. The method of claim 23, wherein the isolated B cells or precursor B cells are autologous to the subject.

25. The method of claim 23, wherein the isolated B cells or precursor cells are allogeneic to the subject.

26. The method of claim 23, wherein the viral infection is HIV, Hepatitis, Herpes simplex, Ebola, Dengue, influenza, and coronavirus.

27. A method of treating a subject with cancer, comprising:
obtaining isolated B cells or precursor B cells;
treating the isolated B cells or precursor B cells with the method of claim 1 to produce engineered B cells or engineered precursor B cells that expresses antibody fragments or non-immunoglobulin binding domains that recognize antigen(s) from a cancer cell;
administering the engineered B cells or engineered precursor B cells to the subject.

28. The method of claim 27, wherein the isolated B cells or precursor B cells are autologous to the subject.

29. The method of claim 27, wherein the isolated B cells or precursor cells are allogeneic to the subject.

30. The method of claim 27, wherein the subject has a cancer selected from non-Hodgkin's lymphoma, acute lymphoblastic leukemia, B-cell lymphoma, mantle cell lymphoma, multiple myeloma, acute myeloid leukemia, colorectal cancer, breast cancer, lung cancer, ovarian cancer, and renal cancer.

31. A method of treating a subject with an autoimmune disorder, comprising:
obtaining isolated B cells or precursor B cells;
treating the isolated B cells or precursor B cells with the method of claim 1 to produce engineered B cells or engineered precursor B cells that expresses antibody fragments or non-immunoglobulin binding domains that can bind to and prevent activation of cytokines or receptors associated with an autoimmune disorder, or prevent aggregations or plaques associated with an autoimmune disorder;
administering the engineered B cells or engineered precursor B cells to the subject.

32. The method of claim 31, wherein the isolated B cells or precursor B cells are autologous to the subject.

33. The method of claim 31, wherein the isolated B cells or precursor cells are allogeneic to the subject.

34. The method of claim 31, wherein the subject has an autoimmune disorder selected from Alzheimer's disease, Celiac disease, Addison disease, Graves disease, dermatomyositis, multiple sclerosis, rheumatoid arthritis, psoriasis, and inflammatory bowel disease.

35. A polynucleotide comprising:
an antigen recognition cassette comprising a promoter operably linked to a sequence encoding a binding domain and a splice donor site compatible with an immunoglobulin exon sequence splice acceptor.

36. The polynucleotide of claim 35, further comprising at least one homology arm at the 5′ and/or 3′ end of the antigen recognition cassette.

37. The polynucleotide of claim 35, wherein the polynucleotide is present in a vector.

38. The polynucleotide of claim 37, wherein the vector is a viral vector.

39. The polynucleotide of claim 38, wherein the vector is an adeno-associated virus (AAV).

40. The polynucleotide of claim 35, wherein the promoter is a promoter functional in a mammalian cell.

41. The polynucleotide of claim 40, wherein the mammalian cell is a mammalian B-cell or B-cell precursor.

42. The polynucleotide of claim 41, wherein the B-cell precursor is an induced pluripotent stem cell, a hematopoietic stem cell or an embryonic stem cell.

43. The polynucleotide of claim 35, wherein the promoter is a constitutive promoter.

44. The polynucleotide of claim 35, wherein the promoter is an inducible promoter.

45. The polynucleotide of claim 35, wherein the binding domain comprises an antibody fragment.

46. The polynucleotide of claim 35, wherein the binding domain is a non-immunoglobulin polypeptide binding domain.

47. The polynucleotide of claim 35, wherein the binding domain interacts with an antigen selected from the group consisting of glycoproteins; bacterial or viral antigens; CD3, CD5; CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors; a glycosylated CD43 epitope expressed on non-hematopoietic cancers; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor); carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ES0-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites); Squamous Cell Carcinoma Antigen Recognized By T Cells 3 (SART3); Paired box protein Pax-5 (PAX5); proacrosin binding protein sp32 (OY-TESl); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl); MPL; c-MYC epitope Tag; CD34; LAMP1; TROP2; GFRalpha4; CDH17; CDH6; NYBR1; CDH19; CD200R; Slea (CA19.9); Sialyl Lewis Antigen); Fucosyl-GM1; PTK7; gpNMB; CDH1-CD324; DLL3; CD276/B7H3; IL11Ra; IL13Ra2; CD179b-IGLl1; TCRgamma-delta; NKG2D; CD32 (FCGR2A); CD16 (FGCR3A), Tn ag; Timl-/HVCR1; CSF2RA (GM-CSFR-alpha); TGFbetaR2; Lews Ag; TCR-beta1 chain; TCR-beta2 chain; TCR-gamma chain; TCR-delta chain; FITC; Leutenizing hormone receptor (LHR); Follicle stimulating hormone receptor (FSHR); Gonadotropin Hormone receptor (CGHR or GR); CCR4; GD3; SLAMF6; SLAMF4; HIV1 envelope glycoprotein; HTLV1-Tax; CMV pp65; EBV-EBNA3c; KSHV K8.1; KSHV-gH; influenza A hemagglutinin (HA); GAD; PDL1; Guanylyl cyclase C (GCC); auto antibody to desmoglein 3 (Dsg3); auto antibody to desmoglein 1 (Dsg1); HLA; HLA-A; HLA-A2; HLA-B; HLA-C; HLA-DP; HLA-DM; HLA-DOA; HLA-DOB; HLA-DQ; HLA-DR; HLA-G; IgE; CD99; Ras G12V; Tissue Factor 1 (TF1); AFP; GPRC5D; Claudin18.2 (CLD18A2 or CLDN18A.2); P-glycoprotein; STEAP1; Liv1; Nectin-4; Cripto; gpA33; BST1/CD157; low conductance chloride channel; and the antigen recognized by TNT antibody.

48. The polynucleotide of claim 35, wherein the immunoglobulin exon sequence splice acceptor is downstream of the CH1 exon.

49. A recombinant B cell or B cell precursor comprising a heterologous promoter linked to a binding domain coding sequence and a splice donor engineered into an immunoglobulin locus of the B cell or B cell precursor.

50. The recombinant B cell or B cell precursor of claim 49, wherein the heterologous promoter is a promoter functional in a mammalian cell.

51. The recombinant B cell or B cell precursor of claim 49, wherein the B-cell precursor is an induced pluripotent stem cell, a hematopoietic stem cell or an embryonic stem cell.

52. The recombinant B cell or B cell precursor of claim 49, wherein the promoter is a constitutive promoter.

53. The recombinant B cell or B cell precursor of claim 49, wherein the promoter is an inducible promoter.

54. The recombinant B cell or B cell precursor of claim 49, wherein the binding domain coding sequence encodes an antibody fragment.

55. The recombinant B cell or B cell precursor of claim 49, wherein the binding domain coding sequence encodes a non-immunoglobulin polypeptide binding domain.

56. The recombinant B cell or B cell precursor of claim 49, wherein the binding domain interacts with an antigen selected from the group consisting of glycoproteins; bacterial or viral antigens; CD3, CD5; CD19; CD123; CD22; CD30; CD171; CS1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRviii); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms Like Tyrosine Kinase 3 (FLT3); Tumor-associated glycoprotein 72 (TAG72); CD38; CD44v6; a glycosylated CD43 epitope expressed on acute leukemia or lymphoma but not on hematopoietic progenitors; a glycosylated CD43 epitope expressed on non-hematopoietic cancers; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha (FRa or FR1); Folate receptor beta (FRb); Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor); carbonic anhydrase IX (CAlX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); sialyl Lewis adhesion molecule (sLe); ganglioside GM3 (aNeu5Ac(2-3)bDClalp(1-4)bDGlcp(1-1)Cer); transglutaminase 5 (TGS5); high molecular weight-melanoma associated antigen (HMWMAA); o-acetyl-GD2 ganglioside (OAcGD2); tumor endothelial marker 1 (TEM1/CD248); tumor endothelial marker 7-related (TEM7R); claudin 6 (CLDN6); thyroid stimulating hormone receptor (TSHR); G protein coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ES0-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGEl); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCT A-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites); Squamous Cell Carcinoma Antigen Recognized By T Cells 3 (SART3); Paired box protein Pax-5 (PAX5); proacrosin binding protein sp32 (OY-TESl); lymphocyte-specific protein tyrosine kinase (LCK); A kinase anchor protein 4 (AKAP-4); synovial sarcoma, X breakpoint 2 (SSX2); Receptor for Advanced Glycation End products (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRl); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLLl); MPL; c-MYC epitope Tag; CD34; LAMP1; TROP2; GFRalpha4; CDH17; CDH6; NYBR1; CDH19; CD200R; Slea (CA19.9); Sialyl Lewis Antigen); Fucosyl-GM1; PTK7; gpNMB; CDH1-CD324; DLL3; CD276/B7H3; IL11Ra; IL13Ra2; CD179b-IGLl1; TCRgamma-delta; NKG2D; CD32 (FCGR2A); CD16 (FGCR3A), Tn ag; Timl-/HVCR1; CSF2RA (GM-CSFR-alpha); TGFbetaR2; Lews Ag; TCR-beta1 chain; TCR-beta2 chain; TCR-gamma chain; TCR-delta chain; FITC; Leutenizing hormone receptor (LHR); Follicle stimulating hormone receptor (FSHR); Gonadotropin Hormone receptor (CGHR or GR); CCR4; GD3; SLAMF6; SLAMF4; HIV1 envelope glycoprotein; HTLV1-Tax; CMV pp65; EBV-EBNA3c; KSHV K8.1; KSHV-gH; influenza A hemagglutinin (HA); GAD; PDL1; Guanylyl cyclase C (GCC); auto antibody to desmoglein 3 (Dsg3); auto antibody to desmoglein 1 (Dsg1); HLA; HLA-A; HLA-A2; HLA-B; HLA-C; HLA-DP; HLA-DM; HLA-DOA; HLA-DOB; HLA-DQ; HLA-DR; HLA-G; IgE; CD99; Ras G12V; Tissue Factor 1 (TF1); AFP; GPRC5D; Claudin18.2 (CLD18A2 or CLDN18A.2); P-glycoprotein; STEAP1; Liv1; Nectin-4; Cripto; gpA33; BST1/CD157; low conductance chloride channel; and the antigen recognized by TNT antibody.


SEQUENCE LISTING (42 sequences)

SEQ ID NO: 1 - DNA, 23 bp, synthetic construct
  source 1..23: mol_type = other DNA
tccgggtgaagaggcagacgngg

SEQ ID NO: 2 - DNA, 17 bp, synthetic construct
  source 1..17: mol_type = other DNA
tccgggtgaagaggcag

SEQ ID NO: 3 - DNA, 68 bp, synthetic construct
  source 1..68: mol_type = other DNA
ttctggctttttccccaggctctgggcaggcacaggctaggtgcccctaaatcgatggggttggggtt

SEQ ID NO: 4 - DNA, 68 bp, synthetic construct
  source 1..68: mol_type = other DNA
cgcatcccggctatgcagccccagtccagggcagcaaggcaggccccgtcatcgatggggttggggtt

SEQ ID NO: 5 - DNA, 68 bp, synthetic construct
  source 1..68: mol_type = other DNA
acgcatcccggctatgcagccccagtccagggcagcaaggcaggccccgtatcgatggggttggggtt

SEQ ID NO: 6 - DNA, 68 bp, synthetic construct
  source 1..68: mol_type = other DNA
aggccaaactctccactccctcagctcggacaccttctctcctcccagatatcgatggggttggggtt

SEQ ID NO: 7 - DNA, 68 bp, synthetic construct
  source 1..68: mol_type = other DNA
ggctatgcagccccagtccagggcagcaaggcaggccccgtctgcctcttatcgatggggttggggtt

SEQ ID NO: 8 - DNA, 75 bp, synthetic construct
  source 1..75: mol_type = other DNA
gcagccccagtccagggcagcaaggcaggccccgtatcgattaaaccggtgagtttcatggttacttgcctgaga

SEQ ID NO: 9 - DNA, 65 bp, synthetic construct
  source 1..65: mol_type = other DNA
gcagccccagtccagggcagcaaggcaggccccgtatcgatggggttggggttgcgccttttcca

SEQ ID NO: 10 - DNA, 51 bp, synthetic construct
  source 1..51: mol_type = other DNA
gccaggagcaagavcacctacatcavctayaacavcaacgvctacgactac

SEQ ID NO: 11 - protein, 16 aa, synthetic construct
ARXKXTYIXYNXNXYD

SEQ ID NO: 12 - DNA, 1364 bp, synthetic construct
  source 1..1364: mol_type = other DNA
taaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctggatccactagtccagtgtggtggaattcaccatggagtttgggctgagctggctttttcttgtggctattttaaaaggtgtccagtgtgaggtgcagctggtggagtctgggggaggcttggtacaggccggggggttcctgagactctcctgtgagctgaggggaagcatctttaaccagtatgccatggcctggttccgccaggctccagggaaggagagggagttcgtcgccggcatgggcgccgtgccccactacggcgagttcgtgaagggccggttcaccatctccagagacaatgccaagagcacggtgtatctgcaaatgagcagcctgaagcccgaggacacggccatctatttctgtgccaggagcaagagcacctacatcagctacaacagcaacggctacgactactggggcaggggaacccaggtcaccgtctcctcaggtgagag

SEQ ID NO: 13 - DNA, 2373 bp, synthetic construct
  source 1..2373: mol_type = other DNA
gaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtatcgattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctggatccactagtccagtgtggtggaattcaccatggagtttgggctgagctggctttttcttgtggctattttaaaaggtgtccagtgtgaggtgcagctggtggagtctgggggaggcttggtacaggccggggggttcctgagactctcctgtgagctgaggggaagcatctttaaccagtatgccatggcctggttccgccaggctccagggaaggagagggagttcgtcgccggcatgggcgccgtgccccactacggcgagttcgtgaagggccggttcaccatctccagagacaatgccaagagcacggtgtatctgcaaatgagcagcctgaagcccgaggacacggccatctatttctgtgccaggagcaagagcacctacatcagctacaacagcaacggctacgactactggggcaggggaacccaggtcaccgtctcctcaggtgagagctcctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacacc

SEQ ID NO: 14 - DNA, 3207 bp, synthetic construct
  source 1..3207: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctgaggccaagctagagacactggactgtgctgactcccggcaggcacagagcgctgacctggctgccgagccccgcctcctaggctgcaggggtgcctgcagaagggcaccacagggccaccggtcctgcaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtatcgattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctggatccactagtccagtgtggtggaattcaccatggagtttgggctgagctggctttttcttgtggctattttaaaaggtgtccagtgtgaggtgcagctggtggagtctgggggaggcttggtacaggccggggggttcctgagactctcctgtgagctgaggggaagcatctttaaccagtatgccatggcctggttccgccaggctccagggaaggagagggagttcgtcgccggcatgggcgccgtgccccactacggcgagttcgtgaagggccggttcaccatctccagagacaatgccaagagcacggtgtatctgcaaatgagcagcctgaagcccgaggacacggccatctatttctgtgccaggagcaagagcacctacatcagctacaacagcaacggctacgactactggggcaggggaacccaggtcaccgtctcctcaggtgagagctcctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagagtcgacctgcagaagcttgcctcgagcagcgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 15 - DNA, 20 bp, synthetic construct
  source 1..20: mol_type = other DNA
aggctaggtgcccctaaccc

SEQ ID NO: 16 - DNA, 20 bp, synthetic construct
  source 1..20: mol_type = other DNA
tagccgggatgcgtccaggc

SEQ ID NO: 17 - DNA, 20 bp, synthetic construct
  source 1..20: mol_type = other DNA
tgcatagccgggatgcgtcc

SEQ ID NO: 18 - DNA, 20 bp, synthetic construct
  source 1..20: mol_type = other DNA
ctccgggtgaagaggcagac

SEQ ID NO: 19 - DNA, 20 bp, synthetic construct
  source 1..20: mol_type = other DNA
tccgggtgaagaggcagacg

SEQ ID NO: 20 - DNA, 20 bp, synthetic construct
  source 1..20: mol_type = other DNA
acccaggccctgcacacaaa

SEQ ID NO: 21 - DNA, 20 bp, synthetic construct
  source 1..20: mol_type = other DNA
gattgggagttactggaatc

SEQ ID NO: 22 - DNA, 20 bp, synthetic construct
  source 1..20: mol_type = other DNA
gcagaggcctccgggtgaag

SEQ ID NO: 23 - DNA, 20 bp, synthetic construct
  source 1..20: mol_type = other DNA
gccccgtctgcctcttcacc

SEQ ID NO: 24 - DNA, 20 bp, synthetic construct
  source 1..20: mol_type = other DNA
ccgtctgcctcttcacccgg

SEQ ID NO: 25 - DNA, 21 bp, synthetic construct
  source 1..21: mol_type = other DNA
tccccaggctctgggcaggca

SEQ ID NO: 26 - DNA, 21 bp, synthetic construct
  source 1..21: mol_type = other DNA
ccccaggctctgggcaggcac

SEQ ID NO: 27 - DNA, 21 bp, synthetic construct
  source 1..21: mol_type = other DNA
cccaggctctgggcaggcaca

SEQ ID NO: 28 - DNA, 21 bp, synthetic construct
  source 1..21: mol_type = other DNA
tgtgcagggcctgggttaggg

SEQ ID NO: 29 - DNA, 20 bp, synthetic construct
  source 1..20: mol_type = other DNA
atgtggccctcgcaccccac

SEQ ID NO: 30 - DNA, 20 bp, synthetic construct
  source 1..20: mol_type = other DNA
aagccaaaggtgggacccgt

SEQ ID NO: 31 - DNA, 20 bp, synthetic construct
  source 1..20: mol_type = other DNA
agccaaaggtgggacccgtg

SEQ ID NO: 32 - DNA, 20 bp, synthetic construct
  source 1..20: mol_type = other DNA
gtgggacccgtggggtgcga

SEQ ID NO: 33 - DNA, 20 bp, synthetic construct
  source 1..20: mol_type = other DNA
catgtggccctcgcacccca

SEQ ID NO: 34 - DNA, 3256 bp, synthetic construct
  source 1..3256: mol_type = other DNA
actcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtgatcgattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctggatccactagtccagtgtggtggaattcaccatggagtttgggctgagctggctttttcttgtggctattttaaaaggtgtccagtgtgaggtgcagctggtggagtctgggggaggcttggtacaggccggggggttcctgagactctcctgtgagctgaggggaagcatctttaaccagtatgccatggcctggttccgccaggctccagggaaggagagggagttcgtcgccggcatgggcgccgtgccccactacggcgagttcgtgaagggccggttcaccatctccagagacaatgccaagagcacggtgtatctgcaaatgagcagcctgaagcccgaggacacggccatctatttctgtgccaggagcaagagcacctacatcagctacaacagcaacggctacgactactggggcaggggaacccaggtcaccgtgagcagcgagccgaagagctgtgacaaaacacatacatgccccccctgccccgcgcccgagctgcttggcggtccaagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgtccaacaaggccctgcccgcccccatcgagaagaccatcagcaaggccaaaggtgagagccgtggagctcgggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtgcaggtgtgcctgggccgcctagggtggggctcagccaggggctgccctcggcagggtgggggatttgccagcgtggccctccctccagcagcagctgccctgggct

SEQ ID NO: 35 - DNA, 3256 bp, synthetic construct
  source 1..3256: mol_type = other DNA
actcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtgatcgattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctggatccactagtccagtgtggtggaattcaccatggagtttgggctgagctggctttttcttgtggctattttaaaaggtgtccagtgtgaggtgcagctggtggagtctgggggaggcttggtacaggccggggggttcctgagactctcctgtgagctgaggggaagcatctttaaccagtatgccatggcctggttccgccaggctccagggaaggagagggagttcgtcgccggcatgggcgccgtgccccactacggcgagttcgtgaagggccggttcaccatctccagagacaatgccaagagcacggtgtatctgcaaatgagcagcctgaagcccgaggacacggccatctatttctgtgccaggagcaagagcacctacatcagctacaacagcaacggctacgactactggggcaggggaacccaggtcaccgtgagcagcgagccgaagagctgtgacaaaacacatacatgccccccctgccccgcgcccgagctgcttgccggtccagacgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgtccaacaaggccctgcccctgcccgaggagaagaccatcagcaaggccaaaggtgagagccgtggagctcgggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtgcaggtgtgcctgggccgcctagggtggggctcagccaggggctgccctcggcagggtgggggatttgccagcgtggccctccctccagcagcagctgccctgggct

SEQ ID NO: 36 - protein, 8 aa, synthetic construct
GASDALIE

SEQ ID NO: 37 - DNA, 20 bp, synthetic construct
  source 1..20: mol_type = other DNA
tctctgggtaaatgagtgcc

SEQ ID NO: 38 - DNA, 20 bp, synthetic construct
  source 1..20: mol_type = other DNA
aagagcctctccctgtctct

SEQ ID NO: 39 - DNA, 20 bp, synthetic construct
  source 1..20: mol_type = other DNA
cgtggacaagagcaggtggc

SEQ ID NO: 40 - DNA, 20 bp, synthetic construct
  source 1..20: mol_type = other DNA
gacaagagcaggtggcagga

SEQ ID NO: 41 - DNA, 20 bp, synthetic construct
  source 1..20: mol_type = other DNA
ggacaagagcaggtggcagg

SEQ ID NO: 42 - DNA, 4351 bp, synthetic construct
  source 1..4351: mol_type = other DNA
actcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtgatcgattaaaccggtgagtttcatggttacttgcctgagaagattaaaaaaagtaatgctaccttatgagggagagtcccagggaccaagatagcaactgtcatagcaaccgtcacactgctttggtcaaggagaagaccctttggggaactgaaaacagaaccttgagcacatctgttgctttcgctcccatcctcctccaacagggctgggtggagcactccacaccctttcaccggtcgtacggctcagccagagtaaaaatcacacccatgacctggccactgagggcttgatcaattcactttgaatttggcattaaataccattaaggtatattaactgattttaaaataagatatattcgtgaccatgtttttaactttcaaaaatgtagctgccagtgtgtgattttatttcagttgtacaaaatatctaaacctatagcaatgtgattaataaaaacttaaacatattttccagtaccttaattctgtgataggaaaattttaatctgagtattttaatttcataatctctaaaatagtttaatgatttgtcattgtgttgctgtcgtttaccccagctgatctcaaaagtgatatttaaggagattattttggtctgcaacaacttgatagggctcagcctctcccacccaacgggtggaatcccccagagggggatttccaagaggccacctggcagttgctgagggtcagaagtgaagctagccacttcctcttaggcaggtggccaagattacagttgacccgtacgtgcagctgtgcccagcctgccccatcccctgctcatttgcatgttcccagagcacaacctcctgccctgaagccttattaataggctggtcacactttgtgcaggagtcagactcagtcaggacacagctggatccactagtccagtgtggtggaattcgccaccatggaaaccccagcgcagcttctcttcctcctgctactctggctcccagataccaccggacagatcgtgctgtctcagtctcctgccatcctgtctgctagccctggcgagaaagtgaccatgacctgtagagccagcagcagcgtgtcctacatccactggttccagcagaagcctggcagcagccctaagccttggatctacgccacaagcaatctggccagcggcgtgccagtcagattttctggctctggcagcggcaccagctacagcctgacaatctctagagtggaagccgaggacgccgccacctactattgtcagcagtggaccagcaatcctcctacctttggcggaggcaccaagctggaaatcaagtcttctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggagcccaaatcttgtggaagcggatatccgtatgatgttccggattatgcagctactaacttcagcctgctgaagcaggctggagacgtggaggagaaccctggacctatggactggacctggagggtcttctgcttgctggctgtagctccaggtgctcactcccaggttcagctgcaacagcctggcgccgaacttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtctcttctgcctctgtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgtgacaaaacacatacatgccccccctgccccgcgcccgagctgcttggcggtccaagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcctagagaggagcagtacaacagcacctacagagtggtgagcgtgctgaccgtgctgcaccaagactggctgaacggcaaggagtacaagtgcaaggtgtccaacaaggccctgcccgcccccatcgagaagaccatcagcaaggccaaaggtgagagccgtggagctcgggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtgcaggtgtgcctgggccgcctagggtggggctcagccaggggctgccctcggcagggtgggggatttgccagcgtggccctccctccagcagcagctgccctgggct